(19) United States
(12) Patent Application Publication
(10) Pub. No.: US 2017/0151416 A1
(43) Pub. Date: Jun. 1, 2017

(54) Methods and Systems for Delivery of a Trail of a Therapeutic Substance into an Anatomical Space

(71) Applicant: InVivo Therapeutics Corporation, Cambridge, MA (US)

(72) Inventors: Artem B. Kutikov, Somerville, MA (US); Richard T. Layer, Marlboro, MA (US); Simon W. Moore, Waltham, MA (US); Thomas R. Ulich, New York, NY (US); James D. Guest, Miami, FL (US); Alex A. Aimetti, Sudbury, MA (US); Robert Charles, New Boston, NH (US); Brendan P. Collins, Manchester, NH (US); Jon Taylor, Groton, MA (US); Eugene Zeleny, Newton, MA (US)

(21) Appl. No.: 15/361,985

(22) Filed: Nov. 28, 2016

Related U.S. Application Data
(60) Provisional application No. 62/384,505, filed on Sep. 7, 2016.
(60) Provisional application No. 62/261,622, filed on Dec. 1, 2015.

(51) Int. Cl. A61M 25/00; A61B 90/11; A61M 25/06; A61K 35/30; A61L 29/06; A61L 29/14; A61L 29/02; A61M 5/00; A61M 5/142

(52) U.S. Cl. CPC A61M 25/0084; A61M 5/007; A61B 90/11; A61M 25/0662; A61M 5/14216; A61L 29/06; A61L 29/14; A61L 29/02; A61K 35/30; A61M 2025/0007; A61M 2025/0089; A61M 2025/0175

(57) ABSTRACT

Injection devices and methods for delivering a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, to treat an injury or disorder of the central nervous system requiring injection of cells and/or one more therapeutic substances. The devices and methods are useful for the treatment of a variety of traumas, conditions and diseases, in particular, spinal cord injuries, amyotrophic lateral sclerosis, multiple sclerosis and spinal ischemia as well as other spinal cord degenerative conditions and pathologies.

### CROSS-REFERENCE TO RELATED APPLICATIONS

This application claims the benefit of U.S. Provisional Application No. 62/261,622, filed on Dec. 1, 2015 and U.S. Provisional Application No. 62/384505, filed on Sep. 7, 2016, and also claims priority to non-provisional patent application Ser. No. 15/362,257 entitled COMPOSITIONS AND METHODS FOR PREPARING AN INJECTABLE MEDIUM FOR ADMINISTRATION INTO THE CENTRAL NERVOUS SYSTEM filed on the same date as the present application. The entire disclosure of each of the aforesaid applications is incorporated by reference in the present application.

### FIELD OF INVENTION

The present invention is generally directed to an injection system and associated methods for administration of medical treatments to traumatized or diseased organs and/or tissues by transplantation and/or delivery of cells and/or at least one therapeutic substance, or diagnostic agent or other injectable medium directly into a desired anatomical space of an animal or human subject; for example, by injection directly into the spinal cord parenchyma, by injecting a trail of therapeutic cells and/or at least one therapeutic substance, diagnostic substance, or other injectable medium in the traumatized and/or diseased area of the respective anatomical space. Conversely, the described system and methods may be employed to remove fluids from traumatized or diseased anatomical spaces of organs or tissues of an animal or human subject.

### BACKGROUND OF THE INVENTION

Specifically, an apparatus and method is provided for safely accessing anatomical spaces with surfaces to deliver medical devices or media into such spaces, or to remove fluids from such spaces.

In the surgical setting, a surgeon is frequently confronted with the need to safely access an anatomical space of an organ or other tissue for the purpose of delivering or administering therapeutic cells, and/or at least one therapeutic substance, or diagnostic substance or other injectable medium to treat a trauma to such organ or other tissue, or to treat a disease or other medical condition. Conversely, the surgeon or other medical practitioner may desire to remove a fluid from such an anatomical space of the body of an animal or human subject. A particular need exists to administer therapeutic cells, and/or at least one therapeutic substance, or diagnostic substance or other injectable medium to the central nervous system, in particular, to areas of the brain and the spinal cord.

Spinal cord injuries may result in paraplegia or quadriplegia in a substantial number of subjects. Over 270,000 people live with chronic spinal cord injury in the U.S. alone with approximately 12,000 traumatic spinal cord injury occurring per year. The delivery of therapeutic substances, such as therapeutic cells and/or therapeutic drug substances, such as growth factors, antibodies, analgesics, anesthetics and the like, or diagnostic substances or other injectable medium into the spinal cord, may be useful in the treatment of spinal cord injuries, and a number of medical diseases and conditions, including amyotrophic lateral sclerosis (“ALS”), multiple sclerosis (“MS”), spinal muscular atrophy (“SMA”) and spinal ischemia as well as other spinal cord degenerative conditions and pathologies.

Prior delivery strategies for the injection of therapeutic cells and/or at least one therapeutic substance, or diagnostic substance or other injectable medium into the central nervous system have a number of limitations. Some injection strategies require multiple injection sites, thereby resulting in concentrated and localized delivery sites for cells and/or other therapeutic substances. For instance, in one procedure multiple vertical spinal cord injections are required to deliver cells into multiple spinal cord segments. Such a procedure presents risks to the patient, such as infection and loss of cerebrospinal fluid and the attendant sequelae due to the multiple injections required.

Injections of the type described may, for example, cause injury at each site of injection; deliver inaccurate doses as a result of cell reflux up the needle track; have limited surface area for cellular integration, or require lengthy procedure times. . Furthermore, in the case of cell therapy for spinal cord injury where the creation of a functional neuronal relay across or around an injury is desired, discrete bolus injections of cells may not form sufficient connections across the bolus to bolus injection distance. In contrast, a continuous trail of cells may from a relay that serves as a novel neuronal column with inputs and outputs at different spatial points to create new connections from the brain/brain stem to the spinal cord.

Delivery of therapeutic cells and/or at least one therapeutic substance, or diagnostic substance or other injectable medium directly into the parenchyma of the spinal cord thus presents numerous challenges to a health care professional. These challenges include the relatively small size of the spinal cord, movement of the spinal cord within multiple planes relative to the surrounding vertebrae, and the known vulnerability of the spinal cord to injury. The same can be said generally with regard to the delivery and administration of therapeutic cells and/or at least one therapeutic substance, or diagnostic substance or other injectable medium to remote anatomical spaces of the body of an animal or human subject. These challenges are further exacerbated when delivering a long interconnected cell relay or trail within the tissue of interest. Therefore, a need exists to provide a surgical technique and associated system for the administration of therapeutic cells and/or at least one therapeutic substance, or diagnostic substance or other injectable medium directly into the traumatized and/or diseased anatomical site of a subject, for instance into the central nervous system, particularly the spinal cord.

Systems known in the art for administration of cells and/or other therapeutic substances into the central nervous system include injections into the brain using multiple injections of cells through flexible, plastic cannulas. The multiple injections result in localized deposition of cells that are not in a single plane. See FIG. 2C of Brecknell and Fawcett, Experimental Neurology, 1996; 138: 338-343. Another administration device employs a rigid guide needle which maintains a specific angle for placement of a flexible injection needle. See page 1498—Material and Methods section of Cunningham et al., Neurosurgery, 2004; 54: 1497-1507.

The procedures and apparatus described in the foregoing references depart significantly from the procedures and apparatus of the present invention, by, for example, utilizing non-motorized flexible cannula as opposed to a motorized injection cannula housed in a guide needle assembly and the lack of control over injection angles that is evident in the prior disclosures. The procedures and apparatus disclosed in the foregoing references would be unsuitable to deliver a long trail of therapeutic cells and/or at least one therapeutic substance, or diagnostic substance or other injectable medium into the narrow diameter (generally on the order of <1 to 1.5 cm) of the spinal cord. The same can also be said to administration and delivery of therapeutic cells and/or at least one therapeutic substance, or diagnostic substance or other injectable medium to remote anatomical spaces of an animal or human subject. The present invention solves these and other administration problems by controlling penetration of the injection needle or flexible injection catheter at a relatively shallow angle, generally on the order of 0-25°. Moreover, it is not feasible to implement the described prior art cranial injection devices and injection procedures for delivery of cells and/or therapeutic substances into the spinal cord because the cranial injection cannulas cannot be deflected through a side hole aperture at such an angle, as disclosed in the references.

Another injection system employs an endoscope comprising a large (10 gauge) needle attached at the distal end and a flexible, steerable endoscope housing a microcatheter, which may be directed intradurally through an introducer sheath. Saline is introduced to distend the subarachnoid space via a syringe in communication with the needle affixed to the endoscope. The microcatheter with attached needle is advanced through a working channel of the endoscope into the dorsal surface of the spinal cord. Cells may then be introduced while the microcatheter is withdrawn slowly to create a trail of cells within the spinal cord. The spinal cord can be visualized through the skin puncture and the endoscope can be navigated under visual guidance. Cells and/or a therapeutic substance can be injected from the needle into the spinal cord. One such system is described in U.S. Pat. No. 7,666,177, the disclosures of which are hereby incorporated by reference in their entirety.

In a particular embodiment, U.S. Pat. No. 7,666,177 (hereinafter, “the '177 patent”) describes a procedure and system that includes injecting a therapeutic substance from a hollow guidewire and withdrawing the guidewire over a period of time to create a trail of therapeutic substance parallel to the longitudinal axis of the spinal cord. Such a system requires use of an endoscope and injection of a fluid to distend the epidural and subarachnoid spaces of the spinal cord, thereby complicating the administration of cells and/or other therapeutic substances.

The '177 patent thus employs an endoscope to access the subarachnoid space of the spinal cord and to introduce a needle to deliver a trail of therapeutic parallel to a longitudinal axis of the cord. Such a procedure is described in Guest, J. et al., Neurosurgery 54(4): 950-955 (3004). Table 1 of the reference publication notes various problems associated with this endoscopic injection approach versus open surgical approaches. These problems include: potential alteration of the subarachnoid space after injury may render the approach unfeasible; visualization through an endoscope is typically poor compared to a surgical microscope; the trajectory of the injection needle may be constrained; and cellular dispersion may be increased in a fluid environment, resulting in seeding outside the desired injection site. These shortcomings are minimized or eliminated through the procedures and use of the apparatus according to the present invention.

The described endoscope-based approach in the '177 patent and the foregoing publication is technically challenging due to the limited spinal cord access and visualization provided by an endoscope. This approach also lacks reproducibility because the trail of therapeutic cannot be stereotaxically positioned within the spinal cord, and the injection procedure and described injection apparatus lack control of trail length and volume due to the described manual approach. The foregoing technical challenges and lack of accuracy may result in the creation of short trails of cells and/or therapeutic substances of only 4-mm in length, as described in the specification. Such a distance is insufficient to bridge most spinal cord lesions. Finally, trails created at an angle with respect to the cord, rather than parallel to the cord axis, may be therapeutically beneficial and these cannot be accomplished with the apparatus and injection procedure specified in the '177 patent.

Another system known in the art is described in U.S. Pat. No. 9,011,410 (hereinafter, “the '410 patent”), which is incorporated herein by reference in its entirety. The '410 patent describes a drug or cell delivery system for multi-segmental injection of cells and/or therapeutic substances into the spinal cord of an animal or a human. The device provides for delivery of a substrate into a spinal cord and comprises a guide needle having an inside diameter; an injection needle fitting into the inside diameter of the guide needle; a stepping motor advancing the injection needle into and within the spinal cord; and a chamber containing the substrate or cells in fluid communication with the injection needle. In operation, the device may deliver a substrate into a spinal cord. The administration method comprises advancing a guide needle into the spinal cord, the guide needle having a bend at an angle of about 45 degrees at an end thereof, the end being advanced into the spinal cord; advancing an injection needle through the guide needle and into the spinal cord with a stepping motor attached to the injection needle; and then injecting the substrate into the spinal cord through a syringe attached to the injection needle. The external end of the injection needle is directly connected to the syringe with polyethylene tubing. When the injection needle is withdrawn, the cells and/or other therapeutic substance may be injected into the spinal cord. The stepping motor attached to the injection needle between the syringe and a portion of the injection needle inside the guide needle may provide for multi- segmental delivery of the cells and/or other therapeutic substance into the spinal cord. The foregoing apparatus requires a fixed bend to the guide needle into the spinal cord that in certain instances impedes the positioning of the injection needle within the spinal cord and therefore may impair the deposition of a longitudinal trail of cells and/or other therapeutic substances within the spinal cord parenchyma.

As discussed above, the '410 patent discloses a device and method for multi-segmental delivery of a substrate into the spinal cord employing a bent guide needle, linear actuator controlled injection needle, and syringe. An important component of the '410 patent is the described guide needle and associated method. This guide needle has a 45 degree bend at the tip and is inserted at a 45 degree angle in relation to the cord. Inserting such a needle into the spinal cord might cause substantial damage to the spinal cord. Furthermore, there is no way to control the angle of the resulting therapeutic trail. Differing patient anatomies, pathologies, and therapeutic mechanisms may require alternate angles of trails within the spinal cord. The ability to create two trails that meet at a vertex, like a tent, may also be of therapeutic benefit and is not possible with the device\method described in '410 patent. Moreover, the described device states that a stepping motor is attached to the injection needle between the syringe and a portion of the injection needle. This arrangement alone does not enable insertion of an injection needle into the spinal cord because the injection needle may buckle between the guide needle and linear actuator attachment. Another disadvantage of the disclosed method is the polyethylene tubing used to connect the injection needle and the syringe. This flexible polyethylene tubing increases the dead volume between the syringe and the tip of the injection needle, potentially resulting in loss of therapeutic and reduced control of the injection flow rate and delivery volume.

Another system known in the art is described in U.S. Pat. No. 9,192,408 (hereinafter, “the '408 patent”), which is incorporated herein by reference in its entirety. The '408 patent describes a system and method that is directed to medical treatments of organs having anatomical spaces, such as the heart and the pericardial space. The methods and apparatus may include a first elongated member with a sharp tip used to penetrate the surface surrounding the anatomical space with a second elongated member with a helical tine used to engage the surface and lift the surface away from the underlying anatomical space. Once the first elongated member has incised the surface, it is removed, and the incision may be used as a point of entry for delivering media or medical devices into the anatomical space, or for carrying out further medical procedures. Thus, the '408 patent describes a surgical intervention apparatus and method which may not be suitable to administration and delivery of a long trail of therapeutic cells and/or at least one therapeutic substance, or diagnostic substance or other injectable medium into the narrow diameter (generally on the order of <1 to 1.5 cm) of the spinal cord, or other like anatomical space, where destruction of adjacent tissue is neither desired nor intended.

### BRIEF SUMMARY OF THE INVENTION

The foregoing injection problems identified in the art are addressed by the injection device and system described in this specification and appended drawings. The injection system and methods of the present invention are capable of depositing trails of therapeutic cells that may cross an injury of an anatomical space, for instance the spinal cord, between two points along the longitudinal axis of the anatomical space, i.e. the spinal cord. The two points may be rostral and caudal to an injury site of the cord and may be due to a compression or contusion injury or the severance or partial severance of the spinal cord. For conditions such as ALS, the trail may not cross an injury site per se, but rather the injection of a trail of therapeutic cells and/or at least one therapeutic substance or diagnostic substance, or other injectable medium, may enable the continuous application of therapeutic cells into a diseased cord without multiple puncture sites for the purpose of cellular therapy, including somatic cellular therapy and gene therapy. For instance, in treating ALS, the trail of cells may be positioned near the ventral horn motor neurons. The same would be true with respect to MS, where remyelination of the axons of diseased spinal cord neurons may be an objective of the injection of a trail of therapeutic cells and/or at least one therapeutic substance or diagnostic substance, or other injectable medium. The same would also hold true for other ischemic and pathological conditions of the spinal cord. In the foregoing treatments, one of the principal objectives, therefore, is to minimize the number of penetrations into the spinal cord parenchyma. A subpial trail location could be used for gene therapy (for example for ALS or spinal muscular atrophy) or cell delivery.

### Injectable Medium

An injectable medium useful in the injection system of the present invention is described in co-pending non-provisional patent application, application Ser. No. ______, filed on the same date herewith and entitled COMPOSITIONS AND METHODS FOR PREPARING AN INJECTABLE MEDIUM FOR ADMINISTRATION INTO THE CENTRAL NERVOUS SYSTEM, the entire contents of which is hereby incorporated by reference.

Injectable media comprising therapeutic cells, and optionally therapeutic or diagnostic substances, in particular neural stem cells, and hyaluronic acid, have been found to prevent cell settling during transportation and storage of such injectable media of therapeutic cells, and optionally therapeutic or diagnostic substances. Such injectable media also promote cell survival, facilitate administration of homogeneous therapeutic cell suspensions, in particular homogenous NSC suspensions, and enable rapid clearance by the body following injection so as not to interfere with cellular integration with surrounding tissue.

Importantly, combining the therapeutic cells with the carrier should not adversely affect cell viability during mixing, or upon injection or at the transplantation site.

Sedimentation of therapeutic cells, such as neural stem cells, due to cellular aggregation may occur in storage solutions for therapeutic cells, therapeutic cell delivery systems and therapeutic cell delivery compositions, as described previously herein. Moreover, the sedimentation of cells may occur almost instantaneously after injection, with the cells rapidly advancing down an angled injection trail to be deposited in an undesirable mass.

The injectable media described herein enable the preparation of storage stable liquid compositions of suspended therapeutic cells, and optionally therapeutic or diagnostic substances, for the manufacture, storage and delivery of therapeutic cells to a target delivery site, i.e. an anatomical space within the body of a human or an animal subject, particularly in the CNS, and especially the spinal cord, of a subject, in various diagnostic and therapeutic settings.

Injectable compositions comprising hyaluronic acid (“HA”) and therapeutic cells, and optionally therapeutic or diagnostic substances are useful in applications where there exists a need for delivery of uniform suspensions comprising hyaluronic acid, and viable populations of therapeutic cells, and optionally therapeutic or diagnostic substances, for purposes of cell transplantation and cell therapy into a site of injury within an anatomical space of the body, in particular injections into the central nervous system (“CNS”) including the brain and, most preferably, injections directly into the spinal cord. Such compositions provide for delivery of viable populations of therapeutic cells, and optionally therapeutic or diagnostic substances to enhance the survival, differentiation and integration of transplanted cells into the body, including the CNS and the spinal cord of a human or animal subject.

Cell delivery and the subsequent survival of transplanted cells are significant problems to be solved to provide for successful cellular transplantation. Most transplanted cells frequently die or migrate away from the transplant site and/or aggregate together. The result is that transplanted cells may not integrate with the host tissue. Klassen, H. J., Ng, T. F., Kurimoto, Y., Kirov, I., Shatos, M,. Coffey, P. et al., “Multipotent retinal progenitors express developmental markers, differentiate into retinal neurons, and preserve light-mediated behavior,” Invest Ophthalmol Vis. Sci., 45(11):4167-73 (2004); Potts, M. B., et al., “Devices for cell transplantation into the central nervous system: Design considerations and emerging technologies,” Surg. Neurol Int., 4(Suppl) S-22-S30 (2013).

Successful implementation of cellular therapy requires cell survival, appropriate cell distribution, and implanted cell integration with tissue. Furthermore, translational concerns such as stability during transportation and administration must be addressed. When examining the delivery of cells such as neural stem cells (NSCs) for treatment of pathologies such as spinal cord injury, the cells should be delivered in a minimally invasive fashion (injectable) and differentiate into appropriate regenerative lineages (i.e. neurons, astrocytes, and oligodendrocytes). The requirements listed above may be addressed, in part, by selecting an appropriate carrier for the cell therapy as described herein.

The design requirements for an acceptable cell carrier are to: 1) prevent cell settling during transportation and injection storage, 2) promote cell survival, 3) facilitate administration of homogeneous therapeutic cell suspensions, and 4) enable rapid clearance by the body following injection so as not to interfere with cellular integration with surrounding tissue. Importantly, combining the cells with the carrier should not adversely affect cell viability during mixing, or upon injection or at the transplantation site.

Sedimentation of therapeutic cells due to cellular aggregation occurs in storage solutions for stem cells, stem cell delivery systems and stem cell delivery compositions. The present invention enables the preparation of storage stable liquid compositions of suspended stem cells for the manufacture, storage and delivery of stem cells to a target delivery site in the spinal cord of a human or animal in various diagnostic and therapeutic settings.

The present invention describes liquid compositions comprising, for example, human neural stem cells suspended in a liquid medium that offers numerous advantageous properties by preventing cellular aggregation and minimizing the disruption and sedimentation of stem cells during transport, storage and administration of such liquid compositions. The liquid compositions comprising human neural stem cells are suitable for the delivery of the human neural stem cells to the CNS, particularly to the spinal cord, of a human or animal subject, in various diagnostic and therapeutic settings. The present invention is also suitable in applications such as cell therapy and tissue engineering (such as 3-D printed cellular constructs).

While the delivery and administration of human neural stem cells is a preferred embodiment of the present invention, other types of therapeutic cells may be administered using the methods and compositions described herein. Therapeutic cells may also include neural stem cells, pre-differentiated cells in the neuronal lineage, glial cells, glial restricted progenitor cells, Schwann cells, olfactory ensheathing cells, fibroblasts, mesenchymal stem cells, adipose derived stem cells, induced pluripotent stem cells, embryonic stem cells, bone marrow derived stem cells, hematopoietic stem cells, the differentiated progeny of any of the above, genetically modified cells, or other cell types.

The described injectable media may also be utilized to deliver therapeutic substances, alone, or more preferably together with therapeutic cells to the CNS, especially to the spinal cord. Various neurotropic factors are contemplated in the art. Therapeutic agents that may be incorporated into the liquid composition comprising hyaluronic acid include: Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other like therapeutic agents.

The administration of trophic and growth factors such as erythropoietin (EPO), brain-derived neurotrophic factor (BDNF), nerve growth factor (NGF), fibroblast growth factor (FGF) and epidermal growth factor (EGF) appear to play an important role in in-vitro and in-vivo survival and differentiation of stem cells (Erickson et al., Roles of insulin and transferrin in neural progenitor survival and proliferation. J. Neurosci. Res. 2008 Feb. 21; Bossolasco et al., Neuro-glial differentiation of human bone marrow stem cells in vitro., Exp. Neural., 2005 June; 193(2):312-25). Nerve growth factor (NGF) appears to influence grafted tissue in the CNS. Mahoney. et al. (1999). Med. Sci. 96:4536-4539.

Other regulatory agents comprising various growth factors including insulin-like growth factor-I (IGF-I) and basic fibroblast growth factor (bFGF) also regulate the survival and differentiation of nerve cells during the development of the peripheral and central nervous systems. IGF-I promotes differentiation of post-mitotic mammalian CNS neuronal stem cells. Arsenijevic, et al. (1998) J. Neurosci. 18:2118-2128. Similarly, neurotrophins have been shown to be important for nerve growth during development. Tucker, et al. (2001), Nature Neurosci., 4:29-37). GAP-43 and CAP-23 act to promote regeneration of injured axons and may support regeneration in the spinal cord and CNS. Bomze et al. (2001) Nature Neurosci. 4:38-43 and Woolf et al. (2001) Nature Neurosci. 4:7-9. Cocktails of growth factors may be used to further increase cell survival, neuronal differentiation, axon extension, and synapse formation (Lu, et al., Long-Distance Growth and Connectivity of Neural Stem Cells after Severe Spinal Cord Injury. Cell, 2012, Sep. 14; 150(6): 1264-1273).

Given the various problems of delivering a trail of neural stem cells into the spinal cord parenchyma or beneath the pia matter of a spinal cord by administration and delivery systems known in the art, there is still a need for injectable media comprising human neural stem cells suspended in a liquid medium that offers numerous advantageous properties by preventing cellular aggregation and minimizing the disruption and sedimentation of stem cells during transport, storage and administration of such injectable media.

Unique advantages provided by the injectable media described herein include improved properties of the cellular suspensions after a prolonged storage period, improved cellular homogeneity during and after injection, improved clearance from the central nervous system in a comparably short amount of time after injection, and potentially the facilitation of the suspended stem cells to interact via receptors on the neural stem cell surface to promote cell survival after storage and\or injection of the compositions of the invention.

In a first aspect, the injectable medium comprises therapeutic cells, and optionally therapeutic or diagnostic substances, suitable for injection into an anatomical space of a human or animal subject, comprising:

(a) therapeutic cells, and optionally therapeutic or diagnostic substances;

(b) a pharmaceutically acceptable diluent comprising hyaluronic acid;

wherein the injectable medium has a storage modulus within the range of 5-25 Pa.

In a second aspect, the injectable medium comprises therapeutic cells, and optionally therapeutic or diagnostic substances, suitable for injection into an anatomical space of a human or animal subject, wherein

(a) therapeutic cells, and optionally therapeutic or diagnostic substances;

(b) a pharmaceutically acceptable diluent comprising hyaluronic acid;

wherein the hyaluronic acid is formulated at a concentration of about 0.5 wt. % to 1 wt. % in the injectable medium; and further wherein the hyaluronic acid has a molecular weight of about ≧700 kDa to about 1,900 kDa; and wherein the injectable medium has a storage modulus within the range of 5-25 Pa.

In a third aspect, the injectable medium comprises neural stem cells, and optionally therapeutic or diagnostic substances, suitable for injection into an anatomical space of a human or animal subject, comprising:

(a) human neural stem cells, and optionally therapeutic or diagnostic substances;

(b) a pharmaceutically acceptable diluent comprising hyaluronic acid;

wherein the injectable medium has a storage modulus within the range of 5-25 Pa.

In a fourth aspect, the injectable medium comprises neural stem cells, and optionally therapeutic or diagnostic substances, suitable for injection into an anatomical space of a human or animal subject, comprising:

(a) human neural stem cells, and optionally therapeutic or diagnostic substances;

(b) a pharmaceutically acceptable diluent comprising hyaluronic acid;

wherein the hyaluronic acid is formulated at a concentration of about 0.5 wt. % to 1 wt. % in the injectable medium; and further wherein the hyaluronic acid has a molecular weight of about ≧700 kDa to about 1,900 kDa; and wherein the injectable medium has a storage modulus within the range of 5-25 Pa.

In a fifth aspect, the injectable medium is prepared by a method of preparing an injectable medium comprising therapeutic cells, and optionally one or more therapeutic or diagnostic substance, suitable for injection into an anatomical space of a human or animal subject, comprising the steps of:

(a) introducing into a sterilized vial a desired quantity of therapeutic cells, and optionally one or more therapeutic or diagnostic substance;

(b) adding to the vial a pharmaceutically acceptable diluent comprising hyaluronic acid;

(c) mixing the above composition until a substantially uniform suspension is obtained having a storage modulus within the range of 5-25 Pa.

In a sixth aspect, the injectable medium is prepared by a method of preparing an injectable medium comprising therapeutic cells, and optionally one or more therapeutic or diagnostic substance, suitable for injection into an anatomical space of a human or animal subject, comprising the steps of:

(a) introducing into a sterilized vial a desired quantity of therapeutic cells, and optionally one or more therapeutic or diagnostic substance;

(b) adding to the vial a pharmaceutically acceptable diluent comprising hyaluronic acid;

(c) resuspending the therapeutic cells in the diluent by agitating the vial;

wherein the hyaluronic acid is formulated at a concentration of about 0.5 wt. % to 1 wt. % in the injectable medium; and further wherein the hyaluronic acid has a molecular weight of about ≧700 kDa to about 1,900 kDa;

(d) mixing the above composition until a substantially uniform suspension is obtained having a storage modulus within the range of 5-25 Pa.

In a seventh aspect, the present invention is prepared by a method of preparing an injectable medium comprising neural stem cells, and optionally therapeutic or diagnostic substances suitable for injection into an anatomical space of a human or animal subject, comprising the steps of:

(a) introducing into a sterilized vial a desired quantity of human neural stem cells;

(b) adding to the vial a pharmaceutically acceptable diluent comprising hyaluronic acid;

(c) mixing the above composition until a substantially uniform suspension is obtained having a storage modulus within the range of 5-25 Pa.

In an eighth aspect, the injectable medium is prepared by a method of preparing an injectable medium comprising neural stem cells, and optionally therapeutic or diagnostic substances suitable for injection into an anatomical space of a human or animal subject, comprising the steps of:

(a) introducing into a sterilized vial a desired quantity of human neural stem cells;

(b) adding to the vial a pharmaceutically acceptable diluent comprising hyaluronic acid;

(c) resuspending the human neural stem cells in the diluent by agitating the vial;

wherein the hyaluronic acid is formulated at a concentration of about 0.5 wt. % to 1 wt. % in the injectable medium; and

further wherein the hyaluronic acid has a molecular weight of about ≧700 kDa to about 1,900 kDa;

(d) mixing the above composition until a substantially uniform suspension is obtained having a storage modulus within the range of 5-25 Pa.

In a ninth aspect the injectable medium comprises the therapeutic cells of the first, second, fifth and sixth aspects which include pre-differentiated cells in the neuronal lineage, glial cells, glial restricted progenitor cells, Schwann cells, olfactory ensheathing cells, fibroblasts, mesenchymal stem cells, adipose derived stem cells, induced pluripotent stem cells, embryonic stem cells, bone marrow derived stem cells, hematopoietic stem, genetically modified cells, neural precursor cells of the forebrain, midbrain, hindbrain, spinal cord, neural crest, and retinal precursors isolated from developing tissue, and the undifferentiated and differentiated progeny of any of the above.

In a tenth aspect the injectable medium comprises the human neural stem cells of the third, fourth, seventh and eight aspects which may be undifferentiated or differentiated cells.

In an eleventh aspect the injectable medium comprises, the cells of the first to tenth aspects which may be delivered as spheres, aggregates or single cell suspensions.

In a twelfth aspect the injectable medium comprises the pharmaceutically acceptable diluent of the first to eleventh aspect may be divalent ion-free buffed salt solution; phosphate buffered saline; cell culture medium, isotonic saline, hanks buffered salt solution, HEPES buffered salt solution, and artificial cerebrospinal fluid.

In a thirteenth aspect, the injectable medium comprises the pharmaceutically acceptable diluent of the first to twelfth aspects which may further comprise ascorbic acid, glucose, or glutamine.

In a fourteenth aspect, the injectable medium comprises the injectable medium of the first to thirteenth aspects which may further comprise a neuroprotective, angiogenic, anti-angiogenic or neuroregenerative pharmaceutical substance.

In an fifteenth aspect, the injectable medium comprises the injectable medium of the first to fourteenth aspects which may further comprise at least one factor capable of stimulating endogenous stem cells.

In a sixteenth aspect, the injectable medium comprises the injectable medium of the first to fifteenth aspects which may further comprise a drug and/or growth factor selected from the group consisting of: Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other therapeutic agents.

In a seventeenth aspect, the injectable medium comprises the injectable medium of any of the preceding aspects may further comprise a regulatory agent, as described herein.

In an eighteenth aspect, the injectable medium comprises the injectable medium of any of the preceding aspects which may further comprise a therapeutic substance, as described herein.

In a nineteenth aspect, the injectable medium comprises the injectable medium of any of the preceding aspects which may be injected into the spinal cord of a subject using a suitable injection system that deposits one or more trails of therapeutic cells, including human neural stem cells within the spinal cord of the subject.

In a twentieth aspect, the injectable medium comprises the injectable medium of any of the preceding aspects which may be injected into the brain of a subject using a suitable injection system that deposits one or more trails of therapeutic cells, including human neural stem cells within the brain of the subject

In yet another aspect, the injectable medium is administered to a human or animal subject by a method of injecting one or more trails of neural stem cells within the spinal cord of the subject to treat a spinal cord injury, condition or disease.

In still yet another aspect, the injectable medium comprises human neural stem cells suspended in a carrier comprising high molecular weight hyaluronic acid at a concentration of about 0.5 weight % to about 1 weight % in the injectable medium; wherein the hyaluronic acid has a molecular weight of about ≧700 kDa to about 1,900 kDa; and wherein the composition enables the human neural stem cells to be suspended uniformly for up to two days, up to three days, up to four days or up to five days.

In a further aspect the injectable medium may comprise a kit suitable for injecting a trail of neural stem cells into the spinal cord of a subject using a suitable injection system, wherein the kit comprises hyaluronic acid at a concentration of 0.5 weight % to 1 weight % in an injectable medium wherein the hyaluronic acid has a molecular weight of about ≧700 kDa to about 1,900 kDa; and wherein the composition enables the human neural stem cells to be suspended uniformly for up to two days, up to three days, up to four days or up to five days.

### Gene Therapy Applications

Gene therapy involves a medical intervention that transiently or permanently modifies the genetic material of living cells. The modification may involve adding, subtracting or replacing genetic information. The genetic manipulation may be intended to have a therapeutic or prophylactic effect in the subject receiving gene therapy. Vectors (e.g. viruses, liposomes), additives (e.g. polybrene), recombinant RNA or DNA materials used to modify the genetic material of cells are considered components of gene therapy. The genetic material may encode a product or products (e.g., enzyme, protein, polypeptide, peptide, non-coding RNA, coding RNA) or regulate the expression of a gene product (e.g. enhance or repress). The gene product may encode a hormone, receptor, enzyme, polypeptide, peptide, interfering RNA, targeted gene editing products (e.g. meganucleases, zinc finger nucleases (ZFNs), transcription activator-like effector-based nucleases (TALEN) or CRISPR-Cas9) of therapeutic value. For a review see “Gene and Cell Therapy: Therapeutic Mechanisms and Strategies, Fourth Edition” Nancy Smyth Templeton (2015).

Gene therapy is divided into two types of therapy: ex vivo and in vivo. Ex vivo gene therapy involves genetic modification of cells from a subject or a donor outside the body, which are then transplanted into a subject. In vivo gene therapy involves genetic modification of cells within a subject's body. Gene therapy can be performed without a vector (e.g. naked DNA, electroporation, gene gun, sonoporation, magnetofection, hydrodynamic) or with vector (e.g. viral or chemical). Various viral vectors are known in the art, which include retroviruses or adenoviruses, adeno-associated viruses, lentiviruses, pox viruses, alphaviruses and herpes viruses. Various chemical vectors are known in the art, which include lipoplexes, polymersomes, polyplexes, dendrimers, inorganic nanoparticles and cell penetrating peptides. For a review see “Non-viral vectors for based therapy” Yin et al. (2014) Nature Reviews Genetics 15; 541-55 and “Gene therapy returns to centre stage” Naldini (2015) Nature 526; 351-60.

Administration of gene therapy may be performed through, for example, intravenous injection using a vector capable of crossing the blood brain barrier or by spinal tap into the cerebrospinal fluid surrounding the brain and spinal cord. The gene may be delivered in a modified virus that carries the genes to cells in the subject's body. An example would be the clinical trial described as “Intrathecal Administration of scAAV9/JeT-GAN for the Treatment of Giant Axonal Neuropathy,” as Trial No. NCT02362438 at www. Clinical Trials.gov.

Alternatively, the gene of interest may be transferred by infusion following a surgical procedure to infuse the viral vector and gene into the brain of a subject. An example would be to treat Parkinson's disease, such as the clinical trial described as “Phase 1 Open-Label Dose Escalation Safety Study of Convection Enhanced Delivery (CED) of Adeno-Associated Virus Encoding Glial Cell Line-Derived Neurotrophic Factor (AAV2-GDNF) in Subjects with Advanced Parkinson's Disease,” identified as Study NCT01611581 at www.ClinicalTrials.gov. See also, Bjorklund A, Kirik D, Rosenblad C, Georgievska B, Lundberg C, Mandel R J. Towards a neuroprotective gene therapy for Parkinson's disease: use of adenovirus, AAV and lentivirus vectors for gene transfer of GDNF to the nigrostriatal system in the rat Parkinson model. Brain Res. 2000 Dec. 15; 886(1-2):82-98. Review.

Another exemplary trial is NCT01973543, entitled “An Open-label Safety and Efficacy Study of VY-AADC01 Administered by MM-Guided Convective Infusion Into the Putamen of Subjects With Parkinson's Disease With Fluctuating Responses to Levodopa. In the latter study, a hAADC gene is packaged into a gene transfer vector derived from a common, non-pathogenic virus (AAV2) to which >90% of humans have been exposed. The investigational drug, termed VY-AADC01, will be injected directly into the striatum during a neurosurgical procedure that is performed with real-time MM imaging to monitor delivery.

Additional references. Experimental Eye Research 89; 301-310. Bible E, Chau, Y S, Alexander M R, Price J, Shakesheff K R, Modo M. (2009) The support of neural stem cells transplanted into stroke-induced brain cavities by PLGA particles. Discovery Medicine 15; 111-9. Nagabhushan Kalburgi S, Khan N N, Gray S J. (2013) Recent gene therapy advancements for neurological diseases. Human Gene Therapy 27; 478-96. Hocquemiller M, Giersch L, Audrain M, Parker S, Cartier N (2016) Adeno-Associated Virus-Based Gene Therapy for CNS Diseases. Various publications, including patents, published applications, technical articles and scholarly articles are cited throughout the specification. Each of these cited publications is incorporated by reference herein, in its entirety.

In some embodiments of the invention, the injection of one or more trails of therapeutic cells and/or at least one therapeutic substance or diagnostic substance, or other injectable medium, may surround the injury site by angular injections, for instance, spanning the grey to white matter proximal to the injury site. In other embodiments, a trail of therapeutic cells and/or at least one therapeutic substance or diagnostic substance, or other injectable medium injected parallel to the longitudinal axis of the spinal cord, spanning grey to grey matter or white to white matter, may be created as well. This may be accomplished by creating a small incision (myelotomy) in the spinal cord, inserting the guide needle into the spinal cord parenchyma, and extruding the injection needle parallel to the spinal cord.

In some embodiments of the present invention, a flexible injection catheter is inserted under the pia of the cord and extruded parallel to the cord. Therapeutics such as cells or gene therapy agents are deposited at maximal extension of the injection catheter and/or along the trail created during retraction of the catheter.

In some embodiments of the present invention, angular injections are made proximal to the injury site, thereby depositing one or more trails of therapeutic cells and/or at least one therapeutic substance or diagnostic substance, or other injectable medium in the form of a “tent” as depicted in the Figures accompanying Example 1. By depositing a trail of therapeutic cells, severed or diseased axons and/or severed and/or diseased neurons may be connected through regeneration of neurons at the injury site. The trails of cells may be defined by the path of the injection needle advancing and retracting through or around the injury site in the spinal cord. Reference may be made to figures accompany this specification, in particular to FIG. 51, which depicts cell trails injected in an in vitro model. In addition, based on diagnostic imaging techniques known to the skilled artisan, such as magnetic resonance imaging (“MRI scan”), computed tomography (“x-ray CT scan”), fluoroscopy, computerized axial tomography (“CAT scan”) and position emission tomography (“PET scan”) and other diagnostic imaging techniques, the preferred geometry of an injection or multiple injections of cells and/or a therapeutic substance may be determined and then be administered using an embodiment of the present invention.

Among other aspects, the injection system employs a curved guide needle (sometimes alternatively referred to herein as an “introducer needle” or “guide tube”), which is positioned on the surface of the cord (specifically, the pia) and guides the entry of a delivery catheter , for example, an elastic, flexible wire or synthetic polymeric delivery catheter (referred to interchangeably as an “injection” or “delivery catheter” or sometimes “needle”) into the spinal cord parenchyma or, alternatively, on the surface of the spinal cord. The delivery catheter may be blunt or bear a needle point or other geometry to enable entry into a desired anatomical space, for instance the spinal cord. In a preferred embodiment, the injection needle/delivery catheter is made from a nitinol (nickel-titanium alloy) flexible cannula having a needle bevel at one end. In an alternative embodiment, the delivery catheter may be fabricated from a material comprising a synthetic or natural polymer, for example, a polyester such as polyethylene. The delivery catheter/injection needle is extruded to a specified distance and then retracted while a syringe having a mechanized plunger rod assembly flows the trail of therapeutic cells and/or at least one therapeutic substance or diagnostic substance, or other injectable medium out of the delivery catheter/injection needle. The injection procedure results in the creation of a trail of therapeutic cells and/or at least one therapeutic substance or diagnostic substance, or other injectable medium, within the spinal cord. Substantial control over the penetration angle into the spinal cord is achieved by embodiments of the present invention. The foregoing may be readily visualized with reference to Example 3 and FIGS. 53-57, which are described in greater detail below. Moreover, the injection procedure is minimally invasive with respect to penetration of the spinal cord parenchyma. The pia is nicked with a needle at the penetration site and a flexible needle apparatus, preferably a nitinol (nickel-titanium alloy) flexible cannula in a preferred embodiment, is introduced at a controlled entry angle. In a specific embodiment, the trail of therapeutic cells and/or at least one therapeutic substance or diagnostic substance, or other injectable medium may be administered below the pia without entering the spinal cord parenchyma. Flexible catheters of preferably as small as 29 gauge (or smaller) may be utilized. Injection rates and volumes can be very closely controlled by a programmed controller, as described below. Cell trails of therapeutic cells and/or at least one therapeutic substance or diagnostic substance, or other injectable medium may desirably be introduced at angles resulting in deposition of a trail extending in a caudal to rostral or rostral to caudal direction. Injection cell trails may also be deposited at angles spanning gray to white matter in the spinal cord parenchyma.

In an embodiment, the present invention addresses significant problems inherent in the '177 and '410 patents. The present invention, in an embodiment, avoids the use of an endoscope-based approach and automates the needle insertion and fluid delivery. This enables a simplified surgical approach with reproducible therapeutic trail positioning and delivery. The present invention also addresses problems in the prior art apparatus utilizing linear actuators.

### Linear Actuator

In the present invention, a linear actuator may be utilized to provide linear movement of the delivery catheter (also identified as an injection needle) to extend and retract the delivery catheter in the desired anatomical space. Similarly, a linear actuator may provide linear motion to the plunger rod of the syringe to eject the contents of the syringe. A preferred embodiment of the linear actuator is a stepper motor. In some embodiments, as noted herein, a rotary friction drive can provide linear motion to the delivery needle and is subsumed within the term linear actuator.

The movement of the delivery catheter/injection needle may be accomplished by means of a linear actuator. The same is true for movement of the plunger rod of the syringe for the purpose of ejecting the contents of the syringe, or, if reversed, to aspirate a fluid from an anatomical pace in the body of a subject.

A “linear actuator” is a mechanism for the conversion of energy into linear motion. In the context of the present invention, non-limiting examples of a linear actuator may comprise a linear actuator, brushless servo motor, brushed servo motor, a lead screw. a ball screw, a rack and pinion mechanism, a Scotch yoke, a belt and pulley drive, a chain drive, or any other mechanism that converts rotary motion into linear motion. The foregoing definition also subsumes use of a rotary friction drive adapted to provide linear movement to the delivery catheter/injection needle as an equivalent to a linear actuator.

The skilled person will understand that the function of a mechanized linear actuator can be achieved by a manual hand motion, for example in extruding the contents of syringe and/or extending and retracting the delivery catheter. Such manual actuation is within the scope of the present invention disclosed herein.

The skilled person will understand that a conventional linear actuator may be utilized as a linear actuator (i) to advance and retract the delivery catheter, also referred to as the injection needle) and (ii) to control the volume and flow rate of the contents of the pre-filled syringe through actuation of the plunger rod in the operation of the injection device.

A specific example of a linear actuator used in a preferred embodiment is an E28M4AC-2.1-A01, Haydon Kerk.

The linear actuator may be operated through a programmable controller as described herein.

In a preferred embodiment, the operation of the linear actuator is connected to a motor driver (R525P, Lin Engineering) which is controlled by an Arduino MEGA 2560 (Arduino). Rotation of the linear actuator drives a linear rail (RGS04, Haydon Kerk) which actuates the position of the injection needle and/or syringe plunger. The skilled person will understand that the various linear actuator mechanisms described above may provide equivalent translation of rotary motion into linear motion.

The source of energy for the linear actuator is electrical energy in a preferred embodiment.

Equivalent mechanisms power from air or a liquid may also be adapted in alternative embodiments.

### Guide Tube/Introducer Needle

In other embodiments, the present invention discloses novel improvements to the guide tube (introducer needle) design, employing a curved instead of 45 degree bent needle as shown in the art, as well as providing for precise and adjustable positioning and angle mechanisms. In an embodiment, the present invention utilizes a telescoping two-part cannula injection needle drive mechanism (sometime referred to as a “trombone” sliding tube mechanism herein) to prevent buckling of the injection needle (see FIG. 33). This enables accurate extrusion of the injection needle.

The motorized injection flexible needle (catheter) wire may preferably be fabricated from nitinol memory wire catheter manufactured with a nickel-titanium typically comprising almost equal atomic weight percentages of nickel and titanium, which have been approved for surgical use. Alternatively, the delivery catheter may be fabricated from a synthetic or natural polymeric material or a co-polymeric substance.

### Goniometer

Also, in an embodiment, the present invention employs a goniometer to precisely adjust the angle of trail creation and the rotational axis about the tip of the guide needle. In some embodiments, the goniometer works together with the rotation stage of the micro-adjustment apparatus to control the x, y, z orientation of the distal end of the delivery catheter. This configuration permits creation of “tent” trail geometries among other injection trail geometries (see FIG. 34C).

Snap-on connectors of various designs and materials (946) as will be apparent to the skilled worker may be utilized to secure the delivery catheter (943) \trombone assembly (945a, 945b) to the injector device subassembly (940) described herein as well as one or more linear actuators, for example, linear actuators (959a, 959b). Exemplary snap-on connectors are depicted and described in the specification and figures (e.g., FIG. 19). The snap-on connectors (946) allow for the use of a disposable delivery catheter/injection needle 943\guide tube (needle) 942 assembly (FIG. 49), for sterility purposes. The snap-on connectors also facilitate and enable the trombone mechanism (945a, 945b), by connecting the articulating trombone 945a and b delivery catheter (FIG. 31)\delivery catheter/injection needle 943 component to the linear actuator 959a (see FIG. 59).

Polyethylene tubing may be eliminated by extending the length of the injection needle such that an injection needle service loop 944 (see FIG. 60) is formed between the linear actuator 959b connected to the syringe plunger rod 941c. This “service loop” (944) bends forward as the injection needle is inserted into the spinal cord and bends back when it is retracted. Without a service loop, the injection needle might break at its connection with the syringe during motion.

The foregoing exemplary embodiments afford precise control of the injected fluid path, by minimizing or eliminating the use of flexible polyethylene tubing, thereby resulting in improved flow profile and less fluid loss.

An important advantage conferred by the disclosed configuration of embodiments of the disclosed injection device is that in surgical settings the guide tube (introducer needle) may be positioned on the surface of pia (rather than being inserted into the spinal cord parenchyma), with penetration of the spinal cord parenchymal cells only by the narrower gauge injection needle (943). This disclosed configuration also allows the guide tube to be positioned such that a flexible catheter is extruded under the pia, preventing potential damage of the spinal cord parenchyma.

Embodiments of the present invention disclosed in the specification and drawings enable the health care practitioner to determine trail angles accurately based on the pathology exhibited by an individual subject, and to adjust the angle of entry of the injection needle into the anatomical space, for instance the spinal cord parenchyma, at the time of surgery.

Embodiments of the present invention provide an improved method and apparatus for delivering trails of therapeutic cells and/or at least one therapeutic substance or diagnostic substance, or other injectable medium either directly into the spinal cord of an animal, particularly, a human subject, or administered by subpial injection, as described herein. In another embodiment, the injection system may be utilized to remove fluids from an anatomical space to alleviate the sequelae of trauma or disease to such anatomical space. In a first aspect of the present invention, an injection system for delivering an injectable medium into an anatomical space of an animal or human subject, for instance, a trail of therapeutic cells and/or at least one therapeutic substance or diagnostic substance, or other injectable medium . The anatomical space may be, for instance, a brain or a spinal cord, or an adjoining tissue such as by subpial injection.

The injection system for delivering an injectable medium into an anatomical space of an animal or human subject, in a first aspect comprises

a first linear actuator;

a syringe comprising a catheter connection at one end and a plunger attached to a plunger rod at a second end, wherein the syringe contains an injectable medium for injection into an anatomical space of an animal or human subject;

a delivery catheter having a proximal and distal end, wherein the distal end is configured to enter the anatomical space of a subject, and wherein the proximal end is attached to the catheter connection of the syringe; a guide tube having a proximal end and a distal end, wherein the guide tube is configured to house a portion of the distal end of the delivery catheter; further wherein the proximal end of the guide tube is connected to a guide tube holder;

a stereotaxic assembly connected to the guide tube holder, a stereotaxic assembly connected to the guide tube holder, thereby allowing spatial adjustments along the x, y and z- axes; wherein the stereotaxic positioning assembly is configured to move the distal end of the guide tube in spatial alignment with the external surface of the spinal cord of a subject and allows rotation about the x, y, and z axes to control the orientation of the guide tube;

wherein the delivery catheter engages the linear actuator along the length of the catheter;

wherein the distal end of the guide tube is formed in a bend relative to the proximal end of the guide tube; and wherein the first linear actuator is configured to extend and retract the delivery catheter inside the guide tube.

### Stereotaxic Assembly

A stereotaxic assembly 204 allows spatial adjustments along the x, y and z-axes. The stereotaxic assembly is configured to move the distal end of the guide tube in spatial alignment with the external surface of the spinal cord of a subject and allows rotation about the x, y, and z axes to control the orientation of the guide tube. In some embodiments of the stereotaxic assembly is identified interchangeably as an XYZ mounting system 915. The XYZ mounting system, as disclosed in the specification, provides very precise positioning of the guide tube/needle and delivery catheter/injection needle relative to the orientation of the spinal cord parenchyma of the subject. This is particularly true in a preferred embodiment when a goniometer pitch adjustment is utilized. Numerous embodiments of the stereotaxic/XYZ mounting system are discussed throughout the specification and depicted in the Figures.

In a second aspect, the injection system according to the first aspect, features a guide tube may comprise a (i) distal guide tube having a distal and proximal end, and (ii) a tubing having a distal and proximal end; wherein the distal end of the distal tube is formed in a bend relative to the proximal end of the distal guide tube; wherein the distal guide tube is joined to the guide tube holder; further wherein the proximal end of the distal guide tube is connected to the distal end of the tubing, and wherein the proximal end of the guide tube is connected to an attachment to the first linear actuator; wherein the (i) distal guide tube and the (ii) tubing house a portion of the distal end of the flexible delivery catheter

In a third aspect, the proximal end of the delivery catheter is connected to the catheter connection of the syringe by tubing.

In a fourth aspect, the injection system according to the first aspect, features a guide tube that comprises a telescoping two-part trombone slide mechanism comprising: (x) an outer cylindrical cannula comprising a first lumen and (y) an inner cannula; wherein the inner cannula has a distal and proximal end, further wherein the proximal end of the inner cannula is dimensioned to slide snugly within the lumen of the outer cannula, and further wherein the distal end of the inner cannula is bent relative to the proximal end of the inner cannula.

In a fifth aspect, the delivery catheter is secured to the lumen of the second cannula at a location proximal to the path of the inner cannula within the lumen of the outer cannula, and further wherein the second cannula is connected to the first linear actuator.

In a sixth aspect, the injection system has a second linear actuator, wherein the first linear actuator is configured to extend and retract the delivery catheter through the guide tube and the second linear actuator is configured to actuate the plunger of the syringe.

In a seventh aspect, the injection system further comprises a programmable controller capable of controlling (a) the first linear actuator to advance and retract the delivery catheter, and (b) to control the second linear actuator to depress the plunger rod, thereby controlling the volume and flow rate of the liquid composition from the syringe.

In an eighth aspect, the delivery catheter forms a service loop at the proximal end between the first linear actuator and the syringe, thereby preventing kinking of the proximal end of the delivery catheter when the first linear actuator actuated.

In a ninth aspect and a preferred embodiment of the invention, the injection system injection further comprises a stereotaxic assembly comprises a goniometer comprising a macro-angular adjustment and/or a micro-angular adjustment for defining the angle of entry of the delivery catheter in the x, y and z axes relative to the axis of the spinal cord of the subject positioned adjacent to the delivery catheter. The goniometer may define different angles of entry of the delivery catheter, for example at an angle of ±90° relative to the axis of the spinal cord of the subject, at an angle of ±30° relative to the axis of the spinal cord of the subject, or ±15° relative to the axis of the spinal cord of the subject.

In additional aspects of the invention, the distal end of the delivery catheter is shaped in a needle point.

In further aspects of the invention, the injection system further comprises a vertical height adjustable post. See, for example, feature 904 of FIG. 18.

In still further aspects of the invention, the injection system further comprises an adjustable articulated arm. See, for example, feature 910 of FIG. 18.

In a preferred embodiment of the invention, the micro-positioning adjustment further comprises: a first horizontal support arm; a second horizontal support arm oriented at right angles to the first horizontal support arm; and a rotatable stage member; wherein the first horizontal support arm comprises one or more adjustable vertical support rail attached to a first vertical support rail micro-adjustor for adjusting the first horizontal support arm along the z axis; further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more rails attached at the top of the goniometer rail to the bottom surface of the rotatable stage.

In still further aspects of the invention, the outer cannula is attached to a first mounting block that connects to the first linear actuator.

In other embodiments of the present invention, the delivery catheter comprises a synthetic polymeric catheter, which, for example may be polyethylene or another medically acceptable polymeric material, including copolymers known in the art that are useful as catheters. Such catheters may have various configurations at the distal end, for example, blunt, pointed and tapered ends.

In alternative embodiments, the delivery catheter may comprise an elongated tube made of a shape memory and/or superelastic alloy, for example, nitinol.

In further embodiments of the present invention, the position of the distal end of the guide tube and, hence, the distal end of the delivery catheter may form a 90 degree angle or an obtuse angle of 91 to 180 degrees relative to the alignment of the spinal cord of the subject,

In yet further embodiments of the present invention, the anatomical space comprises a brain, a spinal cord, a subarachnoid space, a subpial space, a dura matter or a dural lining of the spinal cord, an intrathecal space, a pericardial space, a pleura, a seurosa, an intra-pleural space, a kidney, a renal capsule, a blood vessel or a blood vessel wall, a peritoneal cavity, an intra-abdominal space, an intrathoracic space, or any space in the body bounded by a membrane or membranous entity.

In still further embodiments of the invention, the medium comprises a pharmaceutically active substance, therapeutic cells, fluids, biological fluids, drugs, gene therapy vectors, irrigation fluids, nucleic acids, growth factors, nuclear medicine agents, antibiotics, anti-viral agents, contrast agents, chemotherapies, or other diagnostic substances or therapeutic substances.

In various alternative embodiments of the present invention, the therapeutic cells are selected from the group consisting of: neural stem cells, pre-differentiated cells in the neuronal lineage, glial cells, glial restricted progenitor cells, Schwann cells, olfactory ensheathing cells, fibroblasts, mesenchymal stem cells, adipose derived stem cells, induced pluripotent stem cells, embryonic stem cells, bone marrow derived stem cells, hematopoietic stem cells, genetically modified cells, and the differentiated progeny of any of the above. Neural stem cells may be differentiated or undifferentiated progeny of human neural stem cells.

In yet other embodiments of the present invention the pharmaceutically active substance is selected from the group consisting of Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, protein kinase inhibitors, small interfering RNAs, analogs, derivatives, and modifications thereof, and combinations thereof or other therapeutic agents.

In embodiments of the present invention where gene therapy is desired, a gene therapy vector comprising one or more viral vectors, nucleic acids, polymeric transfection agents may be employed.

In preferred alternative embodiments of the present invention, the anatomical space is a brain, spinal column, subarachnoid space, subpial space or injection below the dura matter or a dural lining of the spinal cord.

In still further embodiments, the anatomical space comprises an intrathecal space, a pericardial space, a pleura, a seurosa, an intra-pleural space, a kidney, a renal capsule, a blood vessel or a blood vessel wall, a peritoneal cavity, an intra-abdominal space, an intrathoracic space, or any space in the body bounded by a membrane or membranous entity.

In other embodiments of the present invention, the medium comprises a pharmaceutically active substance, therapeutic cells, fluids, biological fluids, drugs, gene therapy vectors, irrigation fluids, growth factors, nuclear medicine agents, antibiotics, anti-viral agents, contrast agents, chemotherapies, or other diagnostic or therapeutic substances.

In some embodiments of the present invention, the injection system further comprises further comprising a syringe pump for pumping the liquid medium comprising therapeutic cells and/or one or more therapeutic substance from the syringe to the flexible delivery catheter.

In a tenth aspect of the invention, the injection system may be used in a method for delivering a trail of therapeutic cells and/or one or more therapeutic substance or diagnostic substance or other injectable medium into an anatomical space of an animal or human subject, the method comprising: introducing the distal end of the delivery catheter into the anatomical space of a subject through the distal end of the guide tube of the injection system according to the first aspect; advancing the delivery catheter through actuation of the linear actuator along a trail inside the anatomical space; and retracting the delivery catheter along the trail by reversing the action of the linear actuator while delivering an injectable medium of therapeutic cells and/or one or more therapeutic substance or diagnostic substance or other injectable medium through the delivery catheter along the trail.

In eleventh and a preferred aspect, the injection system may be used in a method for delivering a trail of therapeutic cells and/or one or more therapeutic substance or diagnostic substance or other injectable medium into an anatomical space of an animal or human subject, the method comprising: introducing the distal end of the delivery catheter into the anatomical space of a subject through the distal end of the guide tube of the injection system according to the ninth aspect; advancing the delivery catheter through actuation of the linear actuator along a trail inside the anatomical space; and retracting the delivery catheter along the trail by reversing the action of the linear actuator while delivering an injectable medium of therapeutic cells and/or one or more therapeutic substance or diagnostic substance or other injectable medium through the delivery catheter along the trail.

In further embodiments the methods of the tenth and eleventh aspects may comprise at least one therapeutic substance which is selected from the group consisting of Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other therapeutic agents.

In a twelfth aspect of the present invention, the delivery of the trail of therapeutic cells and/or one or more therapeutic substance or diagnostic substance or other injectable medium according to the tenth aspect is imaged using magnetic resonance imaging, computed tomography, fluoroscopy, ultrasound, or other radiological modalities.

In a thirteenth aspect of the present invention, the delivery of the trail of therapeutic cells and/or one or more therapeutic substance or diagnostic substance or other injectable medium according to the eleventh aspect is imaged using magnetic resonance imaging, computed tomography, fluoroscopy, ultrasound, or other radiological modalities. In other embodiments the delivery of the trail of therapeutic cells and/or one or more therapeutic substance or diagnostic substance or other injectable medium is to a brain or a spinal cord.

In a fourteenth aspect of the invention, a method of treating an injury or disease of an anatomical space of an animal or human subject, comprising the step of delivery a trail of therapeutic cells and/or one or more therapeutic substance, or diagnostic substance, or other injectable medium into the anatomical space of a subject according to the method of the tenth aspect.

In a fifteenth aspect of the invention, a method of treating an injury or disease of an anatomical space of an animal or human subject, comprising the step of delivery a trail of therapeutic cells and/or one or more therapeutic substance, or diagnostic substance, or other injectable medium into the anatomical space of a subject according to the method of the eleventh aspect.

In a sixteenth aspect of the invention, a method of defining the delivery of the trail of therapeutic cells and/or one or more therapeutic substances or diagnostic substances or injectable medium into an anatomical space of an animal or human subject is described according to the preceding aspects of the invention, the method comprising: (i) obtaining a magnetic resonance image of the anatomical space; (ii) defining the angle of entry and length of the trail to be delivered; and (iii) applying the angle of entry and length of the trail to be delivered to the surgical approach by aligning the angles with intraoperative fluoroscopy or computed tomography markers.

With reference to delivery of an injectable medium to an anatomical space of an animal or human subject, a further embodiment of the injection system comprises: a) one or more linear actuator; b) an injector device sub-assembly for actuating (1) a separately provided injection needle subassembly and a (2) a separately provided pre-filled syringe In preferred embodiments, the administration and delivery of a trail of therapeutic cells and/or one or more therapeutic substances or diagnostic substances or injectable medium is to the brain or spinal cord; wherein the syringe comprises a needle connector at one end and a plunger attached to a plunger rod at the opposite end; wherein the delivery catheter/injection needle subassembly comprises a first telescoping guide tube having an inner cannula and an outer diameter; and a second cannula having a second inner cannula slidably engaged with the outer diameter of the first telescoping guide needle; a delivery catheter/injection needle inserted through the first inner and second inner cannulas and connecting at one end with the pre-filled syringe needle connector and optionally formed into a needle point at the opposite end; wherein the delivery catheter is secured to the interior surface of the second cannula; and wherein the second cannula and the plunger rod are connected to a linear actuator; c) a macro-positioning subassembly for orienting the delivery catheter in the x, y and z axes relative to a prone animal or human positioned adjacent the injection system; and d) a programmable controller capable of controlling the linear actuator to (i) advance and retract the delivery catheter/injection needle and (ii) to control the volume and flow rate of the injectable medium of the pre-filled syringe through actuation of the plunger rod in the operation of the injection system. In preferred embodiments, the administration and delivery is a trail of therapeutic cells and/or one or more therapeutic substances or diagnostic substances or injectable medium is to the brain or spinal cord.

In an alternative embodiment of the present invention for delivery of an injectable medium to an anatomical space of an animal or human subject, the embodiment comprises an injection system for delivering a trail of therapeutic cells and/or one or more therapeutic substances or diagnostic substances or injectable medium, comprising: a) a first linear actuator and a second linear actuator; b) an injector device sub-assembly for actuating (1) a separately provided delivery catheter/injection needle subassembly and a (2) a separately provided pre- filled syringe containing therapeutic cells and/or one or more therapeutic substances or diagnostic substances or injectable medium; wherein the syringe comprises a needle connector at one end and a plunger attached to a plunger rod at the opposite end; wherein the delivery catheter/injection needle subassembly comprises a first telescoping guide tube having an inner cannula and an outer diameter; and a second rigid cannula having a second inner cannula slidably engaged with the outer diameter of the first telescoping guide tube; a delivery catheter/injection needle inserted through the first inner and second inner cannulas and connecting at one end with the pre-filled syringe needle connector and formed into a needle point at the opposite end; wherein the delivery catheter is secured to the interior surface of the second rigid cannula; and wherein the second rigid cannula is connected to a first linear actuator and the plunger rod is connected to a second linear actuator; c) a macro-positioning subassembly for orienting the delivery catheter in the x, y and z axes relative to a prone animal or human positioned adjacent the injection system; and d) a programmable controller capable of controlling the linear actuators to (i) advance and retract the delivery catheter/injection needle and (ii) to control the volume and flow rate of the contents of the pre-filled syringe through actuation of the plunger rod in the operation of the injection system. In preferred embodiments, the administration and delivery of a trail of therapeutic cells and/or one or more therapeutic substances or diagnostic substances or injectable medium is to the brain or spinal cord.

In a preferred embodiment, a first linear actuator controls the telescoping outer cannula 945a \ injection needle 943. A second linear actuator actuates the syringe plunger 941c. The macro-positioning subassembly is configured to bring the distal end of the guide tube/needle 942 into proper position in use of the injection system.

In another embodiment either of the foregoing two embodiments of the invention may further comprise a goniometer comprising a macro-angular adjustment and/or a micro-angular adjustment, more completely described in Embodiment 2, set forth in the specification. The goniometer permits accurate pitch control of the guide tube/needle and enclosed delivery catheter/injection needle, thereby permitting injections according to varying geometries relative to the orientation of the anatomical space, in particular, the spinal cord parenchyma, of the subj ect.

In another embodiment of the invention according to any of the previous three embodiments of the invention, the injection system may further comprise a vertical height adjustable post, an adjustable articulated arm, as more completely described in Embodiment 4, set forth in the specification. The vertical height adjustable post and adjustable articulated arm permit precise positioning of the injector device subassembly to be oriented along the x, y and z axes r relative to the orientation of the spinal cord parenchyma of the subject.

In another embodiment of the invention any one of the foregoing four embodiments of the present invention, the injection system may further comprise a micro-angular adjustment subassembly; wherein the micro-angular positioning subassembly further comprises: a first horizontal support arm; a second horizontal support arm oriented at right angles to the first horizontal support arm; and a rotatable stage member; wherein the first horizontal support arm comprises one or more adjustable vertical support rail attached to a first vertical support rail micro-adjustor for adjusting the first horizontal support arm along the z axis; further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more second goniometer rail attached at the top of the goniometer rail to the bottom surface of the rotatable stage. Reference may be made to FIGS. 29A and 29B for such an embodiment. This alterative embodiment is further described in Embodiment 5 of the present invention, as set forth in the specification. Such embodiments are also discussed with reference to the specification and figures as an XYZ mounting system. The XYZ mounting system, as disclosed in the specification, provides very precise positioning of the guide tube/needle and delivery catheter/injection needle relative to the orientation of the spinal cord parenchyma of the subject. This is particularly true in a preferred embodiment when a goniometer pitch adjustment is utilized.

In still another embodiment of the present invention, an injection system for delivering an injectable medium to an anatomical space of an animal or human subject, particularly trail of therapeutic cells and/or one or more therapeutic substances or diagnostic substances or injectable medium into an anatomical space of an animal or human subject, the injection system comprises: a) at least one linear actuator; b) an injector device sub-assembly for actuating (1) a separately provided delivery catheter/injection needle subassembly and a (2) a separately provided pre- filled syringe containing therapeutic cells and/or one or more therapeutic substances or diagnostic substances or injectable medium; wherein the syringe comprises a needle connector at one end and a plunger attached to a plunger rod at the opposite end; wherein the delivery catheter/injection needle subassembly comprises (i) a flexible metallic catheter comprising a syringe needle connector capable of attaching to the needle connector of the pre-filled syringe at one end and, optionally, having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end (i.e. the distal end) into a guide needle; wherein the delivery catheter/injection needle is dimensioned to pass through the telescoping two-part slide mechanism; further wherein the delivery catheter is secured to the interior of the outer cannula thereby providing for vertical movement of the outer cannula and attached delivery catheter upon actuation of the linear actuator; and further wherein the delivery catheter is capable of forming a delivery catheter/injection needle service loop at the end of the catheter attached to the prefilled syringe; further wherein the outer cannula is attached to a first mounting block that connects to the first linear actuator connector between the injection needle subassembly and the linear actuator; and wherein the inner cannula is attached to a second mounting block that rigidly connects to the injection needle subassembly connector of the injector device subassembly; and c) a micro-positioning subassembly for orienting the flexible wire catheter in the x, y and z axes relative to a prone animal or human positioned adjacent the automated injection system; further comprising a vertical height adjustable post, an adjustable articulated arm; and d) a programmable controller capable of controlling the at least one linear actuator to (i) advance and retract the delivery catheter/injection needle and (ii) to control the volume and flow rate of the contents of the pre-filled syringe through actuation of the plunger rod in the operation of the automated injection system.

In a further embodiment of the present invention, the injection system comprises an injection system for delivery of an injectable medium to an anatomical space of an animal or human subject, particularly a trail of therapeutic cells and/or one or more therapeutic substances or diagnostic substances or injectable medium into an anatomical space of an animal or human subject, comprising a) a first and a second linear actuator; b) an injector device sub-assembly for actuating (1) a separately provided delivery catheter/injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by a second linear actuator; wherein the delivery catheter/injection needle subassembly comprises (i) a delivery catheter comprising a syringe needle connector capable of attaching to the needle connector of the pre-filled syringe at one end and, optionally, having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide tube/needle; wherein the flexible metallic needle is dimensioned to pass through the telescoping two-part slide mechanism; further wherein the delivery catheter is secured to the interior of the outer cannula thereby providing for vertical movement of the outer cannula and attached delivery catheter upon actuation of the first linear actuator; and further wherein the delivery catheter is capable of forming a delivery catheter/injection needle service loop at the proximal end of the catheter attached to the prefilled syringe; further wherein the outer cannula is attached to a first mounting block that connects to the first linear actuator connector between the delivery catheter/injection needle subassembly and the linear actuator; and wherein the inner cannula is attached to a second mounting block that rigidly connects to the delivery catheter/injection needle subassembly connector of the injector device subassembly; and c) a micro-positioning subassembly for orienting the delivery catheter in the x, y and z axes relative to a prone animal or human positioned under the automated injection device; further comprising a vertical height adjustable post, an adjustable articulated arm; and d) a programmable controller capable of controlling the at least one linear actuator to (i) advance and retract the delivery catheter/injection needle and (ii) to control the volume and flow rate of the contents of the pre- filled syringe through actuation of the plunger rod in the operation of the automated injection system.

In still another embodiment of the present invention, an injection system for delivering an injectable medium to an anatomical space of an animal or human subject, particularly trail of therapeutic cells and/or one or more therapeutic substances or diagnostic substances or injectable medium into an anatomical space of an animal or human subject, the injection system comprises: a) a first and a second linear actuator; b) an injector device sub-assembly for actuating (1) a separately provided injection needle subassembly and a (2) a separately provided pre-filled syringe comprising therapeutic cells and/or one or more therapeutic substances or diagnostic substances or injectable medium; wherein the syringe comprises a needle connector at one end and a plunger attached to a plunger rod at the opposite end; wherein the plunger rod is actuated by the second linear actuator; wherein the injection needle subassembly comprises (i) a delivery catheter comprising a syringe needle connector capable of attaching to the needle connector of the pre-filled syringe at one end and, optionally, having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide tube/needle; wherein the delivery catheter needle is dimensioned to pass through the telescoping two-part slide mechanism; further wherein the delivery catheter is secured to the interior of the outer cannula thereby providing for vertical movement of the outer cannula and attached delivery catheter upon actuation of the first linear actuator; and further wherein the delivery catheter is capable of forming a delivery catheter/injection needle service loop at the end of the catheter attached to the prefilled syringe; further wherein the outer cannula is attached to a first mounting block that connects to the first linear actuator connector between the injection needle subassembly and the linear actuator; and wherein the inner cannula is attached to a second mounting block that rigidly connects to the injection needle subassembly connector of the injector device subassembly; and c) a micro-positioning subassembly for orienting the delivery catheter in the x, y and z axes relative to an animal or human adjacent the injection system; further comprising a goniometer comprising a macro-angular adjustment; a vertical height adjustable post, an adjustable articulated arm; and d) a programmable controller capable of controlling the first and second linear actuator to (i) advance and retract the injection needle and (ii) to control the volume and flow rate of the contents of the pre-filled syringe through actuation of the plunger rod in the operation of the automated injection device.

In further embodiment of the present invention, an injection system for delivering an injectable medium to an anatomical space of an animal or human subject, particularly a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord; a first guide tube/introducer needle having a proximal end and a distal end, wherein: the first guide tube houses a portion of the distal end of the delivery catheter, and the first guide tube is configured to introduce the distal end of the delivery catheter into the spinal cord; a linear actuator located near the proximal end of the first guide tube/introducer needle and configured to move the delivery needle inside the first guide tube/introducer needle; and a second guide tube located between the linear actuator and the proximal end of the delivery catheter/introducer needle, wherein the second guide tube houses and guides a portion of the delivery needle between the linear actuator and the proximal end of the delivery catheter.

In yet another embodiment of the invention, an injection system is provided for delivering an injectable medium to an anatomical space of an animal or human subject, particularly a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) a first and a second linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by the second linear actuator; wherein the injection needle subassembly comprises (i) a delivery catheter comprising a syringe needle connector capable of attaching to the needle connector of the pre-filled syringe at one end and having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide tube/needle; wherein the delivery catheter is dimensioned to pass through the telescoping two-part slide mechanism; further wherein the delivery catheter is secured to the interior of the outer cannula thereby providing for vertical movement of the outer cannula and attached delivery catheter upon actuation of the first linear actuator; and further wherein the delivery catheter is capable of forming a delivery catheter/injection needle service loop at the end of the catheter attached to the prefilled syringe; further wherein the outer cannula is attached to a first mounting block that connects to the first linear actuator connector between the injection needle subassembly and the first linear actuator; and wherein the inner cannula is attached to a second mounting block that connects to the injection needle subassembly connector of the injector device subassembly; and c) a macro-positioning subassembly for orienting the flexible wire catheter in the x, y and z axes relative to an animal or human positioned adjacent the automated injection device; further comprising a goniometer comprising a macro-angular adjustment; a vertical height adjustable post, an adjustable articulated arm; further comprising a vertical height adjustable post, an adjustable articulated arm and a micro-positioning subassembly; wherein the micro-positioning subassembly further comprises: a first horizontal support arm; a second horizontal support arm oriented at right angles to the first horizontal support arm; and a rotatable stage member; wherein the first horizontal support arm comprises one or more adjustable vertical support rail attached to a first vertical support rail micro-adjustor for adjusting the first horizontal support arm along the z axis; and further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more rails attached at the top of the goniometer rail to the bottom surface of the rotatable stage; and d) a programmable controller capable of controlling the linear actuator to (i) advance and retract the delivery catheter/injection needle and (ii) to control the volume and flow rate of the contents of the pre-filled syringe through actuation of the plunger rod in the operation of the automated injection device.

In yet another embodiment of the present invention, an injection system is provided for delivering an injectable medium to an anatomical space of an animal or human subject, particularly a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) an injector device subassembly comprising: (1) an injection needle subassembly; (2) a separately provided prefilled syringe comprising an injection needle connector at one end and a plunger connected to a plunger rod; (3) a linear actuator; (4) one or more injector device subassembly mounting connectors; (5) an injection needle subassembly connector; (6) a first linear actuator connector between the injection needle subassembly and the linear actuator; and (7) a second linear actuator connector between the plunger rod and the linear actuator, wherein the second linear actuator connector is capable of controlling the volume and flow rate of the pre-filled syringe by actuation of the plunger rod in the operation of the injection system; b) a macro-positioning sub-assembly for roughly adjusting the orientation of the automated injector device sub-assembly along x, y and z axes relative to an animal or human positioned adjacent the automated injection device, comprising a vertical height adjustable post, an adjustable articulated arm, and a micro-positioning subassembly; wherein the micro-positioning subassembly further comprises: a first horizontal support arm; a second horizontal support arm oriented at right angles to the first horizontal support arm; a rotatable stage member; and a goniometer comprising goniometer a macro-angular adjustment and a goniometer micro-angular adjustment; wherein the first horizontal support arm comprises one or more adjustable vertical support rail attached to a first vertical support rail micro-adjustor for adjusting the first horizontal support arm along the z axis; and further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more rails attached at the top of the goniometer to the bottom surface of the rotatable stage; c) further comprising a separately provided injection needle subassembly, wherein the injection needle subassembly comprises: (i) a delivery catheter comprising a syringe needle connector capable of attaching to the needle connector of the pre-filled syringe at one end and having a needle point at the other end of the delivery catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide tube/needle; wherein the delivery catheter is dimensioned to pass through the telescoping two-part slide mechanism; further wherein the delivery catheter is secured to the interior of the outer cannula thereby providing for vertical movement of the outer cannula and attached delivery catheter upon actuation of the linear actuator; and further wherein the delivery catheter is capable of forming a delivery catheter/injection needle service loop at the end of the catheter attached to the prefilled syringe; further wherein the outer cannula is attached to a first mounting block that connects to the first linear actuator connector between the injection needle subassembly and the linear actuator; and wherein the inner cannula is attached to a second mounting block that rigidly connects to the injection needle subassembly connector of the injector device subassembly; and d) a programmable controller capable of controlling volume and flow rate of the pre-filled syringe in operation.

In a further embodiment of the present invention, a method of injecting an injectable medium to an anatomical space of an animal or human subject, particularly a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the central nervous system, in particular, directly into the spinal cord parenchyma, employing the injection apparatus of any one of the foregoing aspects of the present invention.

In another aspect of the present invention, a method for delivering an injectable medium to an anatomical space of an animal or human subject, particularly a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord is described, the method comprising: positioning a distal end of an guide tube/introducer needle at a location near a target point in the spinal cord, wherein the guide tube/introducer needle houses a delivery catheter; introducing the delivery catheter into the spinal cord through the distal end of the guide tube/introducer needle; advancing the delivery catheter along a trail inside the spinal cord; and retracting the delivery catheter along the trail while delivering a liquid through the delivery catheter along the trail. It will be appreciated by the skilled artisan that according to certain embodiments of the invention, the guide tube may be rotated 180° to facilitate injecting a second a trail of therapeutic cells and/or one or more therapeutic substances or diagnostic substances or injectable medium that meets at the apex of the first trail, thereby forming a “tent-like” configuration

### BRIEF DESCRIPTION OF THE DRAWINGS

The patent or application file contains at least one drawing executed in color. Copies of this patent or patent application publication with color drawings) will be provided by the Office upon request and payment of the necessary fee.

The accompanying drawings, which are incorporated in this specification and constitute a part of it, illustrate several embodiments consistent with the disclosure. Together with the description, the drawings serve to explain the principles of the disclosure. In certain instances, the drawings may not necessarily be drawn to scale or be exhaustive; instead, emphasis is generally placed upon illustrating the principles of the embodiments described herein. A more complete understanding of the present invention, and the advantages and features of the present invention, will be readily understood by reference to the following detailed description when considered in conjunction with the accompanying drawings, wherein:

FIG. 1A illustrates treatment of a spinal cord injury using a perpendicular bolus injection method.

FIG. 1B illustrates treatment of a spinal cord injury using a longitudinal trail delivery method according to some embodiments.

FIG. 2 shows an image of a therapeutic trail delivery system according to some embodiments.

FIG. 3A is a front view image and FIG. 3B is a side view image of a stepper motor-based rotary friction drive linear actuation mechanism and parts related to controlled advancement and retraction of a delivery needle according to some embodiments.

FIG. 4 shows an image of a section of a therapeutic trail delivery system according to an embodiment.

FIG. 5 shows an image of a lower section of an injection system according to an embodiment.

FIG. 6A-6B show images of an introducer needle holder in an injection system according to some embodiments.

FIG. 7A-7E show images of guide tubes/introducer needles and delivery catheters/needles according to various embodiments.

FIGS. 8A-8D show sequential images of creating a cell trail in an experimental medium by a therapeutic trail injection system according to one embodiment.

FIG. 9 shows alignment of an injection needle above a porcine spinal cord according to an embodiment.

FIG. 10 shows a fluoroscopic image of a delivery needle within a porcine spinal cord according to an embodiment.

FIG. 11 shows a magnetic resonance image of a cell trail in a porcine spinal cord according to one embodiment

FIG. 12 illustrates cell suspension in various cell delivery media according to one embodiment.

FIG. 13 illustrates the delivery of neural stem cells into a spinal cord mimetic gel according to an embodiment.

FIG. 14 shows an in vitro trail of cells delivered into a spinal cord mimetic media according to one embodiment.

FIG. 15 shows a trail of cells delivered into a rat spinal cord according to one embodiment.

FIG. 16 shows images of rat neural stem cells in hyaluronic acid stored in a syringe for up to 40 h according to an embodiment.

FIG. 17 shows trypan blue staining of rat neural stem cells stored in a 0.75 wt. % hyaluronic acid carrier at 4° C. according to one embodiment.

FIG. 18 shows an image of a therapeutic trail injection system according to some embodiments of the present invention assembled on an optional mobile cart.

FIG. 19 is an image of an injection dispensing device apparatus comprising a syringe containing therapeutic cells and/or one or more therapeutic substances or diagnostic substances or injectable medium, a guide needle, an injection needle and an adjustable goniometer for pitch adjustment as well as a motorized injection needle assembly terminating in an injection needle.

FIG. 20 is an image of a disposable injection delivery catheter/needle assembly and prefilled syringe according to one embodiment.

FIG. 21A and FIG. 21B is a graphical representation of a mobile cart which optionally may be used to position the injection device next to a surgical bed or operating table, showing the support pedestals of the cart, wheels and locking mechanisms on the wheels to firmly position the mobile cart and injection device near the subject.

FIG. 22A and 22B are graphical representations of different views of a macro height adjustment mechanism affixed to mobile cart and macro height post 904 supporting a selective compliance articulated robot arm (SCARA positioning arm) and an injection dispensing device, in both use (FIG. 22A) and rest positions (FIG. 22B).

FIG. 23 is an enlarged graphical representation of a macro height adjustment mechanism for vertical extension of the macro height post.

FIG. 24 is an enlarged graphical representation of an embodiment of a selective compliance articulated robot arm (SCARA positioning arm) in use.

FIG. 25 is a graphical representation showing adjustment of the angle of guide needle by adjusting the angle by altering the angular positon on the goniometer.

FIG. 26 is a graphical representation showing a different adjustment of the angle of guide needle by adjusting the angle by altering the angular position of goniometer compared to FIG. 25.

FIG. 27 is a graphical representation of the operation and adjustment of the SCARA positioning arm to permit orientation of the injection dispensing device and guide needle along the “x” and “y” directions in use.

FIGS. 28A and 28B are enlarged graphical representations of SCARA arm adjustments and SCARA arm showing the adjustment of SCARA arm in relation to macro height adjustment post in FIG. 28A.

FIGS. 29A and 29B are enlarged graphical representations of micro adjustment mechanisms for an injection dispensing device to be mounted on a SCARA positioning arm (not shown).

FIG. 30 is a graphical representation of a complete trombone assembly joined to a delivery catheter/injection needle formed into a service loop at the proximal end and a curved guide tube/needle on the distal end and supported by snap-on connectors.

FIG. 31 is a graphical representation of an outer trombone tube and an inner trombone tube position in plastic snap on tabs.

FIG. 32A to 32D are graphical representations of joining the lower trombone tube to a snap-on tab (FIGS. 32A and 32B) and an outer trombone tube to a snap-on tab (FIGS. 32C and 32D).

FIG. 33 is a graphical representation of a partial assembly of the telescoping cannula 945A and 945B mounted to snap-on tabs connectors 946.

FIG. 34A, FIG. 34B, and FIG. 34C is a graphical representation of the injection angles to be used in Example 1.

FIG. 35 is a graphical representation of the surgical procedure set-up of experimental trail injection system 900 as it will be employed in Example 1.

FIG. 36 is an illustration of a programmable controller of an embodiment of the present invention.

FIG. 37 is a graphical representation of an injection device positioned on a mobile cart and attached to an operating table or a surgical bed.

FIG. 38A and FIG. 38B are graphical representations of a monopod support for an embodiment of an injection device attached operating table or a surgical be and tensioned to the floor.

FIG. 39 is a graphical representation of a bridge bed rail for support of an injection device.

FIG. 40 is a graphical representation of a cart bridge support for an injection device over a human subject positioned prone on an operating table or surgical bed.

FIG. 41 is a graphical representation of a selective compliance articulated robot arm (SCARA) positioning arm of an embodiment.

FIGS. 42A and 42B are graphical representations of the positioning of the SCARA positioning arm and injection device in use positioned over a human subject in the prone position on an operating table or surgical bed.

FIG. 43 is an illustration of a dual SCARA arm support for an injection device positioned over an animal subject.

FIG. 44 is a graphical representation of an XYZ mounting system for positioning the injection device above a surgical bed or operating table

FIG. 45 is a graphical representation of an injection device mounted on a mobile cart for positioning the injection device above a surgical bed or operating table comprising an embodiment of an XYZ mounting system.

FIG. 46 is a graphical representation of an embodiment of the telescoping cannula/trombone mechanism and the motorized syringe mechanism for actuating the plunger rod and injection needle.

FIGS. 47A and 47B are graphical representations of an embodiment of the injection needle subassembly illustrating the mechanized actuation of the syringe plunger rod and injection needle through the guide or introducer needle.

FIG. 48 is a graphical representation of a disposable telescoping guide tube/trombone assembly.

FIG. 49 is a graphical representation of an embodiment of a remote center angle adjustment positioning apparatus.

FIGS. 50A, 50B, and 50C are graphical representations of an embodiment of an adjustable goniometer for controlling pitch of the guide needle and injection needle.

FIGS. 51A and 51B are photographs of methylene blue trails injected at an angle in a “tent” formation around a prophetic injection site.

FIG. 52A provides a graphic representation of and attachment block to which a telescoping cannula assembly (trombone assembly) may be attached, in one embodiment, by an epoxy adhesive.

FIG. 52B is a photographic showing lower trombone cannula 945b attached by an epoxy adhesive to attachment block 946.

FIG. 52C is a graphic representation of lower trombone cannula 945b showing a 100° bend angle.

FIG. 53 is a graphic representation of the angle measurements in accordance with the injection of a 20 mm trail of the liquid composition of HA and methylene blue in accordance with Example 3.

FIG. 54 depicts the testing setup for injection device 900 used in this Example 3.

FIGS. 55A and 55B are images of methylene blue trails from a liquid composition comprising HA and methylene blue injected into an agarose slab at a setting of 2 mm and an injection angle of 5.7° in accordance with Example 3. FIGS. 56A, 56B and 55C are images of methylene blue trails from a liquid composition comprising HA and methylene blue injected into an agarose slab at a setting of 4 mm at an injection angle of 11.5°, 6 mm at an injection angle of 17.5° and at 8 mm at an injection angle of 23.6° in accordance with Example 3.

FIG. 57 is an image of a guide needle positioned at the surface of an agarose gel slab and an injection needle penetrating the agarose gel slab at a setting of 8 mm and an injection angle of 23.6° yielding a trail of 8 mm in accordance with Example 3.

FIG. 58 contains data from Example 5 including actual and expected fluid rates, needle travel and total dispensed volume.

FIG. 59 is a graphic representation of an injector dispensing device 940 and in a preferred embodiment two linear actuators 959a and 959b.

FIG. 60 is a graphic representation of preferred embodiment of an injector dispensing device, as discussed in connection with FIG. 59 above, with a syringe 941 attached.

FIG. 61 is a graphic representation of an injector dispensing device 940 showing in more detail the positioning of linear actuators 959a and 959b in a preferred embodiment.

FIG. 62 is a graphic representation of a goniometer 950 used in a preferred embodiment of the injector dispensing device 940. A preferred embodiment of goniometer is shown depicting macro-angle adjustments 1600 and micro-angle adjustments 1601. It will be appreciated by the skilled worker that the macro-angle adjusters 1600 and micro-angle adjusters 1601 may be configured in a number of way to provide the same function as those depicted in FIG. 62.

FIGS. 63A and 63B are photographs depicting three trails of hyaluronic acid-methylene blue in agarose demonstrating consistent trail angles in FIG. 63B.

FIG. 64 shows human neural stem cells delivered in a trail into a nude rat spinal cord after one month. The cells were labeled for STEM121 and doublecortin (DCX) markers, showing cell survival and neuronal precursor differentiation.

FIG. 65 shows survival of STEM121 labeled human neural stem cells delivered in a trail through a contusion injury in a nude rat. This demonstrates that cell trails can bridge injuries in the spinal cord and survive.

FIG. 66 shows cross-sections and longitudinal sections of STEM121 labeled human neural stem cells after 1 week delivered in a trial in a porcine spinal cord.

FIG. 67 depicts MRI images depicting how MRI imaging can be used to guide the trajectory trails of therapeutic cells and/or one or more therapeutic substances or diagnostic substances or injectable medium into an anatomical space of an animal or human subject.

FIG. 68 shows a photograph of a disposable trombone assembly with snap-on connectors 946 housing a polymeric polyethylene tubing (PE-5 catheter) 943 extruding from the curved guide needle 945.

FIG. 69 shows a photograph of a trombone assembled fitted with polyethylene tubing (PE-8 catheter) secured with snap-on connectors to the linear actuator and fixed connector portion of the injection system. Methylene blue solution was loaded into the attached syringe and flowed through the PE-8 tubing.

FIGS. 70A and 70B shows photographs of a polyethylene catheter (PE-8) extruded through the guide tubing and into the subpial space of a rat for injection of therapeutics. FIG. 70A shows a photograph of the PE-8 catheter in the subpial space and FIG. 70B shows a photograph of methylene blue injected through the catheter into the subpial space.

### DETAILED DESCRIPTION OF THE INVENTION

The following detailed description refers to the accompanying drawings. The same or similar reference numbers may be used in the drawings or in the description to refer to the same or similar parts. Also, similarly named elements may perform similar functions and may be similarly designed, unless specified otherwise. Details are set forth to provide an understanding of the exemplary embodiments. Embodiments, e.g., alternative embodiments, may be practiced without some of these details. In other instances, well known techniques, procedures, and components have not been described in detail to avoid obscuring the described embodiments.

The creation of one or more continuous trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject may overcome the aforementioned limitations of multiple injections known in the art. Moreover, embodiments of the present invention do not require using an endoscope, thereby enhancing the accuracy in positioning the injection needle to inject a trail of cells and/or a therapeutic substance. Various embodiments enable creating a trail of cells and/or a therapeutic substance in the spinal cord. In various embodiments, the technique is safe, easy, or reproducible. With regard to administering a trail of therapeutic cells and/or one or more therapeutic substances or diagnostic substances or injectable medium into the spinal cord of an animal or human subject, the goal of safety may be accomplished by minimizing the manipulation of the spinal cord, limiting the number of injection sites, or limiting the size of needle puncture. More particularly, the present invention permits substantial control over the entry angle of the delivery catheter/injection needle so that injections may be made rostral to caudal and caudal to rostral of an injury site and, furthermore, at injection angles that enable creating a “tent” of injection trails around the injury site.

At each area where the spinal cord is punctured by a delivery catheter or needle (an “injection site”), some degree of injury may result. In order to treat a segment of spinal cord while minimizing injection site-associated secondary injury, it may be advantageous to distribute therapeutic substance within the segment through as few injection sites as possible (single injection site, two injection sites, etc.). In some embodiments, a trail of therapeutic cells and/or one or more therapeutic substances or diagnostic substances or injectable medium through a single injection site would minimize secondary injury. Also, an ease of use and reproducibility goals may be accomplished by stereotaxically positioning the trail, controlled and automated trail creation, or integrated visualization methodologies.

In some embodiments, the trail may be created in the subpial space of the spinal cord. In such embodiments, subpial delivery may reduce damage to the spinal cord compared to parenchymal injection and improve therapeutic delivery compared to intrathecal, epidural, or systemic therapeutic administration.

In some embodiments, a trail is created by first introducing a delivery catheter into the spinal cord with a controlled path and rate of entry at a single injection site. Then, a trail of the therapeutic cells and/or one or more therapeutic substances or diagnostic substances or injectable medium is deposited by a controlled retraction of the delivery needle coupled with ejection of the therapeutic substance through the needle. Some embodiments deliver a homogenous trail of therapeutic cells that may settle in aqueous solutions, such as cells, drug-loaded particles, or other solids. In such embodiments, the delivery media may include a shear-thinning polymer or viscous liquid, such as hyaluronic acid.

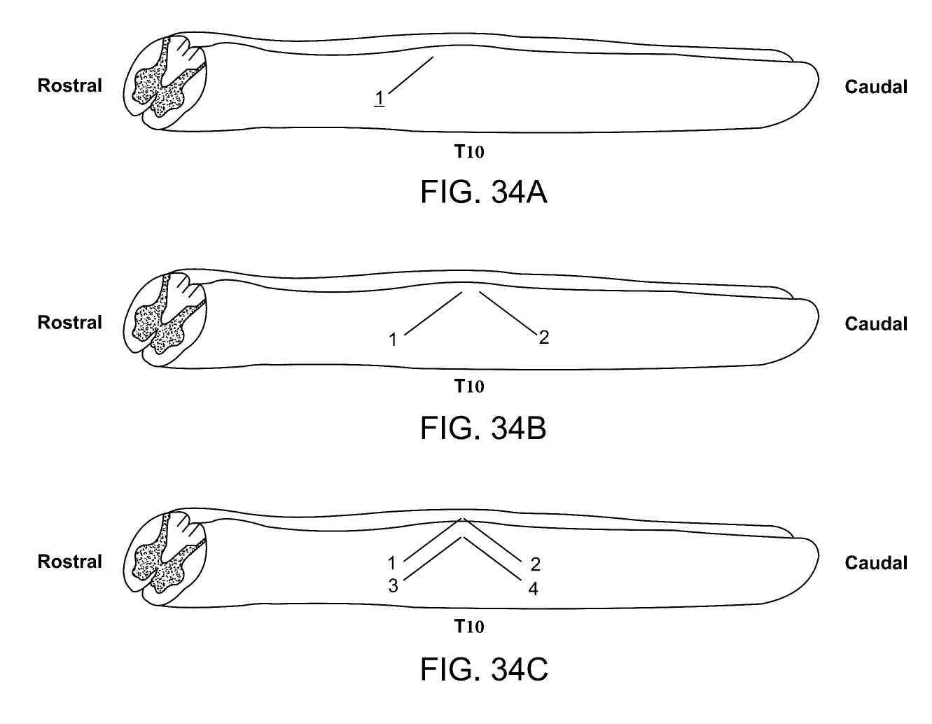

In order embodiments, multiple injections into the spinal cord parenchymal tissue in a rostral to caudal and/or caudal to rostral direction. In still other embodiments, a “tent” of injection trails may be deposited in the manner depicted in FIG. 34C and Example 1.

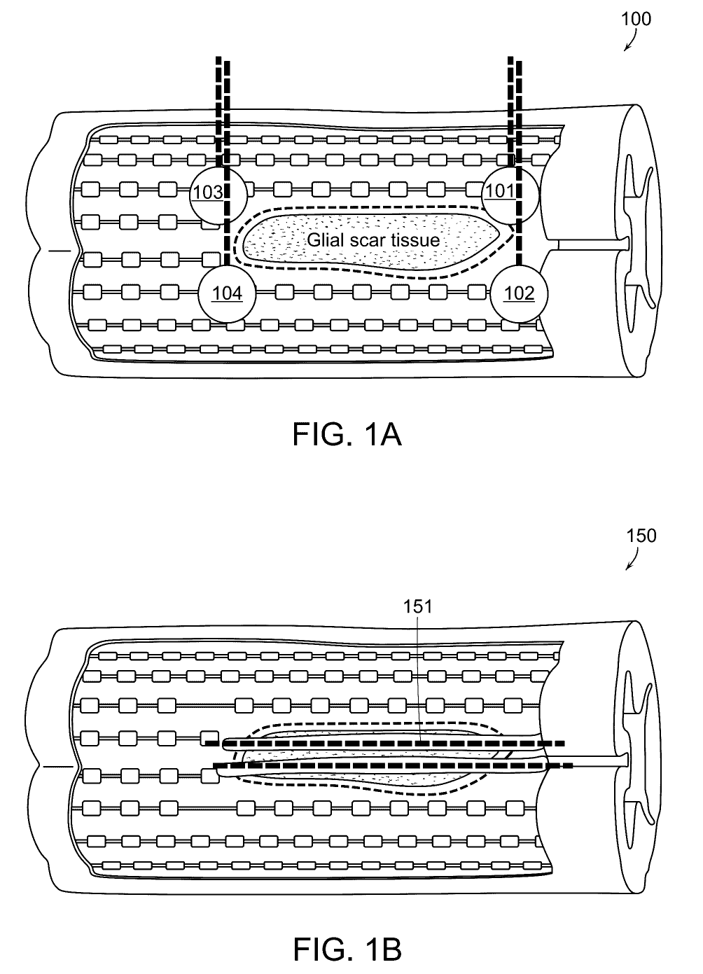

FIG. 1A is an illustration 100 of a perpendicular bolus injection method. In this method a needle is introduced perpendicular to the spinal cord and a defined volume of therapeutic substance is injected at each injection site. The needle remains in place during the injection and a spherical bolus is formed at the injection site.

Some delivery strategies may employ multiple bolus injections perpendicular to the surface of the spinal cord. FIG. 1, for example, shows four bolus injections at injection sites 101-104. Separated bolus injections, however, may not yield cell connectivity between the injection sites. In particular, the bolus injections may not connect, and communicate, with each other. Furthermore, in this method, the therapeutic substance may not be injected within injury site. The perpendicular bolus injection method, therefore, may fail to treat a damaged continuous segment of the spinal cord. Such failure may be particularly relevant for the treatment of the spinal cord injury where it is therapeutically important to rebuild neuronal connectivity across a spinal cord lesion or cystic cavity.

Moreover, the perpendicular bolus injection method may cause reflux. Reflux occurs when, during its delivery, the therapeutic substance travels in a direction that is the reverse of the injection direction, that is, up the needle track and out of the spinal cord during delivery. Due to reflux, the dose of the injected therapeutic substance may become inconsistent and may not match the anticipated dose.

FIG. 1B is an illustration 150 of a longitudinal therapeutic trail delivery method according to some embodiments. In such embodiments, one or more trails of a therapeutic substance (also called herein therapeutic trail may be deposited across an injury site (glial scar, in this depiction). Illustration 150 includes one such trail labeled 151. If the therapeutic substance includes cells, the trail may facilitate creating connectivity between cells within the trail or a bridge between the two damaged ends of spinal cord. Furthermore, the retraction of the delivery catheter during injection may reduce reflux and enable accurate therapeutic dosing.

The longitudinal cell trail delivery method results in a connected path of delivery for the delivered therapeutic substance. In this method, the therapeutic substance can be delivered with relatively higher dosage accuracy, reducing the risk of reflux. Moreover, it results in a higher surface area for the contact between the injured tissues and the therapeutic substance, thus increasing the chance and rate of recovery. In some embodiments, the therapeutic substance may be a suspension of therapeutic cells. The therapeutic cells may include, for example, neural stem cells, pre-differentiated cells in the neuronal lineage, glial cells, glial restricted progenitor cells, fibroblasts, mesenchymal stem cells, adipose derived stem cells, induced pluripotent stem cells, embryonic stem cells, or other cells types. Neural stem cells or pre-differentiated cells may connect with the neuronal circuitry on both sides of a spinal cord injury and form a bridge across the injury site. This connected bridge may serve to replace lost neuronal connection and return some impaired function.

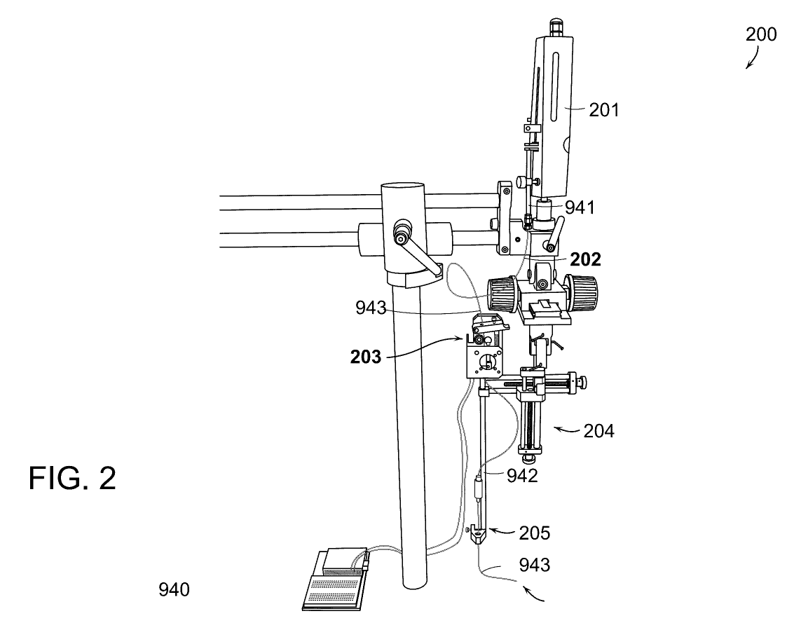

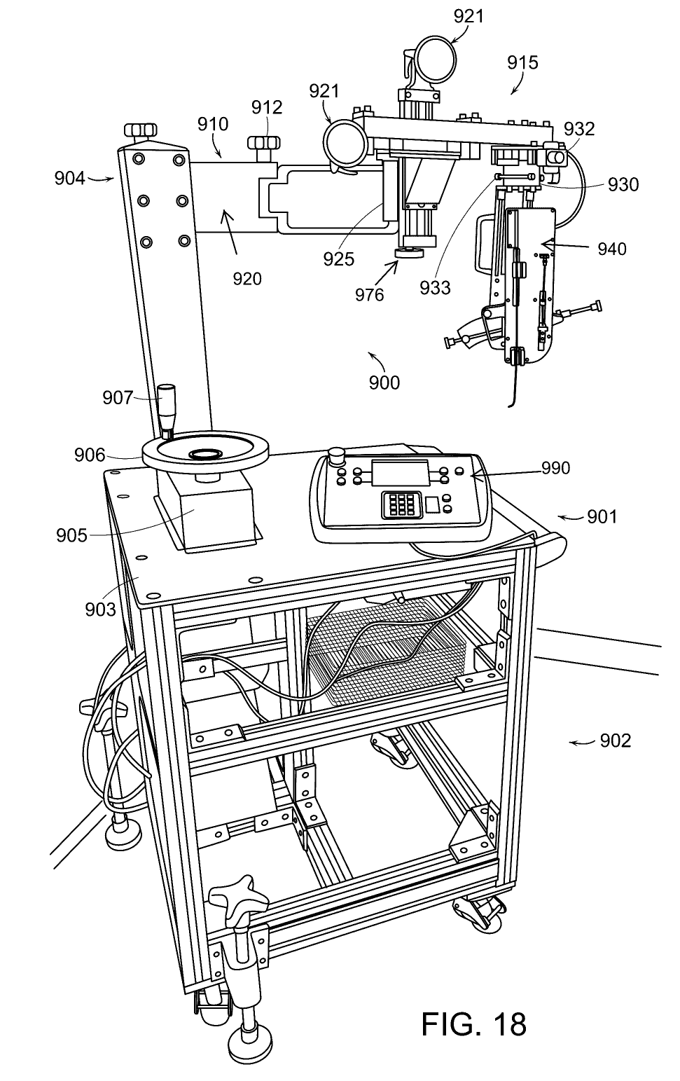

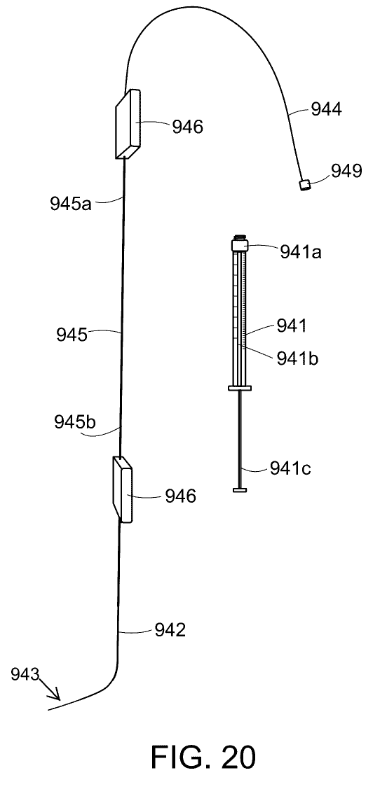

FIG. 2 shows an image of a cell trail delivery system 200 according to some embodiments. System 200 includes a syringe pump 201 a syringe 941 (see FIG. 20), a flexible tubing 202, a stepper motor-based rotary friction drive 203, a motor controller 990 (see FIG. 18), a stereotaxic apparatus 204, a combined guide tubing and introducer needle 942, an guide tube/introducer needle holder 205, and a delivery needle 943.

In system 200, the delivery needle is configured to enter the spinal cord by its distal end and deliver a therapeutic substance inside the spinal cord or in the subpial area, i.e. beneath the pia matter. The delivery needle may create a trail inside the spinal cord and deposit the therapeutic cells and/or one or more therapeutic substances or diagnostic substances or injectable medium on that trail. In some embodiments, the delivery catheter is made of a shape memory and/or superelastic material such as a shape memory alloy, e.g., nitinol (nickel-titanium alloy), and may alternatively be called nitinol needle.

The guide tube/introducer needle is configured to house the delivery catheter. The guide tube has a proximal end and a distal end. The guide tube may have a curved section near its distal end. The proximal end may be held by the introducer needle holder connected to the stereotaxic apparatus. A user, such a surgeon, may move the proximal end using the stereotaxic apparatus, thus being able to move the distal end in all directions. The user may thus place the distal end at a location near the injured site in the spinal cord. The delivery needle may then exit the introducer needle through its distal end and enters the spinal cord. The delivery needle may advance through the spinal cord to create the trail and deposit the trail of therapeutic cells and/or one or more therapeutic substances or diagnostic substances or injectable medium. In some embodiments, the delivery catheter straightens after exiting the distal end of the guide tube/introducer needle and thus creates a straight line trail.

The guide tubing is attached to the linear actuator on one end and the proximal end of the introducer needle on the other end. In an embodiment, the delivery needle enters the guide tubing after threading through the linear actuator and exits the guide tubing at its attachment with the delivery needle, to enter the delivery needle at its proximal end. The guide tubing guides the delivery needle between the linear actuator and the proximal end of the introducer needle. This guide tubing may be used in the linear actuator-driven advancement of the delivery catheter to prevent buckling of the delivery needle between the linear actuator and introducer needle.

The linear actuator moves the delivery needle back and forth, thus causing it to, for example, exit the distal end of the introducer needle, enter the spinal cord, create the trail, or retract while depositing the therapeutic cells and/or one or more therapeutic substances or diagnostic substances or injectable medium. The motor controller controls the operation of the linear actuator. The controller may move rotate the linear actuator in a forward or a backward direction to move the delivery needle forward or backward, respectively. The controller may be operated by a user or programed to advance and retract at a controlled rate and for a controlled distance.

The syringe may be connected to a proximal end of the delivery catheter through a length of flexible tubing 202. The syringe and syringe pump 201 may inject the therapeutic substance through the flexible tubing 202 and into the delivery needle 943. The timing and flow rate of the injection may be synchronized with a linear actuator or a rotary friction drive 203 to coordinate the deposition of the therapeutic cells and/or one or more therapeutic substances or diagnostic substances or injectable medium with the location or speed of the distal end of the delivery catheter inside the spinal cord. This coordination may be used to deposit a desired amount of the therapeutic cells and/or one or more therapeutic substances or diagnostic substances or injectable medium at different points of the trail. The coordination may, for example, result in a uniform deposition of the therapeutic cells and/or one or more therapeutic substances or diagnostic substances or injectable medium along the trail, resulting with an optimum therapeutic result. The coordination may also be utilized for depositing a non-uniform trail with areas of less or more volume of therapeutic cells and/or one or more therapeutic substances or diagnostic substances or injectable medium. In some embodiments, the trail may pass, for example, through a cystic cavity in the spinal cord where the rate of retraction of the delivery needle or the flow rate of therapeutic cells and/or one or more therapeutic substances or diagnostic substances or injectable medium may be adjusted to fill the cystic cavity with a therapeutic substance. The diameter of the trail may be controlled by increasing or decreasing the amount of therapeutic cells and/or one or more therapeutic substances or diagnostic substances or injectable medium delivered in a given area. This may be controlled by factors that include adjusting the flow rate of injected substance or the retraction rate of the delivery catheter.

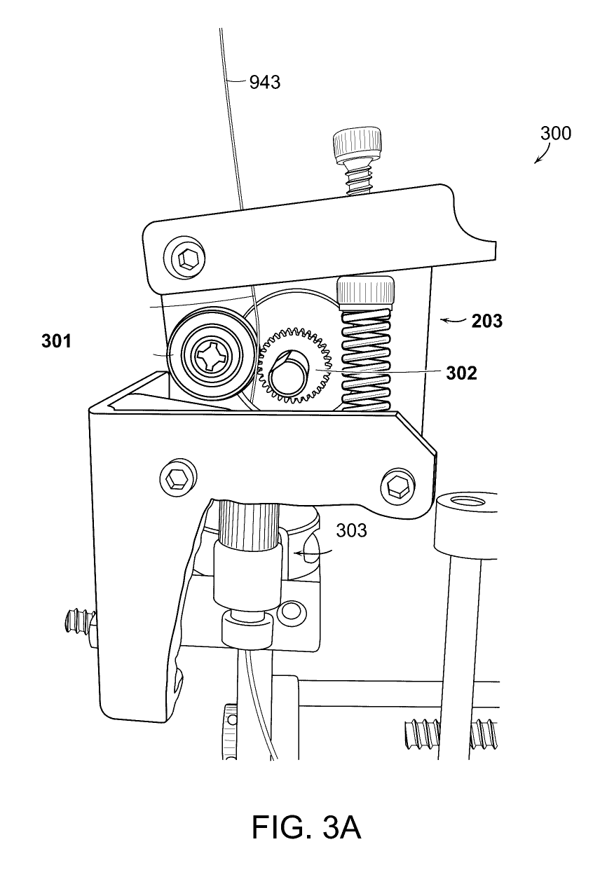

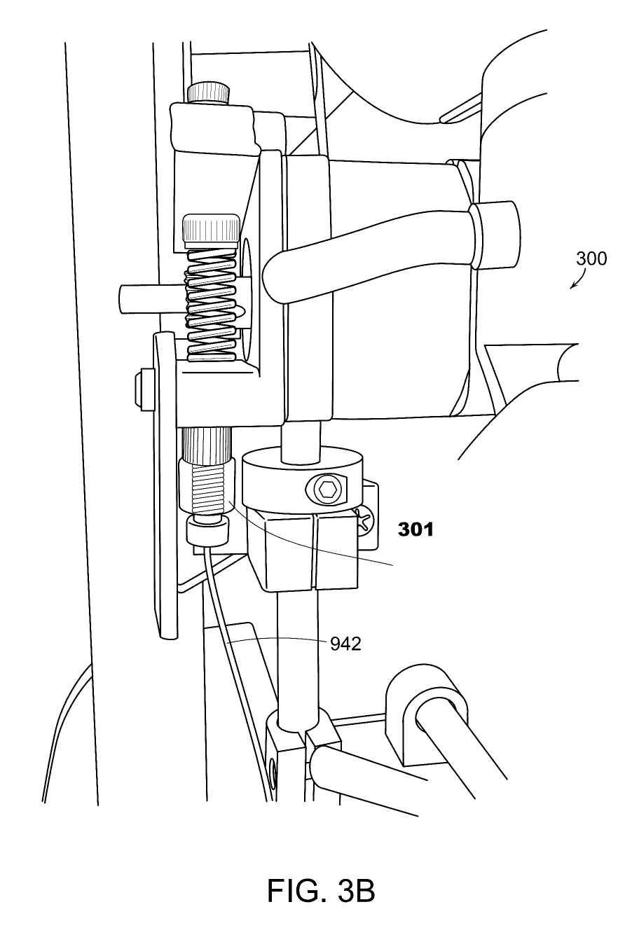

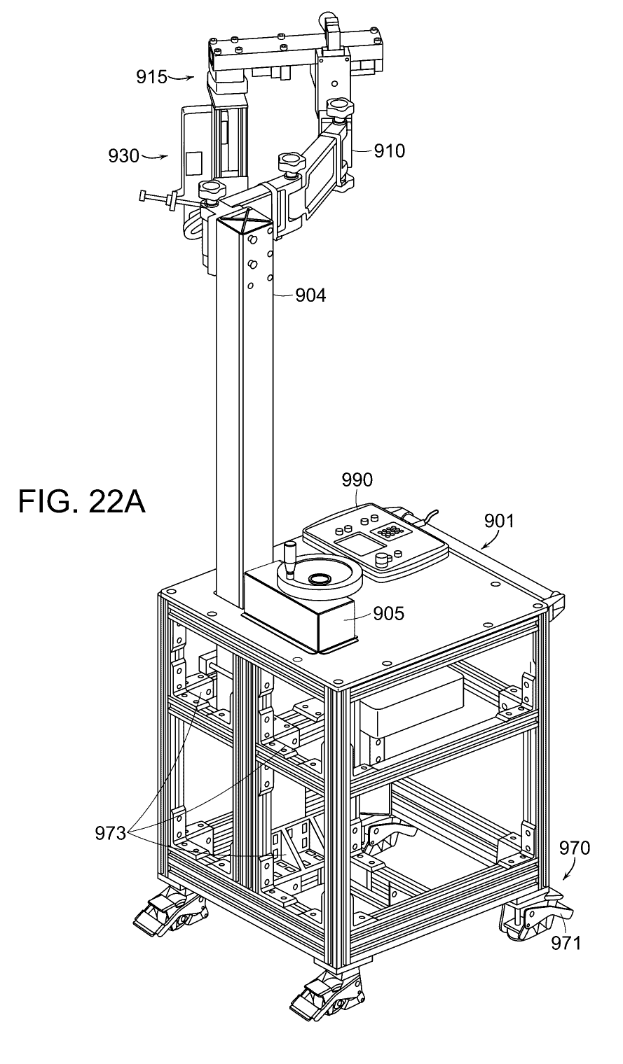

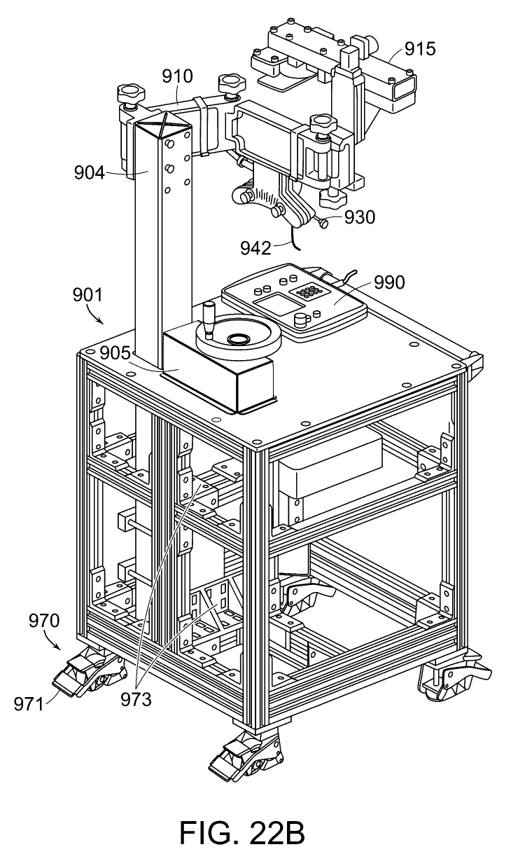

FIGS. 3A and 3B respectively show front view and side view images a rotary friction drive 203 and related parts according to some embodiments. Rotary friction drive 203 includes a drive gear 302, a mechanism to grip the delivery catheter (such as one or more Viton O-rings) 301, a luer-lock connection/mounting bracket 303. In one embodiment, the delivery catheter is gripped between two Viton O-rings 301 and advanced or retracted by using the drive gear 302 on the rotary friction drive 203. A programmable motor controller 990 (see FIG. 22B) may power the rotary friction drive 203 and control the advancement and retraction of the delivery catheter 943. In some embodiments, the mounting bracket 303 on the rotary friction drive 203 secures the rotary friction drive 203 to the stereotaxic assembly 204 (not shown). In some embodiments, the rotary friction drive 203 may be positioned away from the stereotaxic assembly.

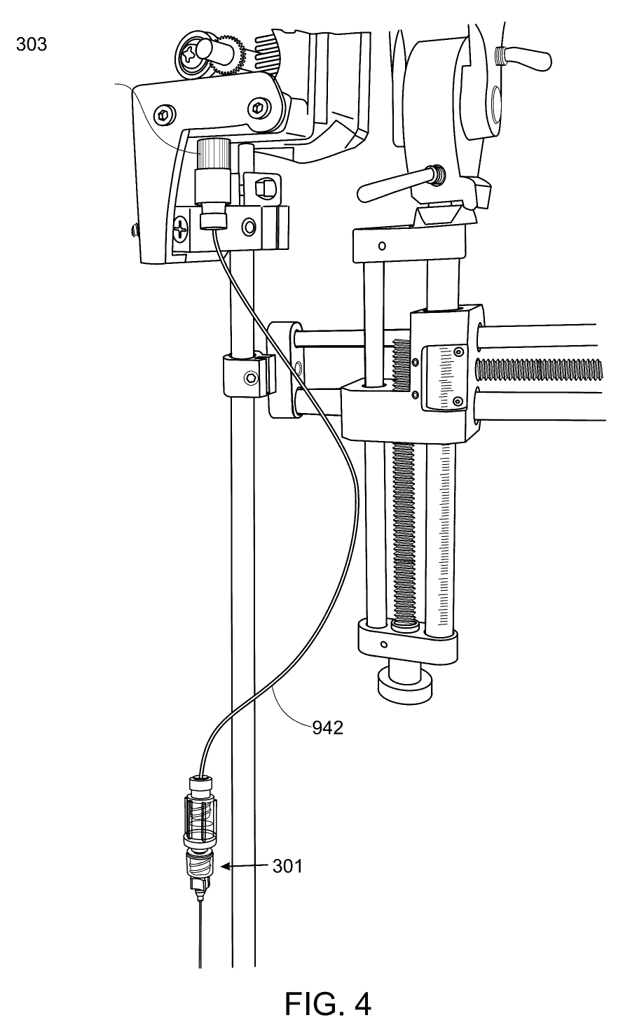

FIG. 4 shows an image of an upper section of a cell trail injection system according to an embodiment. In particular, FIG. 4 shows the connection between the guide tubing 942 and the rotary friction drive 203, the guide tubing 942, and the attachment between the combined guide tubing and the proximal end of the introducer needle 942. The luer-lock connection 301 at the bottom of the rotary friction drive 203 may enable the connection of guide tubing 942. In one embodiment, a luer-lock connection on the introducer needle 842 may enable connection of the guide tubing to form a unitary two-part guide tube 94. This secure guide tubing connection 301 between the rotary friction drive 203 and the introducer needle (or sometime referred to as a guide needle or guide tube interchangeably) 942 may be used for the controlled advancement of the delivery needle 943. In some embodiments, in the absence of a secure and closed tubing connection between these two components, the delivery catheter may buckle during advancement into the spinal cord or other anatomical space. This buckling of the delivery needle may prevent the controlled linear actuator-driven entry of the delivery needle into the spinal cord or other anatomical space.

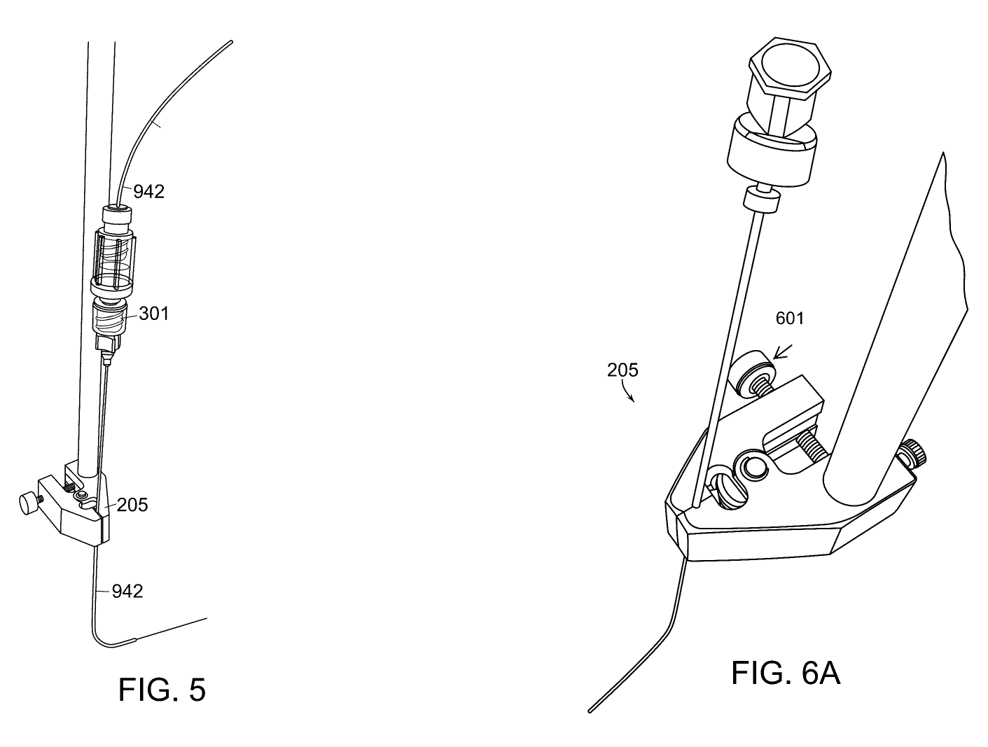

FIG. 5 shows an image of a lower section of a cell trail injection system according to an embodiment. In particular, FIG. 5 shows the introducer needle attached on its proximal end to the guide tubing through a luer-lock connection. The introducer needle is held by an guide tube/introducer needle holder.

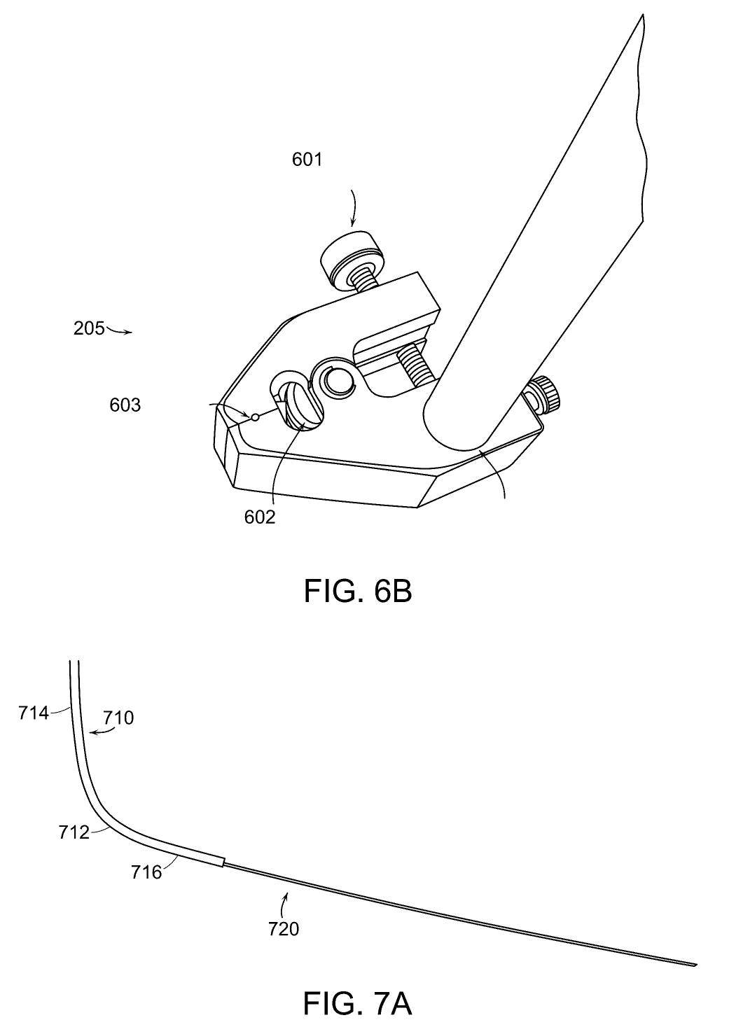

FIG. 6A-6B show images of an guide tube/introducer needle holder 205 in a cell trail injection system according to some embodiments. Introducer guide tube/needle holder 205 includes a thumb screw, a needle grip, a spring, and a connection to the stereotaxic positioner. The guide tube/introducer needle holder may securely grip the guide tube/introducer needle and link the guide tube/introducer needle 942 to the stereotaxic positioning apparatus 204. This structure may enable a more precise positioning of the guide tube/introducer needle 942 on the spinal cord. The spring mechanism in the guide tube/introducer needle holder 205 may facilitate the loading and release of the guide tube/introducer needle 942. The spring mechanism 602 may also allow for rotation of the guide tube/introducer needle 942 without removing the guide tube/introducer needle from the assembly. Rotation of the guide tube/introducer needle 942 may be used for accurately aligning the path of the delivery catheter with the axis of the spinal cord. Once the guide tube/introducer needle is appropriately angled, the thumb screw 601 on the guide tube/introducer needle holder 205 may be tightened to secure the introducer needle in place by needle grip 603.

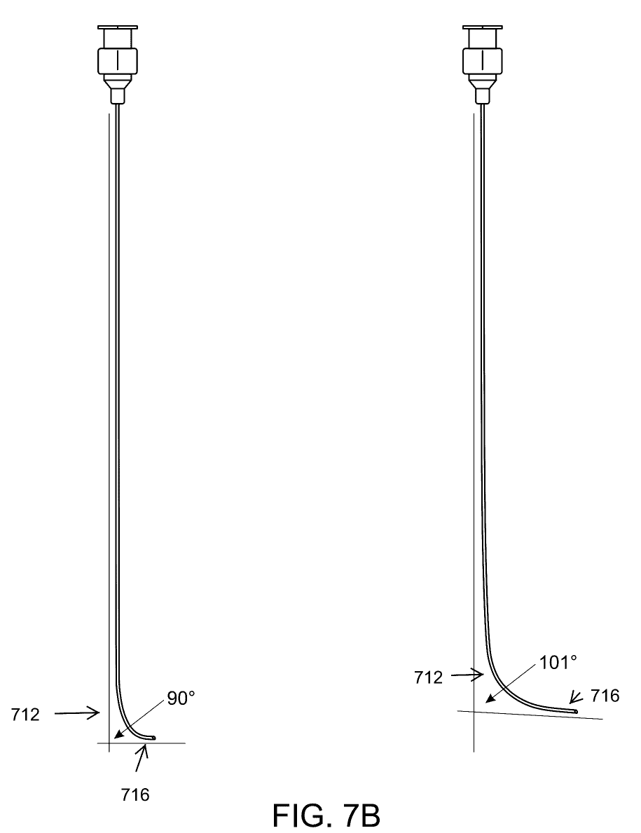

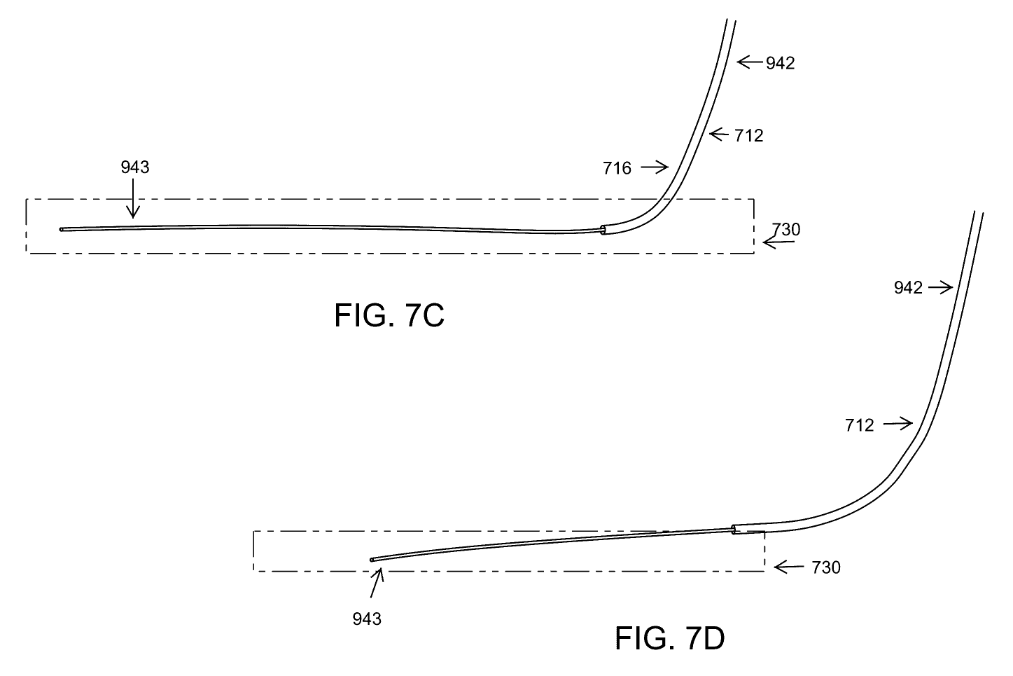

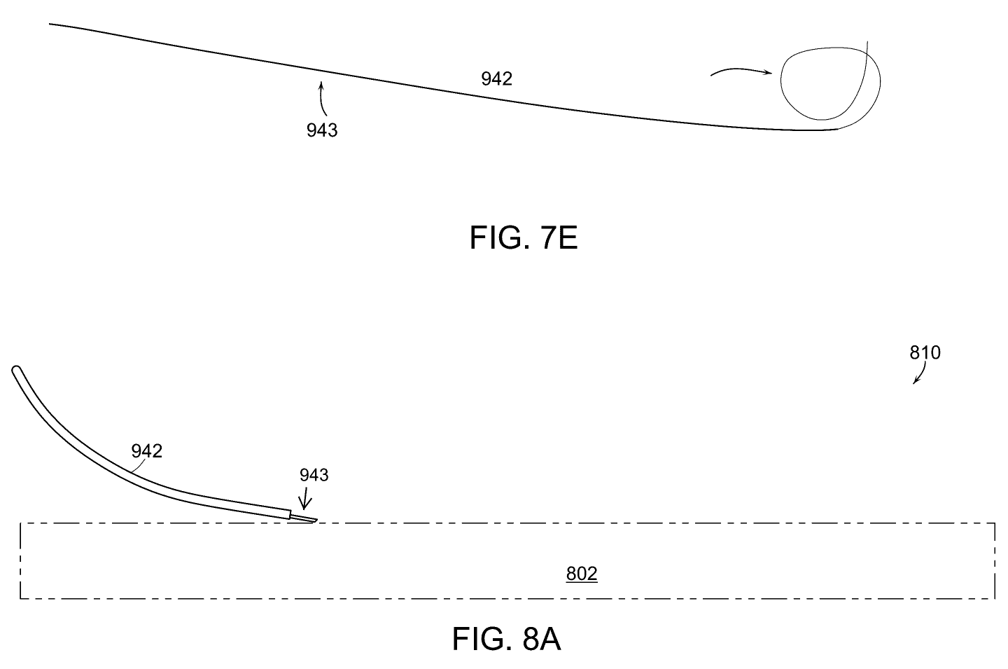

FIG. 7A-7E show images of guide tube/introducer needles 942 and delivery catheters/needles 943 according to various embodiments. FIG. 7A shows a delivery catheter 720 protruding from the introducer needle 710. Introducer needle 710 has a curved section, or bend, 712 near its distal end 716. The bend may cause the direction of the distal end of the delivery needle to differ from the direction of the needle before the bend 714. The direction of the needle at a point may be defined as the direction of a line that is tangent to the delivery needle at that point.

The bend in the guide tube/introducer needle may characterized by the angle between the direction of the needle before and after the bend. In some embodiments, this angle is also known as an angle between a proximal portion 714 (a portion of the needle before the bend) and a distal portion 716 (a portion of the needle between the bend and the distal end).

FIGS. 7B-7D show introducer needles with two different bends. In a 90 degree bent needle, the angle is around 90 degrees, while in a 101 degree bent needle, the angle is around 101 degrees. The bend may facilitate positioning of the distal end of the guide tube/introducer needle and introduction of the delivery catheter into the spinal cord 720. Moreover, the bend 712 may determine the direction or length of the trail inside the spinal cord. A 90 degree bent needle may be particularly suitable for injections parallel to the cord or subpial injections.

A trail of a therapeutic cells and/or one or more therapeutic substances or diagnostic substances or injectable medium may be delivered parallel to the spinal cord (parallel trail) or at an angle within the cord (angled trail). In the parallel trail method, a parallel therapeutic trail may be created by inserting an guide tube/introducer needle with a 90 degree bend into the cord or beneath the pia matter (i.e., a subpial injection), resulting in the extruded delivery needle to exit the guide tube/introducer needle in a parallel path with respect to the spinal cord. Because the spinal cord is a soft and highly vascularized tissue, one concern in such operations is damaging the spinal cord during entry of the guide tube/introducer needle. Generating a parallel trail to the cord by inserting a 90 degree bent introducer needle may be accomplished by creating a small dorsal myelotomy and lowering the guide tube/introducer needle into the cord. This procedure, however, may pose safety concerns due to the risk of damaging the cord. In some embodiments, it may also be difficult to quickly and safely remove the introducer needle in case of an adverse event during injection. A subpial injection, without inserting the guide needle into the cord, may reduce damage to the cord parenchyma.

Some embodiments use the angled trail method in which, instead of a parallel therapeutic trail, an angled therapeutic trail is delivered. In order to deliver an angled therapeutic trail, an guide tube/introducer needle with an obtuse angle (i.e., an angler that is larger than 90 degrees, such as the 101 degree needle of FIG. 7B) may be placed on the surface of the spinal cord. When the delivery needle exits the guide tube/introducer needle, it will enter the spinal cord at a small acute angle with respect to the spinal cord. This set up may result in an angled trail within the cord. The length and direction of the trail can be defined by the angle of the guide tube/introducer needle. Angles closer to 90 degrees may yield longer and shallower trails compared to angles closer to 180 degrees (which creates a straight down perpendicular trail across the cord). In some embodiments, the angled trail method has the advantage that the introducer needle does not need to be inserted into the spinal cord, thus reducing the risk of damaging the spinal cord. Furthermore, in case of an adverse event, the delivery catheter may be rapidly retracted back into the guide tube/introducer needle and the guide tube/introducer needle can be rapidly raised away from the spinal cord.

FIG. 7E shows an image of a delivery catheter according to an embodiment. In FIG. 7E the delivery needle 943 is made of nitinol, which is a superelastic shape memory alloy. The superelastic property of nitinol allows it to revert back to its pre-programmed shape after deformation. In one embodiment, the nitinol is programmed or annealed with a straight shape. When used for delivery, when passing through the bent guide tube/introducer needle, the nitinol needle deforms into the bent shape. Upon exiting the guide tube/introducer needle, however, the nitinol needle reverts back to its pre-programmed straight shape. This reshaping is utilized for creating a straight therapeutic trail. A needle made of non-superelastic materials, such as stainless steel, may permanently deform in the curved introducer needle and create a curved or deformed trail within the spinal cord. Furthermore, passing a curved delivery needle through the spinal cord may result in tissue damage.

In various embodiments, the delivery catheter 943 may have a beveled, curved, or blunt distal end. The direction of the beveled end in relation to the cord may serve to affect the trajectory of the delivery needle once it is within the spinal cord. Steering the delivery catheter by rotating the beveled end may be used to avoid blood vessels or target a defect site. Rotation of the beveled end of the delivery catheter may be accomplished by torqueing or rotating the delivery catheter at a point between the guide tube/introducer needle and the linear actuator or above the linear actuator. A grip affixed to one of these positions may facilitate rotation of the bevel.

In some embodiments, a blunt or curved end of the delivery catheter may result in safety advantages. A blunt or curved end of the delivery catheter may push past blood vessels rather than puncturing them, in turn reducing the risk of hemorrhage within the spinal cord. Furthermore, in some embodiments, pushing through tissue may cause less damage compared to cutting tissue with a sharp end.

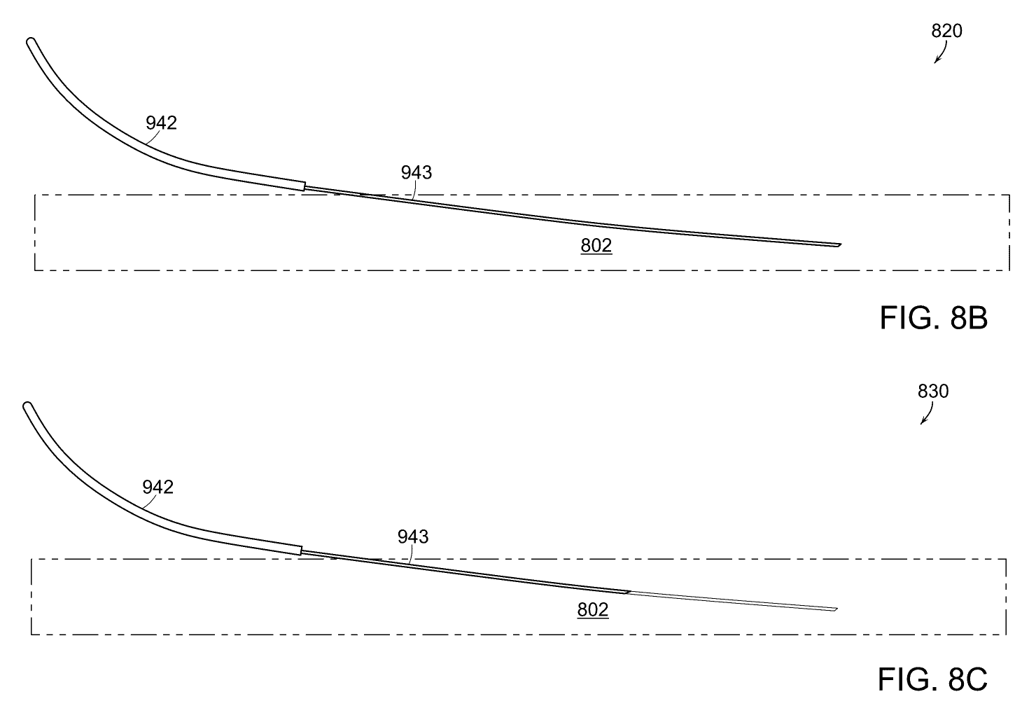

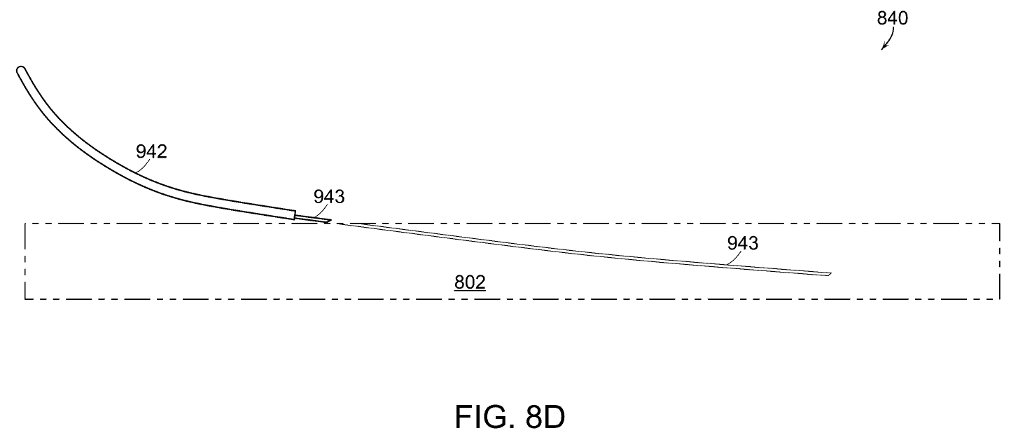

FIGS. 8A-8D show images of four steps (810, 820, 830, and 840) of creating a therapeutic trail in an experimental medium constituting an anatomical space 802 by a therapeutic trail delivery system according to one embodiment. In particular, in steps 810-840, a curved introducer needle 943 introduces a delivery needle 942 into an experimental medium 802, and the delivery needle creates a trail 808 in medium 802. In FIGS. 8A-8D, experimental medium 802 is a spinal cord mimetic gel.

More specifically, in step 810, guide tube/introducer needle 804 is positioned on the surface of medium 802.

In step 820, delivery catheter 943 is passed through guide tube/introducer needle 942 and introduced into medium 802. The motion of delivery catheter 943 may be under control of a linear actuator or rotary friction drive. In this example the delivery catheter 943 is introduced at an acute 11 degree angle with respect to the surface of the spinal cord mimic 802. The delivery catheter 943 is extruded a distance (here 4 centimeters) inside medium 802. The syringe pump 201 (see FIG. 2) controlled flow of therapeutic cells and/or one or more therapeutic substances or diagnostic substances or injectable medium (in this example: neural stem cells in a hyaluronic acid carrier) may be initiated once the delivery needle is fully extruded into the medium. This delivery of therapeutic cells and/or one or more therapeutic substances or diagnostic substances or injectable medium may couple with the automated retraction of the delivery catheter.

In step 830 delivery needle 943 is partially retracted back into guide tube/introducer needle 942. During the retraction, a therapeutic trail is generated along the track of the delivery needle, as visible in FIG. 8C to the right of the delivery catheter.

In step 840, delivery needle 943 has retracted out of medium 802 and back into guide tube/introducer needle 942. A homogenous therapeutic trail is visible within the medium. No cellular reflux is visible at the top of the medium. Cellular reflux is cell suspension that does not deposit within the experimental medium. Therefore, the presence of cellular reflux would be visible as a volume of cell suspension at the top of the experimental medium (spinal cord mimic), near the entry point of the delivery needle.

In some embodiments, the trail injection system is mounted to the operating table or a cart that comes up to the patient. Then the guide tube/introducer needle is lowered into the surgical field, respiration is halted, and the trail is created. In some embodiments, respiration needs to be halted because the spine moves during respiration. This motion may cause damage to the spinal cord during trail creation.

In some embodiments, the trail creation injection system, including one or more of the stereotaxic apparatus 204, guide tube/introducer needle 942, delivery catheter/needle 943, and the linear actuator or rotary friction drive 203 may be secured to the patient's spine (spine mounted). In such embodiments, during respiration the device would move with the patient because it secured to the plane of motion (spine) rather than to an immobile object (table). Such embodiments with the spine-mounted (aka floating) approach, may not need to stop respiration during injection.

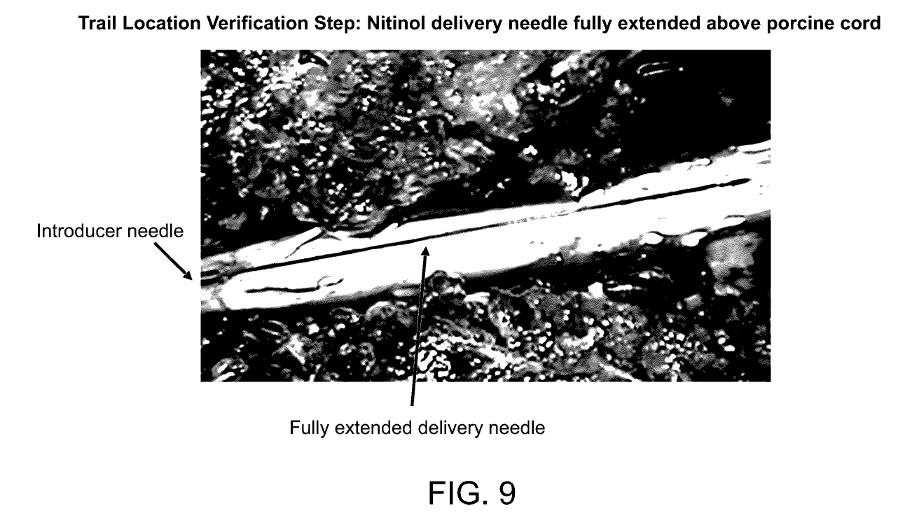

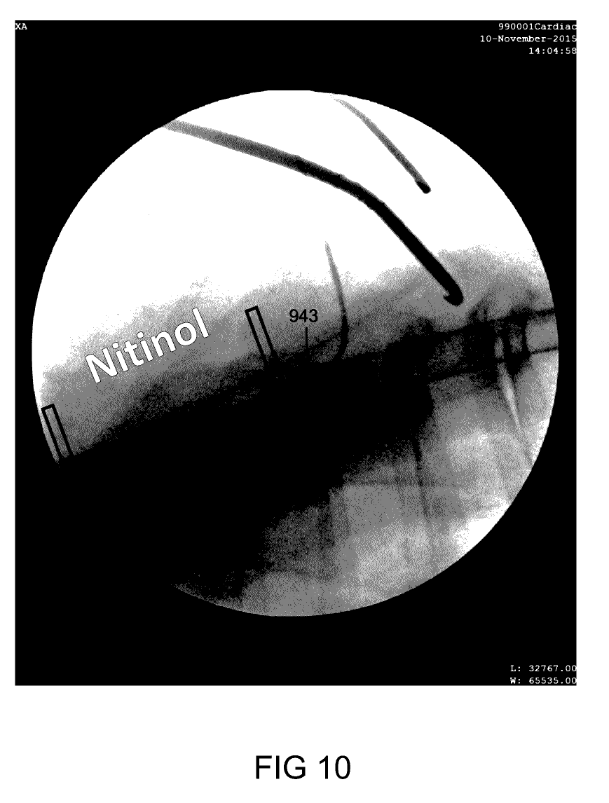

FIGS. 9 and 10 demonstrate some aspects of the surgical procedure involved in creating therapeutic trails in a spinal cord according to various embodiments. In these figures, the trails were created in a porcine spinal cord.

FIG. 9 shows a trail alignment step according to an embodiment. In the alignment stage, the location and extension of the trail is verified before the delivery needle is inserted into the spinal cord. FIG. 9 shows an image of a nitinol delivery needle fully extended and aligned above a porcine spinal cord. The trajectory of the delivery needle may be verified prior to introducing the delivery needle into the parenchyma of the spinal cord. This may be accomplished by fully extruding the delivery catheter above the spinal cord, rather than within the spinal cord. The guide tube/introducer needle may then be rotated within the guide tube/introducer needle holder in order to align the delivery needle. Once alignment is confirmed, the delivery catheter may be fully retracted back into the guide tube/introducer needle and the introducer needle may be lowered to the surface of the cord, in preparation of inserting the delivery catheter into the spinal cord.

In some embodiments, before inserting the delivery catheter into the spinal cord, ventilation of the patient may be suspended. This suspension may help prevent motion-induced damage of the spinal cord. After that, the stepper module may drive the delivery catheter out of the guide tube/introducer needle to enter into the spinal cord.

FIG. 10 shows a myelogram of a nitinol delivery needle 943 extended in a porcine spinal cord according to one embodiment. In FIG. 10, fluoroscopy coupled with an x-ray contrast agent in the subarachnoid space (myelogram) was used to demarcate the boundaries of the spinal cord. Imaging techniques such as fluoroscopy or ultrasound may be used to visualize the spinal cord and the path of the delivery needle 943. These techniques may confirm the position of the trail and prevent the delivery needle from puncturing the ventral aspect of the spinal cord.

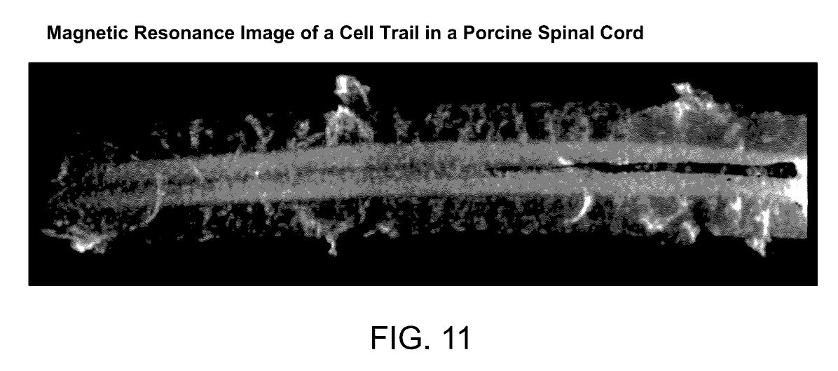

FIG. 11 shows a T1-weighted magnetic resonance image of a 4 cm human neural stem cell trail created in a porcine spinal cord according to one embodiment. The trail is false-colored to improve visibility.

Some embodiments utilize therapeutic cells and/or one or more therapeutic substances or diagnostic substances or injectable medium that includes shear-thinning polymers or viscous liquids. In some embodiments, the shear-thinning polymers or viscous liquids prevent aggregation or settling of the therapeutic elements, such as cells, that are also included in the therapeutic cells and/or one or more therapeutic substances or diagnostic substances or injectable medium.

In some therapeutic injection systems, the therapeutic cells and/or one or more therapeutic substances or diagnostic substances or injectable medium is a suspension that includes the therapeutic elements. These elements may rapidly aggregate or settle. This poses a problem when the delivery of a uniform suspension is necessary in applications such as cell therapy, 3-D printing/tissue engineering, etc.

The settling of the elements may also pose a problem when transporting pre-loaded syringes of cells. In therapeutic delivery applications, including cell delivery or the creation of therapeutic trails of cells, cells may settle in the delivery syringe prior to or during injection. This may result in problems such as inaccurate dosing, inhomogeneous cell delivery, and potentially cell death during injection. This problem is exacerbated in cell delivery applications where the delivery needle is so long that the duration of the operation is comparable with the settling time of the element.

The shipping of containers, such as a syringe, pre-loaded with a cell suspension may be difficult due to cell settling during the shipping process. To circumvent this problem, additional handling steps may be required to prepare the cells prior to administration at the time of surgery. Preventing or reducing settling or aggregation of the therapeutic elements, such as cells, will therefore address or reduce the effect of the above-discussed problems. Reducing settling may include prolonging the characteristic settling time for the elements.

Various embodiments use a settling reduction technique to address the settling or aggregation problems. The settling reduction techniques may be used when a uniform cell dispersion is required for period that may be 30 seconds, 1 minute, 5 minutes, or longer. This technique may also be used to transport therapeutic elements, such as cells, pre-loaded in a syringe, to maintain the homogeneity of the cells during shipping.

Various embodiments employ a settling reduction technique by creating a suspension of the therapeutic elements (e.g., cells) in a viscous liquid or shear-thinning polymer such as hyaluronic acid. The vicious liquid or shear thinning polymer may be formulated in a divalent ion-free buffer solution such as phosphate buffered saline. In some embodiments the weight percentage of hyaluronic acid in the divalent ion-free carrier may be from 0.5 wt. % to 1 wt. %. The average molecular weight of the hyaluronic acid may be larger than 1000 kDa (700 KDa to 1,900 KDa.). Compositions and method s for preparing and injecting trails of therapeutic cells and/or one or more therapeutic substance or diagnostic substance or other injectable medium are described in co-pending application filed on the same date herewith as U.S. non-provisional application Ser. No. ______, entitled COMPOSITIONS AND METHODS FOR PREPARING AN INJECTABLE MEDIUM FOR ADMINISTRATION INTO THE CENTRAL NERVOUS SYSTEM filed on the same date as the present application, the entire contents of which are incorporated herein by reference.

In some embodiments, the viscosity of the solution is tuned such that the viscosity prevents the settling and aggregation of the elements but does not interfere with cellular survival, migration, and outgrowth of cell projections or neurites, in the case of neural stem cells or neurons. In some embodiments, a solution that is too viscous may block the migration and outgrowth of cells and compromise the integration into host tissue. Further, in some embodiments, a solution that is too viscous may limit the diffusion of nutrients to the transplanted cells and compromise their viability.

Moreover, in some embodiments, the viscosity of the solution is tuned to maintain adequate handling characteristics. These characteristics may include ease of mixing with cells, preventing bubble formation, ease of injection, etc. The viscosity of the solution may also prevent efflux of the cells when injected into a confined tissue space. In some embodiments, the settling reduction technique creates a therapeutic substance, in the form of a suspension of the therapeutic elements, e.g., cells, in which the suspension remains stable and uniform for greater than 24 hours.

In some embodiments, the mechanical properties of the cell carrier may be determined by measuring its storage modulus by rheology. In some embodiments, the storage modulus of the cell carrier may be greater than 10 Pa or 50 Pa but lower than 500 Pa. In some embodiments, the storage modulus is between 10 Pa and 50 Pa.

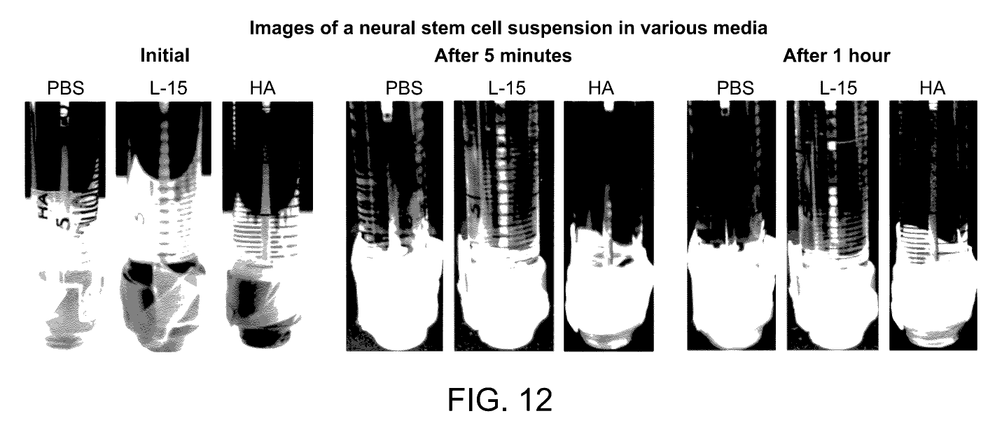

FIG. 12 shows images of neural stem cells suspended in various media for up to one hour according to one embodiment. FIG. 12 depicts that the cells may settle in phosphate buffered saline (PBS) after 5 minutes and may aggregate in Leibovitz L-15 medium (L-15) within 5 minutes. In a divalent cation-free hyaluronic acid suspension (0.5 wt. % hyaluronic acid in this embodiment), however, the cells are uniformly suspended for up to one hour. Some embodiments utilize this property for the delivery of a homogenous cell suspension when the delivery time is up to one hour or potentially longer.

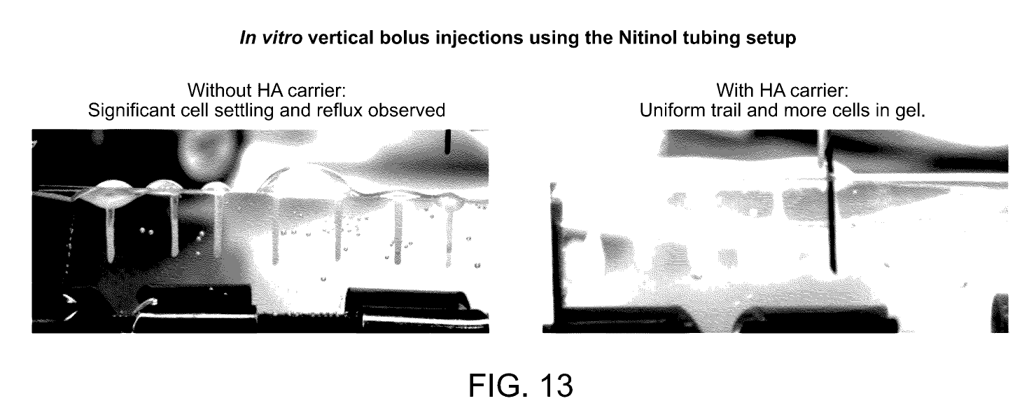

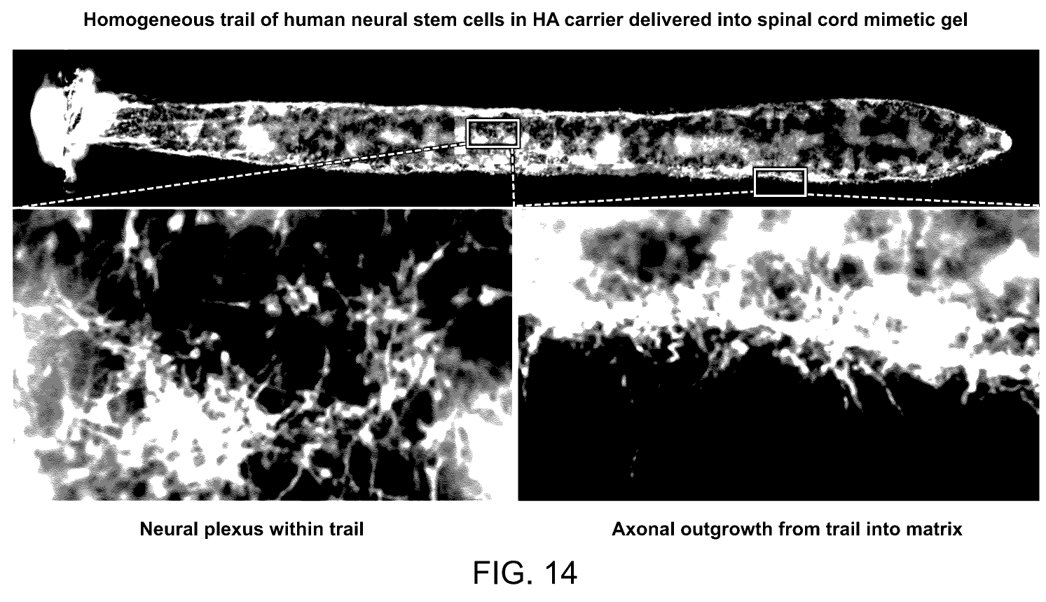

FIG. 13 depicts the delivery of neural stem cells into a spinal cord mimetic gel according to an embodiment. When the cells are delivered without a hyaluronic acid carrier (in L-15 medium), cells may aggregate to the bottom of the needle track. When cells are injected in an hyaluronic acid carrier (0.75 wt. % hyaluronic acid in a divalent ion-free PBS), however, the cells are uniformly distributed along the injection track.

FIG. 14 depicts, according to an embodiment, microtubule associated protein-2 (MAP2) staining of human neural stem cells in a hyaluronic acid carrier injected into a spinal cord mimetic gel in vitro. The cells express survive and pre-neuronal markers in the uniform cell trail. Outgrowth of neuronal projections is also visible at the boarders of the trail. In some embodiments, this situation indicates that the viscosity of the hyaluronic acid formulation (0.75 wt. %) permits the survival and outgrowth of human neural stem cells in vitro.

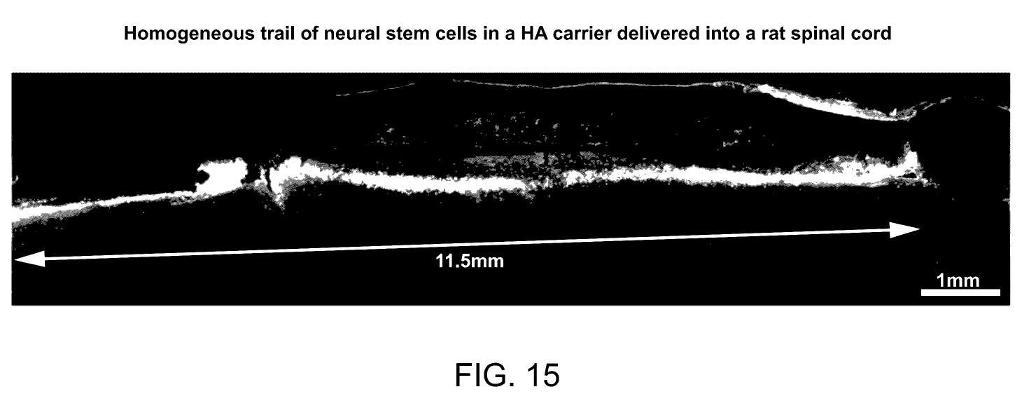

FIG. 15 shows a trail of neural stem cells in a hyaluronic acid carrier delivered into a rat spinal cord according to an embodiment. In this embodiment, the hyaluronic acid formulation facilitated a homogenous cell trail in vivo and the viscosity of the hyaluronic acid solution permitted cell survival.

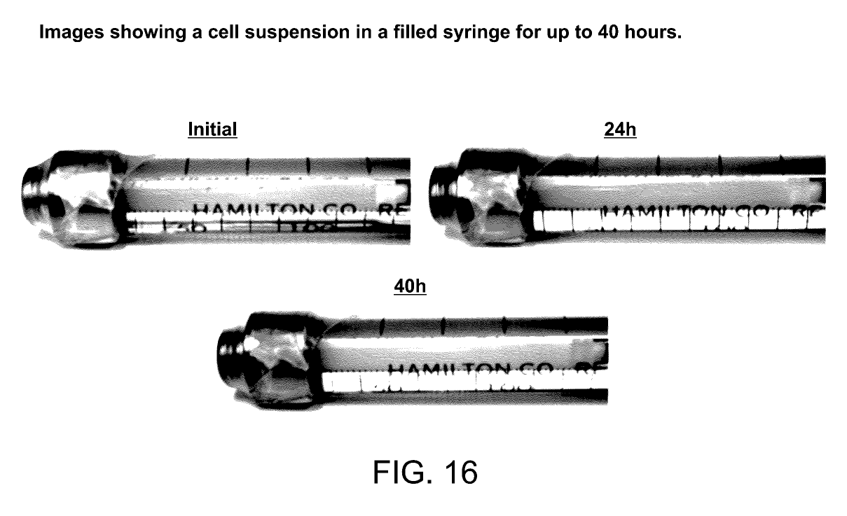

FIG. 16 shows images of neural stem cells in hyaluronic acid (0.75 wt. %) stored in a syringe for up to 40 hours according to an embodiment. This time course may be representative of the time necessary to ship a pre-filled syringe of cells to the site of application (for example, hospital). The images in FIG. 16 show that a homogenous cell suspension may be maintained in hyaluronic acid for up to 40 hours and demonstrate the utility of this formulation as a cell carrier for shipping applications.

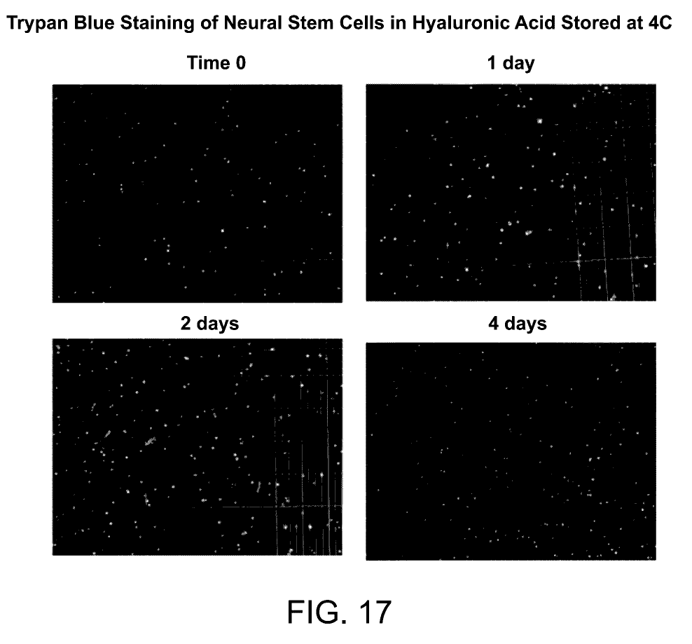

FIG. 17 shows trypan blue staining of rat neural stem cells stored in a 0.75 wt. % hyaluronic acid carrier at 4 C according to one embodiment. This time course of images may demonstrate that the cells, shown as bright spots, may remain viable over the course of 4 days when stored in the hyaluronic acid. This result may indicate that the hyaluronic acid weight percentage and molecular weight used, correlating to a solution storage modulus of ˜10 Pa, may maintain the viability of cells and may be suitable for the maintaining the viability of cells. The homogenous cell suspension shown in FIG. 16 coupled with the cell viability shown in FIG. 17 may demonstrate the utility of the viscous liquid or shear-thinning polymer for cell transportation.

Particular alternative embodiments of the injection device of the present invention will be described.

### Injection Device

In various embodiments of the present invention, the injection device may comprise all or a subset of the elements depicted in FIG. 2 and FIG. 18.

In a certain embodiment, an injection system for delivering a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord or on the surface of the spinal cord parenchyma, may comprise: a) at least one linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the injection needle subassembly comprises a first telescoping guide needle having an inner cannula and an outer diameter; and a second cannula having a second inner cannula slidably engaged with the outer diameter of the first telescoping guide tube/needle; a delivery catheter/injection needle inserted through the first inner and second inner cannulas and connecting at one end with the pre-filled syringe needle connector and formed into a needle point at the opposite end; wherein the delivery catheter is secured to the interior surface of the second rigid cannula; and wherein the second cannula and the plunger rod are connected to the linear actuator; c) a micro-positioning subassembly for orienting the flexible wire catheter in the x, y and z axes relative to a prone animal or human positioned under the injection device; and d) a programmable controller capable of controlling the linear actuator to (i) advance and retract the injection needle and (ii) to control the volume and flow rate of the contents of the pre-filled syringe through actuation of the plunger rod in the operation of the injection device. In another embodiment, the macro-positioning subassembly may comprise a goniometer comprising a micro-angular adjustment and optionally a macro-angular adjustment.

### Injector Device Subassembly

With reference to the Figures accompanying this description, the skilled person can readily assemble or obtain an injector device subassembly for actuating (1) a separately provided injection needle subassembly and a (2) a separately provided pre-filled syringe containing therapeutic cells and/or one or more therapeutic substances or diagnostic substances or injectable medium.

### Prefilled Syringe

An injection syringe as described in this disclosure and accompanying figures may readily be obtained with a needle connector at one end and a plunger attached to a plunger rod at the opposite end. An exemplary syringe is a Hamilton syringe comprising a glass barrel and a removable needle (RN) assembly. Alternatives embodiments may include a syringe fitted with a Luer Lock fitting. The syringe maybe sterilized by conventional means and filled under aseptic conditions with therapeutic cells and/or one or more therapeutic substances or diagnostic substances or injectable medium, as described elsewhere in this disclosure. Alternatively, the pre-filled syringe could be filled with a sterile suspension, sterile solution, sterile emulsion, or other suitable pharmaceutical composition comprising one or more therapeutic substances such as a growth factor, antibody, analgesic, anesthetic and the like.

### Injection Needle Subassembly

In accordance with the disclosure set forth herein and the accompanying Figures, the skilled person could fabricate or obtain, a first telescoping guide needle having an inner cannula and an outer diameter; and a second rigid cannula having a second inner cannula capable of being slidably engaged with the outer diameter of the first telescoping guide needle; a delivery needle inserted through the first inner and second inner cannulas and connecting at one end with the pre-filled syringe needle connector and formed into a needle point at the opposite end; wherein the delivery catheter/injection needle is secured to the interior surface of the second cannula by, for example, an epoxy adhesive; and wherein the second cannula is suitable for connection to a linear actuator. Such telescoping assemblies could be manufactured to be disposable following use.

In another embodiment, the injection needle sub-assembly may comprise: (i) a flexible delivery catheter, comprising a flexible wire cannula or a cannula comprising a polymeric substance, comprising a syringe needle connector capable of attaching to the needle connector of the pre-filled syringe at one end and having a needle point or smooth or blunt tip at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide needle. The telescoping two-part slide mechanism operates on a similar principle to a trombone slide.

The delivery catheter/injection needle is dimensioned to pass through the telescoping two-part slide mechanism. The delivery catheter is secured to the interior of the outer cannula thereby providing for vertical movement of the outer cannula and attached delivery catheter upon actuation of the connected linear actuator. In some embodiments, the delivery catheter is capable of forming a service loop at the end of the catheter attached to the prefilled syringe.

The outer cannula may be attached to a first mounting block that connects to a linear rail. The first linear actuator upon rotation results in actuation of the linear rail which moves the mounting block forward and backward. The inner cannula is attached to a second mounting block that rigidly connects to the injection needle subassembly connector of the injector device subassembly.

The telescoping two-part slide mechanism may be fabricated from 316 stainless steel and the delivery catheter may be fabricated from nitinol (nickel-titanium alloy, oxide finish) 29 gauge catheter, in a preferred embodiment, which tubes and catheters are available from multiples sources. Alternative metallic tubes and flexible wire catheters may be utilized as would be evident to a person skilled in the art. In addition, the catheter may be formed by a medically acceptable, natural or synthetic polymeric substance, for , example, a polyester such as polyethylene.

### Micro-Positioning Subassembly

In an embodiment, the micro-positioning subassembly permits orientation of the delivery catheter in the x, y and z axes relative to an animal or human positioned adjacent the injection device. In another embodiment, the micro-positioning subassembly may comprise a goniometer comprising a micro-angular adjustment and optionally a macro-angular adjustment. In yet other embodiments, the positioning subassembly further comprises a vertical height adjustable post, an adjustable articulated arm and in yet other embodiments a micro-angular adjustment. In further embodiments the micro-positioning subassembly further comprises: a first horizontal support arm; a second horizontal support arm oriented at right angles to the first horizontal support arm; and a rotatable stage member; wherein the micro-positioning subassembly further comprises: a first horizontal support arm; a second horizontal support arm oriented at right angles to the first horizontal support arm; and a rotatable stage member; wherein the first horizontal support arm comprises one or more adjustable vertical support rail attached to a first vertical support rail micro-adjustor for adjusting the first horizontal support arm along the z axis; further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; and further wherein the goniometer is mounted on one or more rails attached at the top of the goniometer rail to the bottom surface of the rotatable stage. The goniometer permits adjustment of the guide tube/needle about its distal end or tip. In some embodiments the rotatable stage member rotates about the tip of the guide tube/needle.

### Programmable Controller

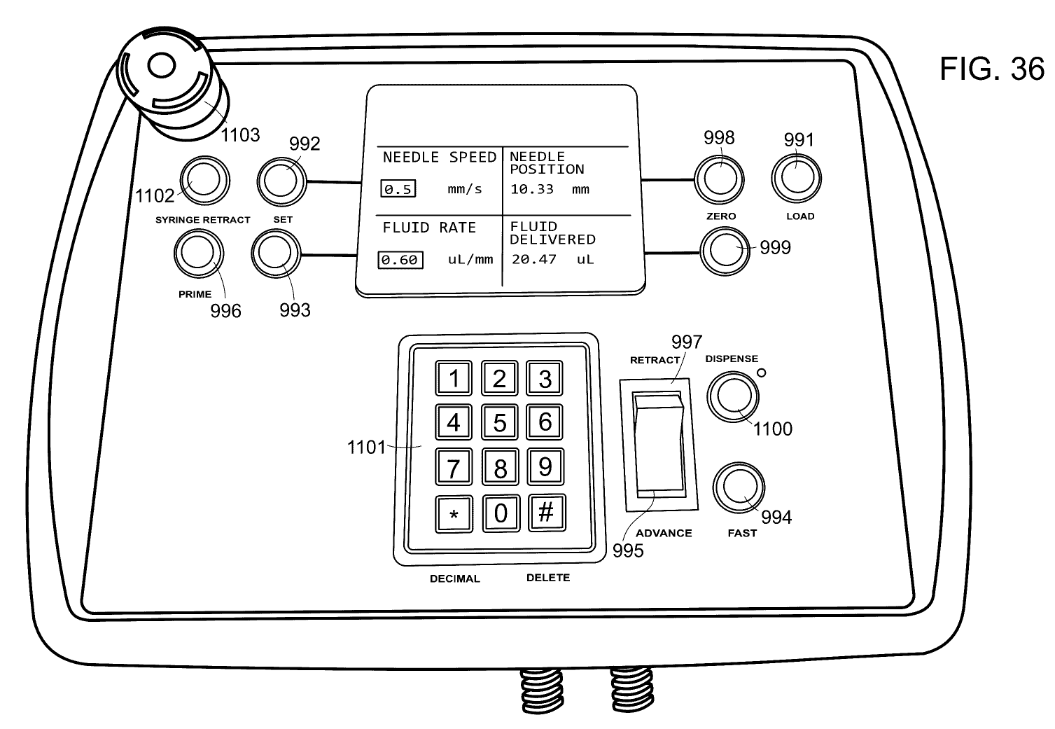

In an embodiment a controller capable of controlling the linear actuator is employed to (i) advance and retract the injection needle and (ii) to control the volume and flow rate of the contents of the pre-filled syringe through actuation of the plunger rod in the operation of the automated injection device. The skilled person will understand how to assemble such a programmable controller to carry out the functions described in connection with FIG. 36 below.

### Micro-Angular Adjustment Mechanism:

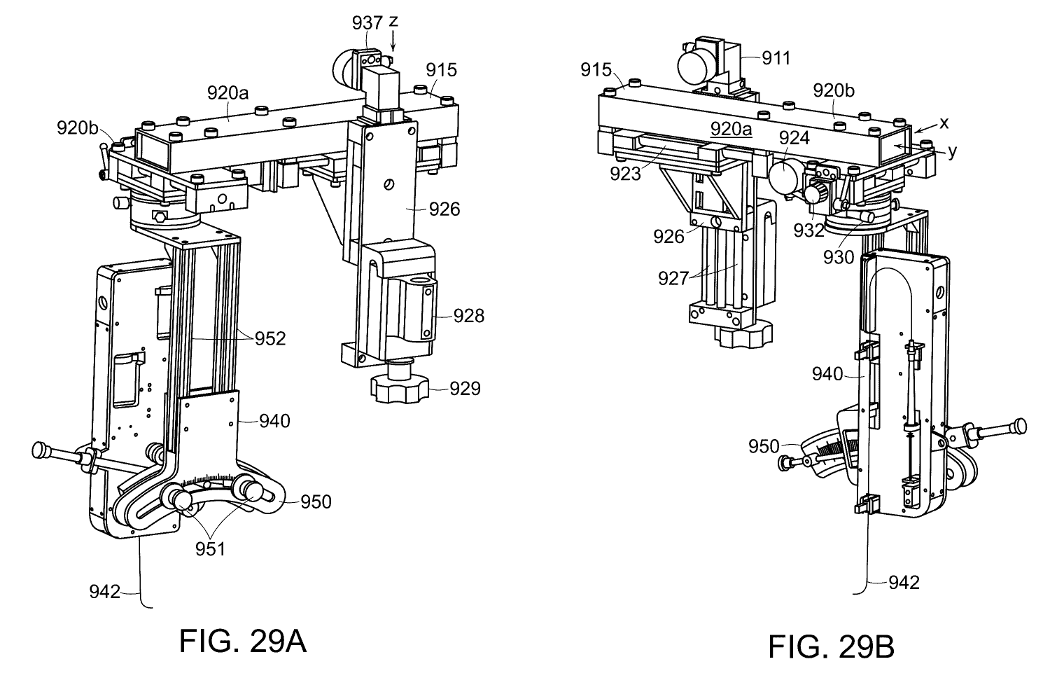

With specific reference to FIG. 29, a micro-angular adjustment mechanism (also referred to as an XYZ mounting system herein) 915 in an embodiment of the present invention may be constructed in the following manner. The micro-angular positioning subassembly may comprise: a first horizontal support arm 920a; a second horizontal support arm oriented at right angles to the first horizontal support arm 920a; and a rotatable stage member 930; wherein the first horizontal support arm comprises one or more adjustable vertical support rail 927 attached to a first vertical support rail micro-adjustor 911 for adjusting the first horizontal support arm along the z axis. The micro-angular adjustment mechanism may further comprise a first horizontal rail attached to a first horizontal rail micro-adjustor 924 for adjusting the first horizontal rail in the y axis. The micro-angular adjustment mechanism may further comprise a second horizontal support arm 920b and a horizontal support arm rail attached to a second horizontal support arm micro-adjustor 931 for adjusting the second horizontal support arm in the x axis. A rotatable stage 930 is included in an embodiment which has a top surface and a bottom surface. The top surface is attached to the underside of the second horizontal support arm and the rotatable stage 930 has a bottom surface. In a further embodiment, a goniometer 950 is mounted on one or more rails 952 attached at the top of the goniometer rail to the bottom surface of the rotatable stage 930.

The foregoing and other embodiments of the present invention may be understood with reference to the following description, exemplary embodiments and FIGS. 18 to 52 below.

Embodiment 1: An injection device for trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord or on the surface of the spinal cord parenchyma below the pia mater , comprising: a) at least one linear actuator; b) an injector device sub-assembly for actuating (1) a separately provided injection needle subassembly and a (2) a separately provided syringe containing therapeutic cells and/or one or more therapeutic substances or diagnostic substances or injectable medium; wherein the plunger rod is actuated by the at least one linear actuator; wherein the injection needle subassembly comprises a first telescoping guide needle having an inner cannula and an outer diameter; and a second cannula having a second inner cannula slidably engaged with the outer diameter of the first telescoping guide needle; a delivery catheter/injection needle inserted through the first inner and second inner cannulas and connecting at one end with the pre-filled syringe needle connector and formed into a needle point at the opposite end; wherein the delivery catheter is secured to the interior surface of the second rigid cannula; and wherein the second rigid cannula and the plunger rod are connected to the at least one linear actuator; c) a macro-positioning subassembly for orienting the delivery catheter in the x, y and z axes relative to an animal or human positioned adjacent the injection device; and d) a programmable controller capable of controlling the at least one linear actuator to (i) advance and retract the injection needle and (ii) to control the volume and flow rate of the contents of the pre-filled syringe through actuation of the plunger rod in the operation of the injection system.

Embodiment 2: An injection device for delivering a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) at least one linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by the at least one linear actuator; wherein the injection needle subassembly comprises a first telescoping guide needle having an inner cannula and an outer diameter; and a second cannula having a second inner cannula slidably engaged with the outer diameter of the first telescoping guide needle; a delivery catheter/injection needle inserted through the first inner and second inner cannulas and connecting at one end with the pre-filled syringe needle connector and formed into a needle point at the opposite end; wherein the delivery catheter is secured to the interior surface of the second cannula; and wherein the second cannula is connected to the at least one linear actuator; c) a macro-positioning subassembly for orienting the delivery catheter in the x, y and z axes relative to an animal or human positioned adjacent the automated injection device; further comprising a goniometer comprising a macro-angular adjustment and/or a micro-angular adjustment; and d) a programmable controller capable of controlling the at least one linear actuator to (i) advance and retract the injection needle and (ii) to control the volume and flow rate of the contents of the pre-filled syringe through actuation of the plunger rod in the operation of the automated injection device.

Embodiment 3: An injection device for delivering a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) at least one linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by the at least one linear actuator; wherein the injection needle subassembly comprises a first telescoping guide needle having an inner cannula and an outer diameter; and a second cannula having a second inner cannula slidably engaged with the outer diameter of the first telescoping guide needle; a delivery catheter/injection needle inserted through the first inner and second inner cannulas and connecting at one end with the pre-filled syringe needle connector and formed into a needle point at the opposite end; wherein the delivery catheter is secured to the interior surface of the second cannula; and wherein the second cannula is connected to the at least one linear actuator; c) a macro-positioning subassembly for orienting the delivery catheter in the x, y and z axes relative to an animal or human positioned adjacent the automated injection device; further comprising a goniometer comprising a macro-angular adjustment; a vertical height adjustable post, an adjustable articulated arm; and d) a programmable controller capable of controlling the at least one linear actuator to (i) advance and retract the injection needle and (ii) to control the volume and flow rate of the contents of the pre-filled syringe through actuation of the plunger rod in the operation of the automated injection device.

Embodiment 4: An injection device for delivering a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) at least one linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by the at least linear actuator; wherein the injection needle subassembly comprises a first telescoping guide tool/needle having an inner cannula and an outer diameter; and a second rigid cannula having a second inner cannula slidably engaged with the outer diameter of the first telescoping guide needle; a delivery catheter/injection needle inserted through the first inner and second inner cannulas and connecting at one end with the pre-filled syringe needle connector and formed into a needle point at the opposite end; wherein the delivery catheter is secured to the interior surface of the second cannula; and wherein the second cannula is connected to the at least one linear actuator; c) a macro-positioning subassembly for orienting the flexible wire catheter in the x, y and z axes relative to an animal or human positioned adjacent the automated injection device; further comprising a goniometer comprising a macro-angular adjustment; a vertical height adjustable post, an adjustable articulated arm and a micro-angular adjustment; wherein the micro-positioning subassembly further comprises: a first horizontal support arm; a second horizontal support arm oriented at right angles to the first horizontal support arm; and a rotatable stage member; wherein the first horizontal support arm comprises one or more adjustable vertical support rail attached to a first vertical support rail micro-adjustor for adjusting the first horizontal support arm along the z axis; further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more rails attached at the top of the goniometer rail to the bottom surface of the rotatable stage; and d) a programmable controller capable of controlling the at least one linear actuator to (i) advance and retract the injection needle and (ii) to control the volume and flow rate of the contents of the pre-filled syringe through actuation of the plunger rod in the operation of the automated injection system.

Embodiment 5: An injection device for delivering a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) at least one linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable; wherein the syringe comprises a needle connector at one end and a plunger attached to a plunger rod at the opposite end; wherein the plunger rod is actuated by the at least linear actuator; wherein the injection needle subassembly comprises (i) a delivery catheter comprising a syringe needle connector capable of attaching to the needle connector of the pre-filled syringe at one end and having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide needle; wherein the delivery catheter/injection needle is dimensioned to pass through the telescoping two-part slide mechanism; further wherein the flexible wire catheter is secured to the interior of the outer cannula thereby providing for vertical movement of the outer cannula and attached delivery catheter upon actuation of the at least one linear actuator; and further wherein the delivery catheter is capable of forming a service loop at the end of the catheter attached to the prefilled syringe; further wherein the outer cannula is attached to a first mounting block that connects to the first linear actuator connector between the injection needle subassembly and the linear actuator; and wherein the inner cannula is attached to a second mounting block that rigidly connects to the injection needle subassembly connector of the injector device subassembly; and c) a macro-positioning subassembly for orienting the flexible wire catheter in the x, y and z axes relative to an animal or human positioned adjacent the automated injection device; further a vertical height adjustable post, an adjustable articulated arm; and d) a programmable controller capable of controlling the at least one linear actuator to (i) advance and retract the delivery catheter/injection needle and (ii) to control the volume and flow rate of the contents of the pre-filled syringe through actuation of the plunger rod in the operation of the automated injection device.

Embodiment 6: An injection device for delivering trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) at least one linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by the at least linear actuator; wherein the injection needle subassembly comprises (i) a delivery catheter comprising a syringe needle connector capable of attaching to the needle connector of the pre-filled syringe at one end and having a needle point at the other end of the delivery catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide tube/needle; wherein the delivery catheter/injection needle is dimensioned to pass through the telescoping two-part slide mechanism; further wherein the flexible wire catheter is secured to the interior of the outer cannula thereby providing for vertical movement of the outer cannula and attached flexible wire catheter upon actuation of the at least one linear actuator; and further wherein the flexible metallic catheter is capable of forming an injection needle service loop at the end of the catheter attached to the prefilled syringe; further wherein the outer cannula is attached to a first mounting block that connects to the at least one linear actuator connector between the injection needle subassembly and the at least one linear actuator; and wherein the inner cannula is attached to a second mounting block that rigidly connects to the injection needle subassembly connector of the injector device subassembly; and c) a macro-positioning subassembly for orienting the flexible wire catheter in the x, y and z axes relative to a prone animal or human positioned under the automated injection device; further comprising a goniometer comprising a macro-angular adjustment; a vertical height adjustable post, an adjustable articulated arm; and d) a programmable controller capable of controlling the at least one linear actuator to (i) advance and retract the injection needle and (ii) to control the volume and flow rate of the contents of the pre-filled syringe through actuation of the plunger rod in the operation of the automated injection device.

Embodiment 7: An injection device for delivering trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) at least one linear actuator; b) an injector device sub-assembly for actuating (1) a separately provided injection needle subassembly and a (2) a separately provided pre-filled syringe containing cells and/or a therapeutic substance; wherein the syringe comprises a needle connector at one end and a plunger attached to a plunger rod at the opposite end; wherein the plunger rod is actuated by the at least linear actuator; wherein the injection needle subassembly comprises (i) a flexible metallic catheter comprising a syringe needle connector capable of attaching to the needle connector of the pre-filled syringe at one end and having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide needle; wherein the delivery catheter/injection needle is dimensioned to pass through the telescoping two-part slide mechanism; further wherein the delivery catheter is secured to the interior of the outer cannula thereby providing for vertical movement of the outer cannula and attached delivery catheter upon actuation of the at least one linear actuator; and further wherein the delivery catheter is capable of forming a service loop at the end of the delivery catheter attached to the prefilled syringe; further wherein the outer cannula is attached to a first mounting block that connects to the at least one linear actuator connector between the injection needle subassembly and the at least one linear actuator; and wherein the inner cannula is attached to a second mounting block that rigidly connects to the injection needle subassembly connector of the injector device subassembly; and c) a macro-positioning subassembly for orienting the delivery catheter in the x, y and z axes relative to an animal or human positioned adjacent the automated injection device; further comprising a goniometer comprising a macro-angular adjustment; a vertical height adjustable post, an adjustable articulated arm; further comprising a vertical height adjustable post, an adjustable articulated arm and a micro-positioning subassembly; wherein the micro-positioning subassembly further comprises: a first horizontal support arm; a second horizontal support arm oriented at right angles to the first horizontal support arm; and a rotatable stage member; wherein the first horizontal support arm comprises one or more adjustable vertical support rail attached to a first vertical support rail micro-adjustor for adjusting the first horizontal support arm along the z axis; and further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more rails attached at the top of the goniometer rail to the bottom surface of the rotatable stage; and d) a programmable controller capable of controlling the at least one linear actuator to (i) advance and retract the injection needle and (ii) to control the volume and flow rate of the contents of the pre-filled syringe through actuation of the plunger rod in the operation of the automated injection device.

Embodiment 8: An injection device for delivering a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: (1) an injection needle subassembly; (2) a separately provided prefilled syringe comprising an injection needle connector at one end and a plunger connected to a plunger rod; (3) at least one linear actuator; (4) one or more injector device subassembly mounting connectors; (5) an injection needle subassembly connector; (6) a first linear actuator connector between the injection needle subassembly and the linear actuator; and (7) a second linear actuator connector between the plunger rod and the linear actuator, wherein the second linear actuator connector is capable of controlling the volume and flow rate of the pre-filled syringe by actuation of the plunger rod in the operation of the injection device; b) a macro-positioning sub-assembly for roughly adjusting the orientation of the automated injector device sub-assembly along x, y and z axes relative to an animal or human positioned adjacent the automated injection device, comprising a vertical height adjustable post, an adjustable articulated arm, and a micro-positioning subassembly; wherein the micro-positioning subassembly further comprises: a first horizontal support arm; a second horizontal support arm oriented at right angles to the first horizontal support arm; a rotatable stage member; and a goniometer comprising goniometer a macro-angular adjustment and a goniometer micro-angular adjustment; wherein the first horizontal support arm comprises one or more adjustable vertical support rail attached to a first vertical support rail micro-adjustor for adjusting the first horizontal support arm along the z axis; and further wherein the first horizontal support arm further comprises a first horizontal rail attached to a first horizontal rail micro- adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more second adjustable goniometer rail attached at the top of the goniometer rail to the bottom surface of the rotatable stage; c) further comprising a separately provided injection needle subassembly, wherein the injection needle subassembly comprises: (i) a delivery catheter comprising a syringe needle connector capable of attaching to the needle connector of the pre-filled syringe at one end and having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide tube/needle; wherein the delivery catheter/injection needle is dimensioned to pass through the telescoping two-part slide mechanism; further wherein the delivery catheter is secured to the interior of the outer cannula thereby providing for vertical movement of the outer cannula and attached delivery catheter upon actuation of the at least one linear actuator; and further wherein the delivery catheter is capable of forming a service loop at the end of the catheter attached to the prefilled syringe; further wherein the outer cannula is attached to a first mounting block that connects to the at least one linear actuator connector between the injection needle subassembly and the at least one linear actuator; and wherein the inner cannula is attached to a second mounting block that rigidly connects to the injection needle subassembly connector of the injector device subassembly; and d) a programmable controller capable of controlling volume and flow rate of the pre-filled syringe in operation.

Embodiment 9: An injection device for delivering a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) a first and a second linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by the second linear actuator; wherein the injection needle subassembly comprises a first telescoping guide tube/needle having an inner cannula and an outer diameter; and a second cannula having a second inner cannula slidably engaged with the outer diameter of the first telescoping guide tube/needle; a delivery catheter/injection needle inserted through the first inner and second inner cannulas and connecting at one end with the pre-filled syringe needle connector and formed into a needle point at the opposite end; wherein the delivery catheter is secured to the interior surface of the second rigid cannula; and wherein the second rigid cannula and the plunger rod are connected to the first linear actuator; c) a macro-positioning subassembly for orienting the flexible wire catheter in the x, y and z axes relative to a prone animal or human positioned under the injection device; and d) a programmable controller capable of controlling the at first and second linear actuators to (i) advance and retract the injection needle and (ii) to control the volume and flow rate of the contents of the pre-filled syringe through actuation of the plunger rod in the operation of the injection system.

Embodiment 10: An injection device for delivering trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) a first and second linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by the second linear actuator; wherein the injection needle subassembly comprises a first telescoping guide tube/needle having an inner cannula and an outer diameter; and a second rigid cannula having a second inner cannula slidably engaged with the outer diameter of the first telescoping guide needle; a delivery catheter injection needle inserted through the first inner and second inner cannulas and connecting at one end with the pre-filled syringe needle connector and formed into a needle point at the opposite end; wherein the delivery catheter is secured to the interior surface of the second cannula; and wherein the second cannula is connected to the first linear actuator; c) a macro-positioning subassembly for orienting the flexible wire catheter in the x, y and z axes relative to a prone animal or human positioned under the automated injection device; further comprising a goniometer comprising a macro-angular adjustment and/or a micro- angular adjustment; and d) a programmable controller capable of controlling the first and second linear actuators to (i) advance and retract the delivery catheter/injection needle and (ii) to control the volume and flow rate of the contents of the pre-filled syringe through actuation of the plunger rod in the operation of the automated injection system.

Embodiment 11: An injection device for delivering trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) a first and second linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable; wherein the syringe comprises a needle connector at one end and a plunger attached to a plunger rod at the opposite end; wherein the plunger rod is actuated by the second linear actuator; wherein the injection needle subassembly comprises a first telescoping guide needle having an inner cannula and an outer diameter; and a second rigid cannula having a second inner cannula slidably engaged with the outer diameter of the first telescoping guide needle; a delivery catheter/injection needle inserted through the first inner and second inner cannulas and connecting at one end with the pre-filled syringe needle connector and formed into a needle point at the opposite end; wherein the flexible wire catheter is secured to the interior surface of the second rigid cannula; and wherein the second rigid cannula is connected to the first linear actuator; c) a macro-positioning subassembly for orienting the flexible wire catheter in the x, y and z axes relative to a prone animal or human positioned under the automated injection device; further comprising a goniometer comprising a macro-angular adjustment; a vertical height adjustable post, an adjustable articulated arm; and d) a programmable controller capable of controlling the first and second linear actuators to (i) advance and retract the injection needle and (ii) to control the volume and flow rate of the contents of the pre-filled syringe through actuation of the plunger rod in the operation of the automated injection device.

Embodiment 12: An injection device for delivering a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) a first and second linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by the second linear actuator; wherein the injection needle subassembly comprises a first telescoping guide needle having an inner cannula and an outer diameter; and a second rigid cannula having a second inner cannula slidably engaged with the outer diameter of the first telescoping guide needle; a delivery needle inserted through the first inner and second inner cannulas and connecting at one end with the pre-filled syringe needle connector and formed into a needle point at the opposite end; wherein the delivery catheter is secured to the interior surface of the second rigid cannula; and wherein the second rigid cannula is connected to the first linear actuator; c) a macro-positioning subassembly for orienting the delivery catheter in the x, y and z axes relative to a prone animal or human positioned under the automated injection device; further comprising a goniometer comprising a macro-angular adjustment; a vertical height adjustable post, an adjustable articulated arm and a micro-angular adjustment; wherein the micro-positioning subassembly further comprises: a first horizontal support arm; a second horizontal support arm oriented at right angles to the first horizontal support arm; and a rotatable stage member; wherein the first horizontal support arm comprises one or more adjustable vertical support rail attached to a first vertical support rail micro-adjustor for adjusting the first horizontal support arm along the z axis; further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more rails attached at the top of the goniometer rail to the bottom surface of the rotatable stage; and d) a programmable controller capable of controlling the first and second linear actuators to (i) advance and retract the injection needle and (ii) to control the volume and flow rate of the contents of the pre-filled syringe through actuation of the plunger rod in the operation of the automated injection system.

Embodiment 13: An injection device for delivering trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) a first and second linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by the second linear actuator; wherein the injection needle subassembly comprises (i) a delivery catheter comprising a syringe needle connector capable of attaching to the needle connector of the pre-filled syringe at one end and having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide needle; wherein the delivery catheter/injection needle is dimensioned to pass through the telescoping two-part slide mechanism; further wherein the flexible wire catheter is secured to the interior of the outer cannula thereby providing for vertical movement of the outer cannula and attached flexible wire catheter upon actuation of the first linear actuator; and further wherein the flexible metallic catheter is capable of forming an injection needle service loop at the end of the catheter attached to the prefilled syringe; further wherein the outer cannula is attached to a first mounting block that connects to the first linear actuator connector between the injection needle subassembly and the first linear actuator; and wherein the inner cannula is attached to a second mounting block that rigidly connects to the injection needle subassembly connector of the injector device subassembly; and c) a macro-positioning subassembly for orienting the flexible wire catheter in the x, y and z axes relative to a prone animal or human positioned under the automated injection device; further a vertical height adjustable post, an adjustable articulated arm; and d) a programmable controller capable of controlling the first and second linear actuators to (i) advance and retract the injection needle and (ii) to control the volume and flow rate of the contents of the pre-filled syringe through actuation of the plunger rod in the operation of the automated injection system.

Embodiment 14: An injection device for delivering a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) a first and second linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by second linear actuator; wherein the injection needle subassembly comprises (i) a delivery catheter comprising a syringe needle connector capable of attaching to the needle connector of the pre-filled syringe at one end and having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide needle; wherein the delivery catheter/injection needle is dimensioned to pass through the telescoping two-part slide mechanism; further wherein the delivery catheter is secured to the interior of the outer cannula thereby providing for vertical movement of the outer cannula and attached flexible wire catheter upon actuation of the first linear actuator; and further wherein the delivery catheter is capable of forming an service loop at the end of the catheter attached to the prefilled syringe; further wherein the outer cannula is attached to a first mounting block that connects to the first linear actuator connector between the injection needle subassembly and the first linear actuator; and wherein the inner cannula is attached to a second mounting block that rigidly connects to the injection needle subassembly connector of the injector device subassembly; and c) a macro-positioning subassembly for orienting the flexible wire catheter in the x, y and z axes relative to a prone animal or human positioned under the automated injection device; further comprising a goniometer comprising a macro-angular adjustment; a vertical height adjustable post, an adjustable articulated arm; and d) a programmable controller capable of controlling the first and second linear actuators to (i) advance and retract the injection needle and (ii) to control the volume and flow rate of the contents of the pre-filled syringe through actuation of the plunger rod in the operation of the automated injection device. In certain additional embodiments, the needle point may be fabricated as a blunt or curved tip.

Embodiment 15: An injection device for delivering trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) a first and second linear actuator; b) an injector device sub-assembly for actuating (1) a separately provided injection needle subassembly and a (2) a separately provided pre-filled syringe containing therapeutic cells and/or one or more therapeutic substances or diagnostic substances or injectable medium; wherein the syringe comprises a needle connector at one end and a plunger attached to a plunger rod at the opposite end; wherein the plunger rod is actuated by the second linear actuator; wherein the injection needle subassembly comprises (i) a delivery catheter comprising a syringe needle connector capable of attaching to the needle connector of the pre-filled syringe at one end and having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide needle; wherein the delivery catheter/injection needle is dimensioned to pass through the telescoping two-part slide mechanism; further wherein the delivery catheter is secured to the interior of the outer cannula thereby providing for vertical movement of the outer cannula and attached flexible wire catheter upon actuation of the first linear actuator; and further wherein the flexible metallic catheter is capable of forming an service loop at the end of the catheter attached to the prefilled syringe; further wherein the outer cannula is attached to a first mounting block that connects to the first linear actuator connector between the injection needle subassembly and the first linear actuator; and wherein the inner cannula is attached to a second mounting block that rigidly connects to the injection needle subassembly connector of the injector device subassembly; and c) a macro-positioning subassembly for orienting the flexible wire catheter in the x, y and z axes relative to a prone animal or human positioned under the automated injection device; further comprising a goniometer comprising a macro-angular adjustment; a vertical height adjustable post, an adjustable articulated arm; further comprising a vertical height adjustable post, an adjustable articulated arm and a micro-positioning subassembly; wherein the micro-positioning subassembly further comprises: a first horizontal support arm; a second horizontal support arm oriented at right angles to the first horizontal support arm; and a rotatable stage member; wherein the first horizontal support arm comprises one or more adjustable vertical support rail attached to a first vertical support rail micro-adjustor for adjusting the first horizontal support arm along the z axis; and further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more rails attached at the top of the goniometer rail to the bottom surface of the rotatable stage; and d) a programmable controller capable of controlling the first and second linear actuators to (i) advance and retract the delivery catheter/injection needle and (ii) to control the volume and flow rate of the contents of the pre-filled syringe through actuation of the plunger rod in the operation of the automated injection device.

Embodiment 16: An injection system for delivering a trail of therapeutic cells and/or one or more therapeutic substances or diagnostic substances or injectable medium into an anatomical space of an animal or human subject, particularly a trail of therapeutic cells and/or one or more therapeutic substances or diagnostic substances or injectable medium into the spinal cord of a subject and to deliver a trail of therapeutic cells and/or one or more therapeutic substances or diagnostic substances or injectable medium inside the spinal cord, comprising: a) an injector device subassembly comprising: (1) an injection needle subassembly; (2) a separately provided prefilled syringe comprising an injection needle connector at one end and a plunger connected to a plunger rod; (3) a first and second linear actuator; (4) one or more injector device subassembly mounting connectors; (5) an injection needle subassembly connector; (6) a first linear actuator connector between the injection needle subassembly and the first linear actuator; and (7) a second linear actuator connector between the plunger rod and the second linear actuator, wherein the second linear actuator connector is capable of controlling the volume and flow rate of the pre-filled syringe by actuation of the plunger rod in the operation of the injection system; b) a macro-positioning sub-assembly for roughly adjusting the orientation of the automated injector device sub-assembly along x, y and z axes relative to an animal or human positioned adjacent the automated injection device, comprising a vertical height adjustable post, an adjustable articulated arm, and a micro-positioning subassembly; wherein the micro-positioning subassembly further comprises: a first horizontal support arm; a second horizontal support arm oriented at right angles to the first horizontal support arm; a rotatable stage member; and a goniometer comprising goniometer a macro-angular adjustment and a goniometer micro-angular adjustment; wherein the first horizontal support arm comprises one or more adjustable vertical support rail attached to a first vertical support rail micro-adjustor for adjusting the first horizontal support arm along the z axis; and further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more second adjustable goniometer rail attached at the top of the goniometer rail to the bottom surface of the rotatable stage; c) further comprising a separately provided injection needle subassembly, wherein the injection needle subassembly comprises: (i) a delivery catheter comprising a syringe needle connector capable of attaching to the needle connector of the pre-filled syringe at one end and having a needle point at the other end of the catheter; (ii) a telescoping two-part slide mechanism comprising: (x) an outer cylindrical cannula and (y) an inner cannula; wherein the inner cannula is dimensioned at one end to slide snugly without excessive friction within the outer cannula, further wherein the inner cannula is bent at the opposite end into a guide needle; wherein the delivery catheter/injection needle is dimensioned to pass through the telescoping two-part slide mechanism; further wherein the flexible wire catheter is secured to the interior of the outer cannula thereby providing for vertical movement of the outer cannula and attached flexible wire catheter upon actuation of the first linear actuator; and further wherein the flexible metallic catheter is capable of forming an injection needle service loop at the end of the catheter attached to the prefilled syringe; further wherein the outer cannula is attached to a first mounting block that connects to the first linear actuator connector between the injection needle subassembly and the first linear actuator; and wherein the inner cannula is attached to a second mounting block that rigidly connects to the injection needle subassembly connector of the injector device subassembly; and d) a programmable controller capable of controlling volume and flow rate of the pre-filled syringe in operation.

Embodiment 17: Embodiments 1-16, wherein the guide needle is bent.

Embodiment 18: Embodiment 1-16, wherein the bend angle of the guide needle is about 100°.

Embodiment 19: Embodiments 1-16, wherein the pre-filled syringe needle connector is a Hamilton removable needle connection or a Luer connector.

Embodiment 20: Embodiments 1-16, wherein the delivery needle is manufactured from a nickel-titanium alloy.

Embodiment 21: Embodiment 20, wherein the nickel-titanium alloy has an oxide finish.

Embodiment 22: Embodiments 1-16, wherein the delivery needle is 29 gauge.

Embodiment 23: Embodiments 1-16, wherein the delivery needle is secured with an epoxy adhesive.

Embodiment 24: Embodiments 1-16 further comprising a mobile cart for supporting the injection device axes relative to a prone animal or human positioned under the injection device.

Embodiment 25: Embodiments 1-16 further comprising a macro height adjustment actuating the vertical height adjustable post.

Embodiment 26: Embodiments 1-16, wherein the macro-positioning subassembly attached to a surgical table or a hospital bed.

Embodiment 27: In another aspect of the invention, a system for delivering a trail of therapeutic cells and/or one or more therapeutic substances or diagnostic substances or injectable medium into a spinal cord is described, the system comprising: a delivery catheter configured to enter the spinal cord and deliver therapeutic cells and/or one or more therapeutic substances or diagnostic substances or injectable medium inside the spinal cord; an guide tube/introducer needle having a proximal end and a distal end, wherein: the introducer needle houses the delivery catheter, and the guide tube/introducer needle is configured to introduce the delivery catheter into the spinal cord through the distal end; a linear actuator located near the proximal end of the guide tube/introducer needle and configured to move the delivery catheter inside the guide tube/introducer needle; and a second guide tubing located between the linear actuator and the proximal end of the introducer needle, wherein the second guide tubing houses and guides a portion of the delivery catheter between the linear actuator and the proximal end of the introducer needle.

Embodiment 28: In another embodiment of the present invention, a method of injecting a trail of therapeutic cells and/or one or more therapeutic substances or diagnostic substances or injectable medium directly into the central nervous system, in particular, directly into the spinal cord parenchyma, employing the injection apparatus of any one of the foregoing aspects of the present invention.

Embodiment 29: In additional embodiments, the delivery catheter/needle may be a flexible catheter.

FIG. 18 shows an embodiment of a complete therapeutic trail injection system 900 comprising an optional mobile support cart 901 having legs 902 and table top 903 for supporting a macro or vertical adjustable post 904, comprising an adjustable vertical post 904, a height adjustment mechanism 905 comprising an adjustment wheel 906 and knob 907, as well as a horizontal selective compliance articulated robot arm (SCARA) positioning arm 910 having one or more adjustment knobs 912. SCARA positioning arm supports the XYZ mounting system 915 for injection dispensing device 940. XYZ mounting system 915 comprises: a horizontal support member 920, having one or more micro adjustment wheels 921; a vertical support member 925, having micro adjustment wheel 976; rotatable platform 930, having one or more locking screws 933 and micromanipulator 931. Injection dispensing device 940 is attached to rotatable platform 930. Also shown positioned on table top 903 is control panel 990. Mobile cart 901 and injection device 900 are configured to permit positioning injection device 900 adjacent to a surgical bed (not shown) through adjustments along three axes (x, y and z) (not shown).

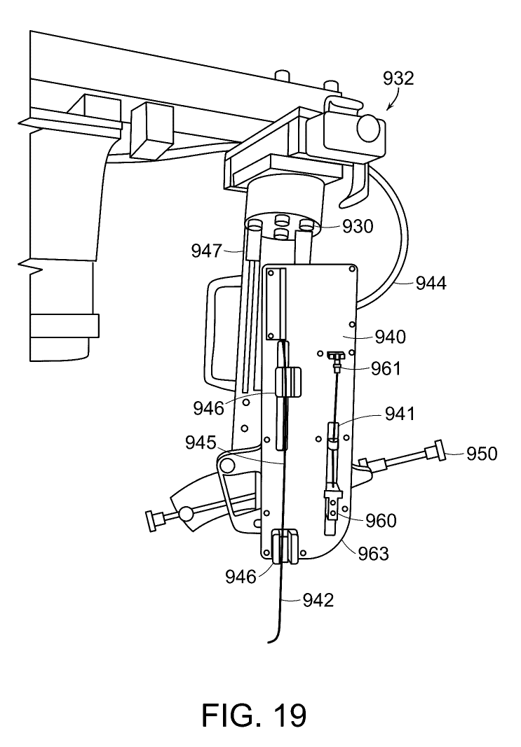

FIG. 19 shows a view of the injection dispensing device 940 suspended by one or more arms 947 from rotatable platform 930, controlled by one or more micromanipulators 932 and bearing a syringe 941 containing therapeutic cells and/or one or more therapeutic substances or diagnostic substances or injectable medium and a guide tube/needle 942 housing beveled delivery catheter/injection needle 943 (not shown) and an adjustable goniometer for pitch adjustment 950. The injection dispensing device also comprises a motorized syringe mechanism 960, a motorized injection needle 943 actuated by a linear actuator (not shown) terminating in beveled injection needle at the distal end thereof in some embodiments (not shown). In other embodiments, the distal end of delivery catheter/injection needled 943 may be blunt, curved or shaped in some other geometry. Motorized injection needle 943 forms service loop 944 through a snap-on connector 946 attached to trombone mechanism 945a and 945b (not completely shown) and a second snap-on connector 946 attached to guide needle 942. Motorized injection needle 943 runs through injection service loop 944, trombone mechanism 945 and guide needle 942 before emerging and penetrating or running along the surface of the spinal cord or beneath the pia matter of a subject (not shown). Syringe 941 is supported by a syringe clip attachment 948 (not shown) at each end and the plunger rod of syringe 941 is attached to motorized plunger drive 963 (shown in part).

FIG. 20 shows syringe 941 in more detail having a connection fitting such as a luer-lock fitting 941a and plunger 941b (not shown) and plunger rod 941c. Syringe connection 941a connects with syringe connector 949 attached to one end of service loop 944, and then through a trombone mechanism 945 comprising outer trombone barrel 945a and inner trombone barrel 945b supported at each end by snap-on connections 946 and terminating in a beveled, blunt or other configuration distal end of injection needle 943 (not shown) passing through guide tube/needle 942. It is to be noted that in some embodiments such as depicted in FIG. 20, guide tube 942 comprises trombone assembly 945a and 945b and service loop 944, terminating at the proximal end thereof in syringe connector 949. The distal end of guide tube 942 (i.e. lower trombone tube 945b may terminate in a needle point, a blunt end, a curved end or in some other configuration in different embodiments.

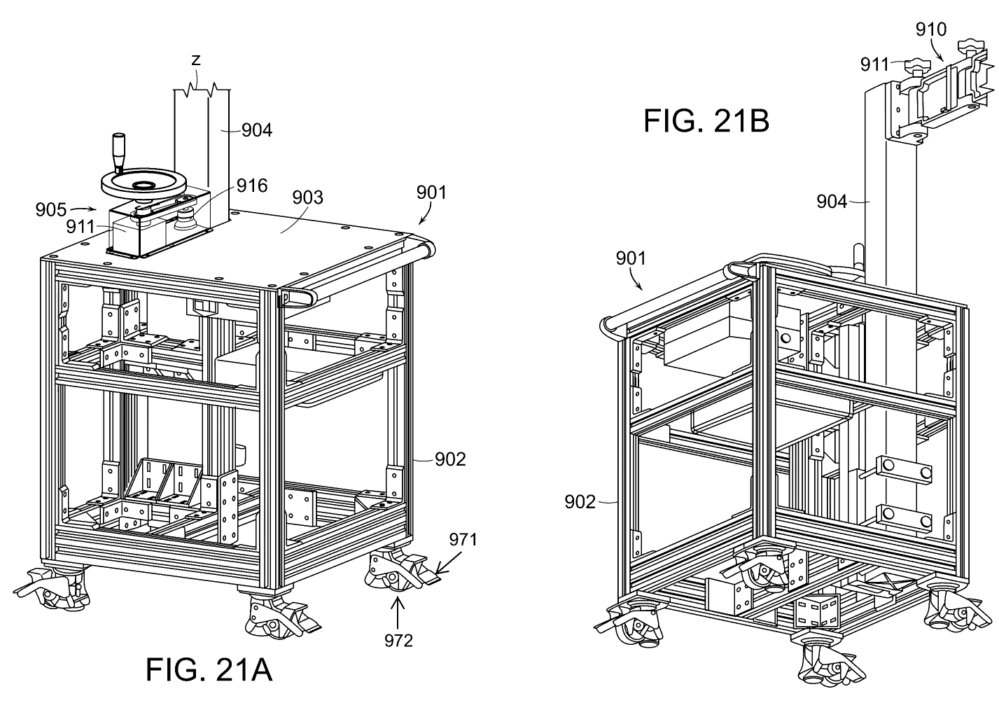

FIGS. 21A and 21B show a more detailed view of mobile cart 901 from different perspectives. Mobile cart 901 comprises a mobile height adjustment 905 shown on FIG. 21A and vertical macro height post 904. In an embodiment, support legs 902 support table top 903 and allow the mobile cart 901 to be secured to the floor by virtue of a plurality of locking wheel mechanisms 971 on wheels 972. The injection system comprises a macro height adjustment 905 that controls macro height post 904 by virtue of a gearing arrangement 911 and 916 and a chain (not shown). Also as shown in FIG. 21A, the macro height adjustment 905 comprises a gearing mechanism (not shown) as known in the art, and macro height post 904 allows adjustment of injection device 900 (not shown) in a vertical direction along the z-axis (FIG. 21A). This permits the injection system 900 to be positioned over the patient. Macro height post 904 is supported by a plurality of brackets 973. Macro height post supports a selective compliance articulated robot arm (SCARA arm) 910, as shown in FIG. 21B.

FIG. 22A and 22B show, respectively, different views of macro height post 904 at different heights, as controlled by macro height mechanism 905 affixed to mobile cart 901, in both use (FIG. 22A) and rest positions (FIG. 22B). Also shown on mobile cart 901 is controller 990 and SCARA positioning arm 910. The macro height post 904 and macro height adjustment 905 permit vertical adjustment of the trail injection device 900 in the operation of an embodiment. The macro height post 904 supporting a SCARA arm 910 permits three dimensional adjustments in the x, y and z axes in association with XYZ mounting system 915. The three dimensional control of the injection needle device 930 (partially shown) enables the surgeon or surgical assistant to control the three dimensional positioning of the guide tube 942 (housing injection needle 943) for penetration of the spinal cord of a subject (not shown).

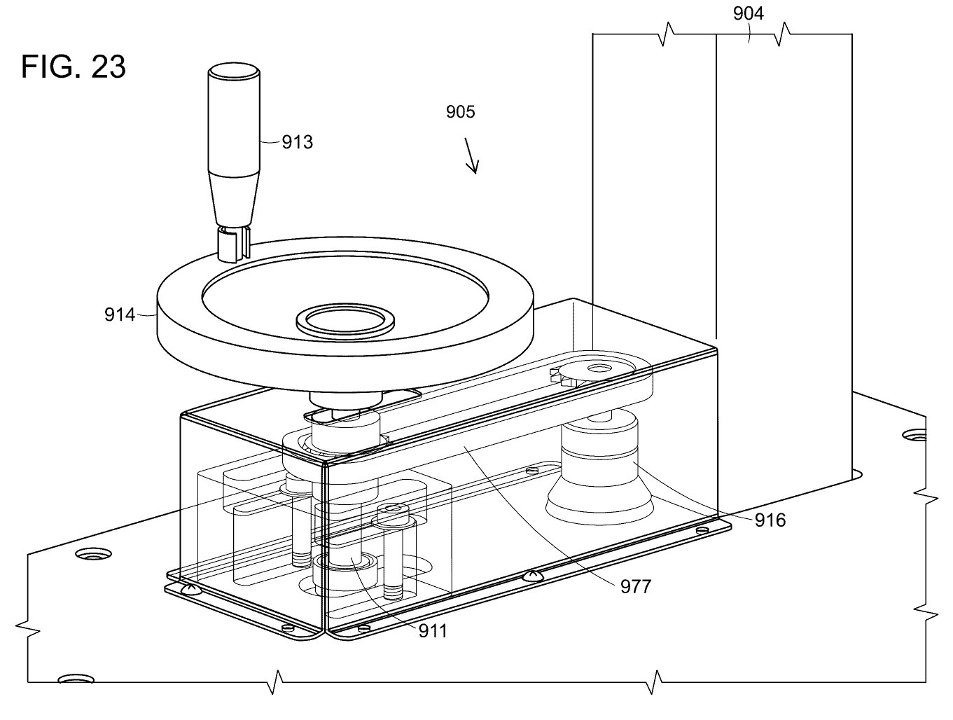

FIG. 23 is an enlarged graphical representation of a macro height adjustment mechanism 905 that controls a macro height post 904 by virtue of a gearing drives 911 and 916 and a chain, band, or belt 977 or like connecting drives 911 and 916. The vertical extension of the macro height post 904 permits the trail injection device 900 (not shown) to be positioned over the patient and to control height adjustments in the “z” axis.

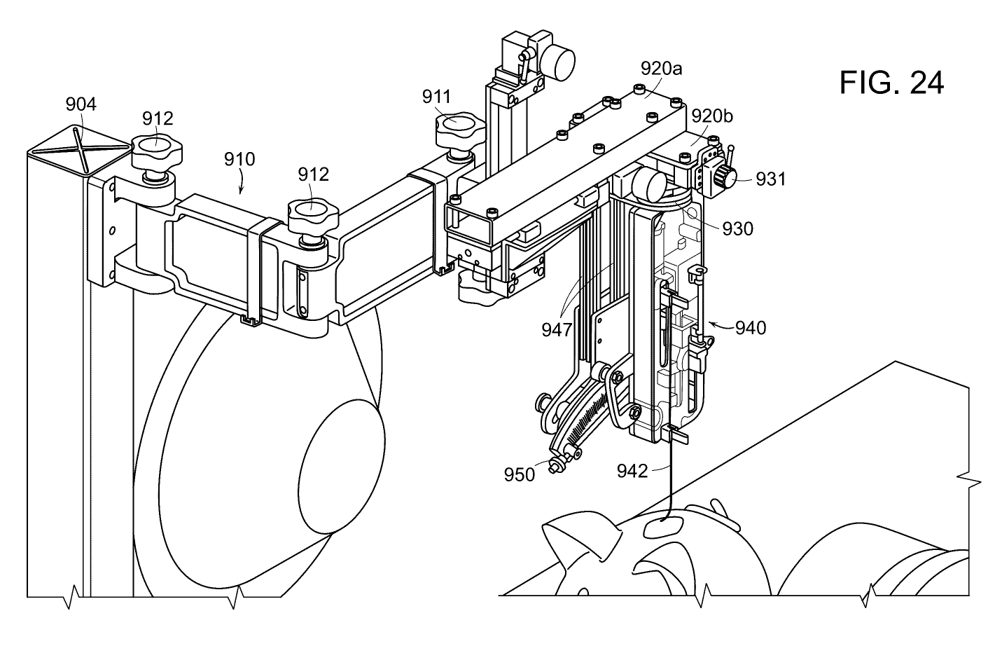

FIG. 24 an enlarged graphical representation of a SCARA positioning arm 910 in use showing macro adjustment wheels 912 for adjusting the direction of the SCARA positioning arm 910 in the “x” and “y” directions , linear rails 947, rotating platform 930, injection dispensing device 940 and goniometer adjustment 950 (partial view) as well as macro height post 904. Macro height post 904, controlled by macro height adjustment 905 (not shown) allows the post to be raised and lowered to allow the SCARA positioning arm and injection dispensing device to be positioned over the subject. In an embodiment, SCARA positioning arm 910 has at the end opposite the macro height adjustment post 904 horizontal supports 920a and 920b are used to adjust the injection dispensing device in the “x” and “y” directions. Rotation stage 930 is positioned with its center of the tip of guide needle 942 (not shown) allowing the guide needle to be rotated 360 degrees about its axis. Rotation of the rotation stage 930 revolves the guide tube 942 around its distal tip (thereby orienting the distal tip of the delivery catheter/injection needle). Goniometer 950 permits pitch adjustment of the guide needle 942. Thus, goniometer 950 tilts the guide tube/needle around its tip, allowing for precise angling of the injection needle 943 (not shown) into the spinal cord. In an embodiment, guide needle 942 can be adjusted +/−30 degrees without changing the design of the guide needle.

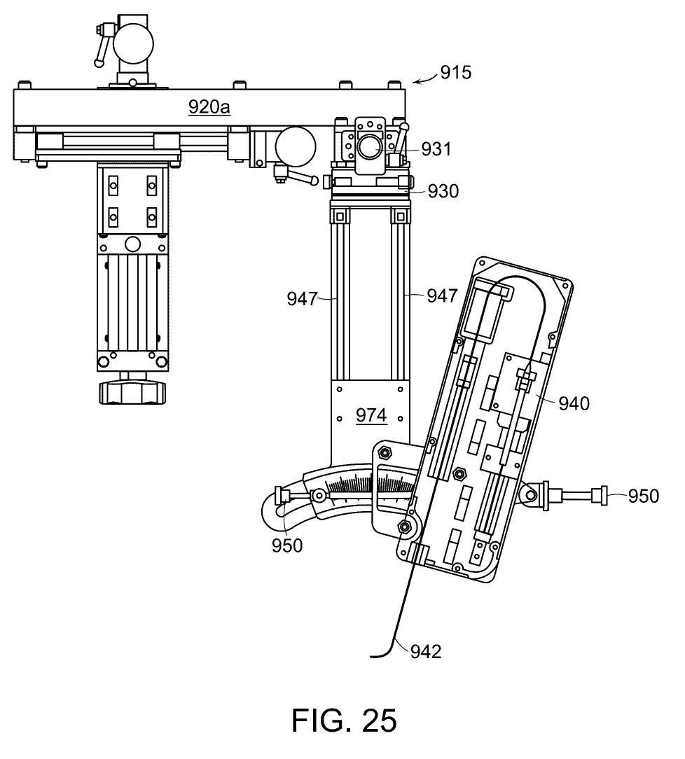

FIG. 25 is a graphical representation showing XYZ positioning system 915 comprising horizontal arms 920a and 920b (not shown), rotating platform 930 and y adjustment 931, positioning rails 947 for injection dispensing device 940 attached by bracket 974 and adjustment of the angle of guide needle 942 by adjusting the angle by altering the goniometer settings of goniometer 950.

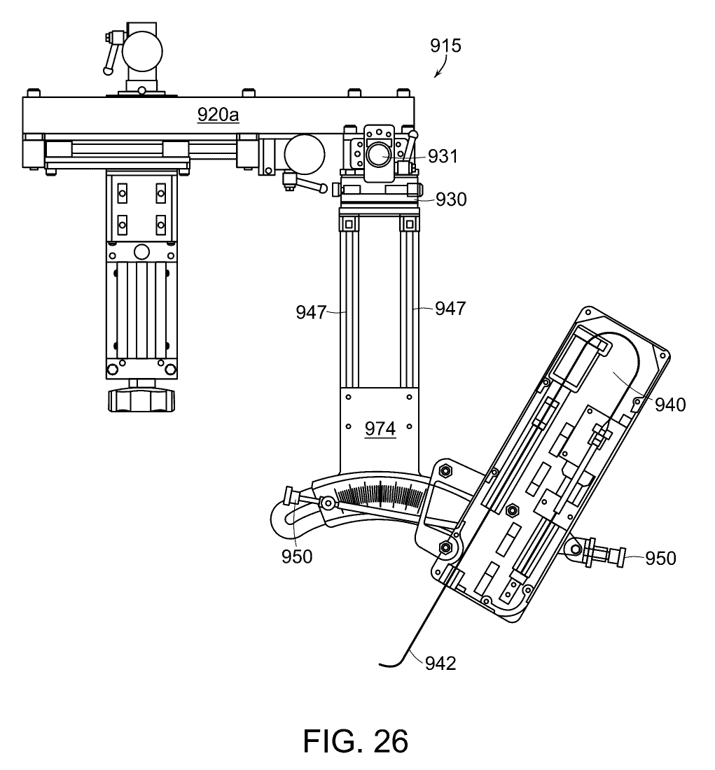

FIG. 26 is a graphical representation showing a different adjustment of the angle of guide needle 942 by adjusting the angle of the goniometer 950 settings compared to FIG. 25.

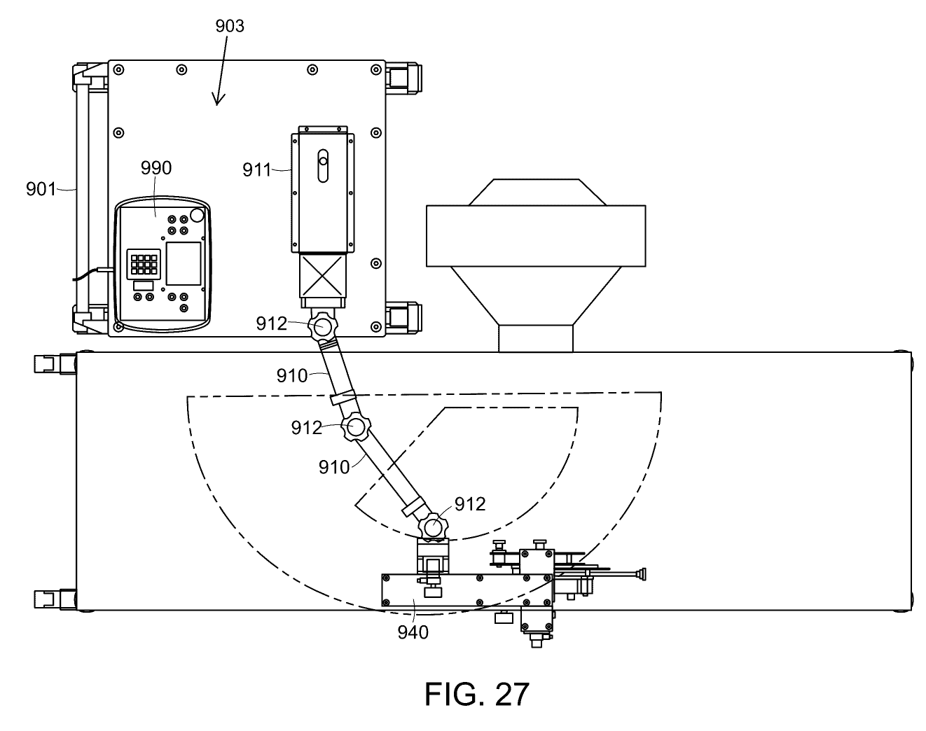

FIG. 27 is a graphical representation of the operation and adjustment of the SCARA positioning arm 910 through adjustment of SCARA arm adjusters 912 to permit orientation of the injection dispensing device and guide needle along the “x” and “y” directions in use.

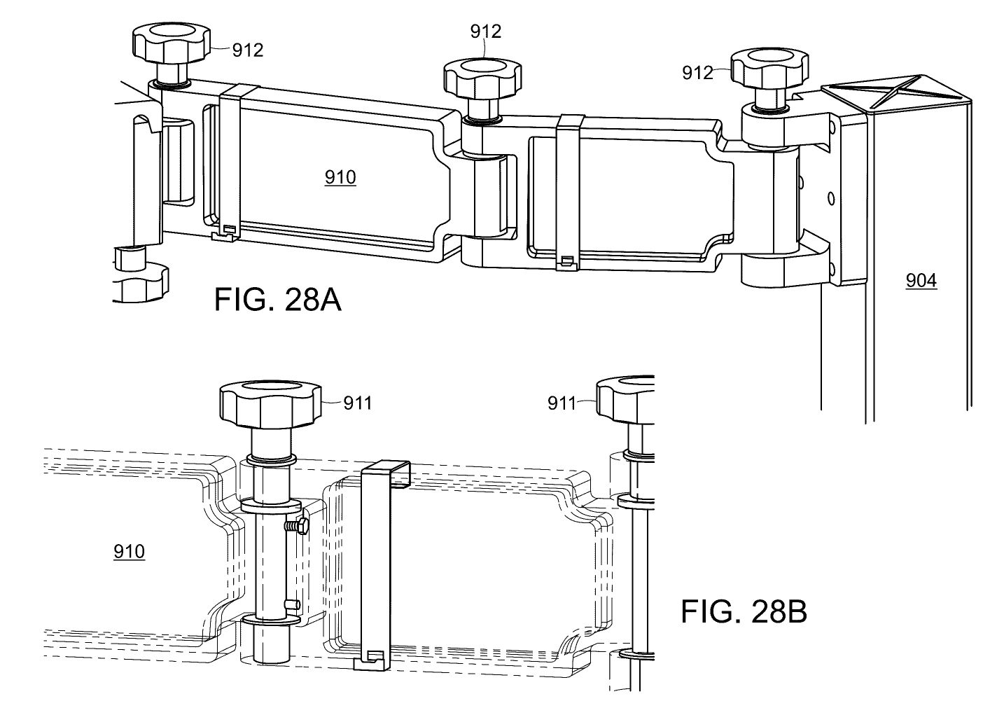

FIGS. 28A and 28B are enlarged graphical representations of SCARA arm adjustments 911 and SCARA arm 910 showing the adjustment of SCARA arm 910 in relation to macro height adjustment post 904 in FIG. 28A.

FIGS. 29A and 29B are enlarged front and back graphical representations of an embodiment of XYZ mounting system 915 for injection dispensing device 940 supported by horizontal support 920a and 920b and vertical support 926 comprising vertical adjustment rails 927 (FIG. 29B). Bracket 928 shown on FIG. 29A attaches to SCARA positioning arm 910 (not shown) and is locked in place by quick disconnect hand screw 929. Micro adjustment 931 provides for adjustment of the injection dispensing device 940 along the “z” axis by raising and lower support arm 920a and 920b along vertical rails 927 of the SCARA positioning arm 910 . Adjustment of microadjuster 931 and rotation of rotating platform 930 enable the proper orientation and positioning of injection dispensing device 940 with attached guide needle 942 for injection into the spinal cord of a subject. Adjustment of goniometer 950 by goniometer macroadjustment knobs 951 permits further adjustment of the angle of the guide needle 942 to allow the positioning of the beveled injection needle 943 (not shown) for entry into the spinal cord of a subject at the desired angle.

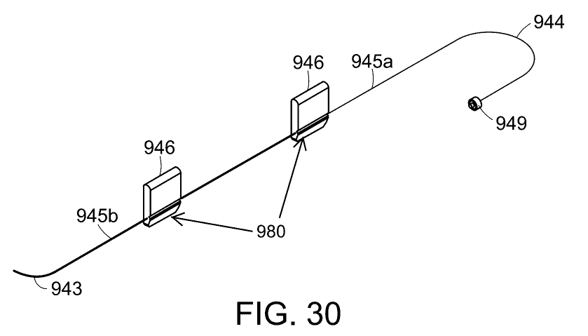

FIG. 30 is a graphical representation of a disposable trombone needle assembly comprising outer trombone sleeve 945a, inner trombone sleeve 945b, injection needle service loop 944, syringe connector 949 (for example a Hamilton RN or Luer connection), and a curved guide tube/needle 942. The trombone assembly 945 in combination with the delivery catheter/injection needle service loop 944 and curved guide tube/needle 942 provides a continuous conduit for delivery catheter/injection needle 943 (not shown) that is designed to prevent beveled injection needle 643 from kinking or jamming. In an embodiment the trombone assembly, injection needle and the curved guide needle are assembled and sterilized and manufactured as disposable assemblies. The entire needle guidance system comprising the curved guide tube/needle 942 and trombone assembly are internally sized to accommodate a 29 gauge Nitinol® (nickel-titanium alloy, oxide finish) cannula finished with a lancet point, or in some embodiments a blunt flexible catheter.

### Construction of the Trombone Assembly

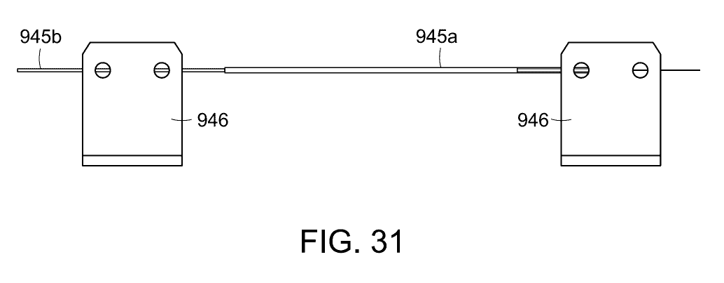

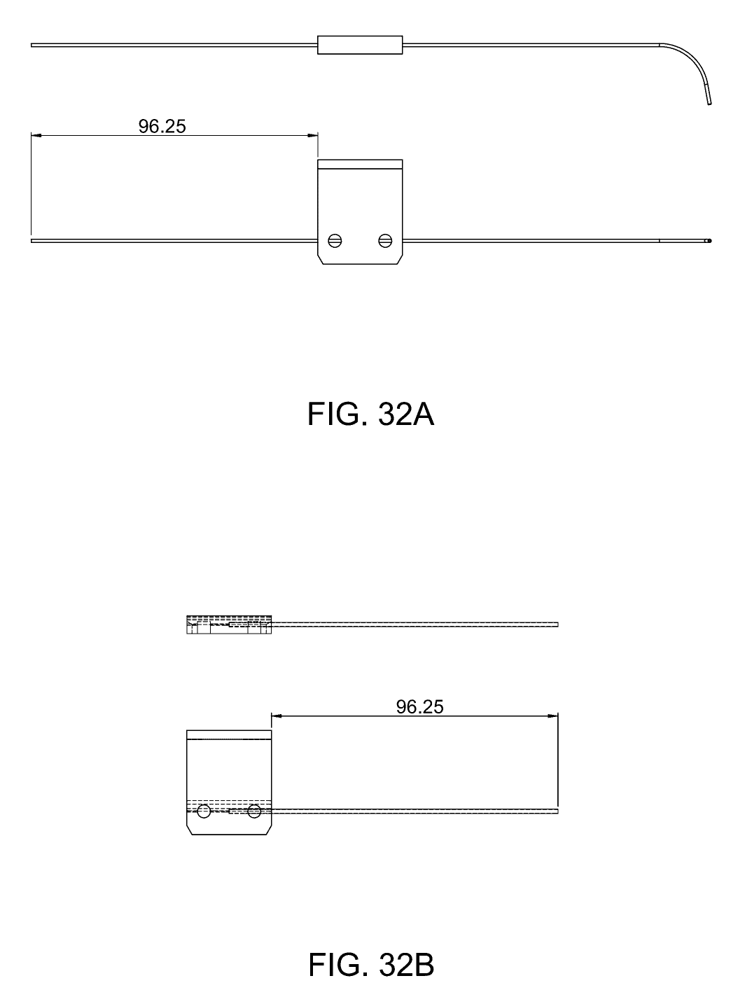

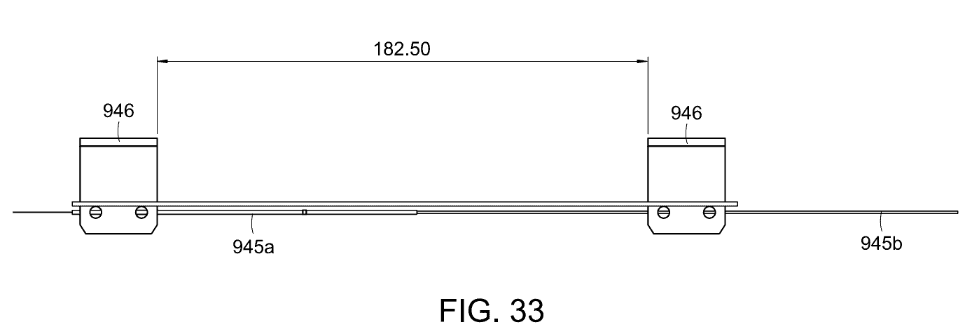

In a preferred embodiment, the construction of the trombone assembly requires assembly of two hypotubes made of 316 stainless steel such that the overlap of the outer trombone tube 945a and the inner trombone tube 945b are shown as in FIG. 31. The inner trombone tube 945b is fabricated from 316 stainless steel to have a blunt end (not shown) that fits snugly in the cannula of the outer trombone tube 945a. The opposite end of trombone tube 945b is curved and forms guide needle 943 (as shown in FIG. 30, i.e., 945b). Plastic snap-on tabs 946 with appropriately dimensioned slot 980 (as shown on FIG. 30) are appropriately sized to accommodate outer trombone tube 945a and inner trombone 945b. In an embodiment, the snap-on tabs are spaced 182.5 mm as shown on FIG. 33. The blunt end of the inner trombone tube is slid perpendicularly through the appropriately dimensioned slot 980 of plastic snap-on tab 946 to protrude from snap-on connector 946 for a distance of 96.25 mm (as shown in FIGS. 32 and 32B). The blunt end of the inner trombone tube 945b is secured to a plastic snap-on tab 946 and is secured with a suitable epoxy.

FIG. 36 is a graphical representation of programmable controller. Controller 990 has two basic functions: the controller 990 is used to control and display the position of the injection needle and to control and display (volume dispensed) the flow rate of composition of cells and/or therapeutic substance from the syringe, for example, a prefilled syringe in some embodiments. The skilled worker will understand how to program the programmable controller to perform the principal functions of advancing and retracting the needle and controlling the volume and flow rate of the contents of the injection syringe.

A typical algorithm would include powering on the controller 990 through pressing the power switch (not shown). The “LOAD” switch 991 is pressed position the injection needle and syringe plunger in the ‘home” position.

After attaching the injection needle assembly and syringe to the injection device, the operator inputs the needle speed (mm/s) and fluid rate (uL/mm) into the controller by pressing “SET NEEDLE SPEED” 992 and “SET FLUID RATE” 993. Flow rates may be calculated as uL of volume deposited per millimeter of injection needle travel. Therefore, in an embodiment, the fluid flow and injection needle advancement\retraction are coupled.

A typical value for needle speed is 0.5 mm/second. A typical value for fluid rate is calculated taking into consideration the following factors: a small amount of fluid is to be extruded while inserting the injection needle into the cord. This is denominated the “pre-flow” rate and it is typically set to 0.07 uL/mm. During retraction of the needle under actuation of the linear actuator by the controller, a rate of 0.34 uL/mm is typically used. Taking the foregoing into consideration, this would approximate 10 uL/min. when moving the injection needle at 0.5 mm/second.

An operator would next hold down the “FAST” button 994 and then press the advance “ADVANCE” button briefly until the tip of the injection needle is protruding from the guide needle (not shown). This allows the cell droplet to be visualized by the operator during priming of the syringe. Holding down the FAST 994 button accelerates the speed of the injection needle above the set needle speed. This would be done to perform quick movements of the injection needle.

The operator would next press the “PRIME” button to prime the syringe. This results in the syringe plunger moving at a rate of 20 uL/min, for example, a safe fluid flow rate for cells. The syringe is PRIMED until a drop of cells is visible.

The “FAST” button 994 is held and the “RETRACT” button 997 is pressed to retract the injection needle until it is just at the tip of the guide needle.

The “ZERO NEEDLE POSITION” button 998 is pressed to zero the needle position indicator. Next, the “ZERO FLUID VOLUME” button is pressed to zero the fluid volume delivered indicator.

By pressing “DISPENSING TOGGLE” 1100 flow of cells from the pre-filled syringe is commenced when the injection needle is in motion. There is an indicator light that turns on when the dispensing toggle is pressed. No cells are delivered if the dispensing toggle is not pressed.

When ready to perform injection: with dispensing toggle ON (if pre-flow of cells), the operator holds down the “ADVANCE” button 995 without holding the Fast button 994 to advance the injection needle into the cord at the set needle speed. The position of the needle is noted and the “ADVANCE” toggle 995 is released when at the desired needle position, typically about 20 mm.

The operator next presses the “SET FLUID RATE” button 993 and uses the keypad 1101 to change the fluid rate to the desired dispensing rate during needle retraction.

Next, the operator would hold down the “RETRACT” button 997 and retract the injection needle (a dispensing light is still on, so cells are being injected at the pre-set fluid rate). The needle position will return back to 0 mm when the needle is fully retracted.

Record the FLUID DELIVERED for documentation purposes. The fluid delivered increases whenever the system is injecting cells and/or a therapeutic substance, regardless of whether the needle is being advanced or retracted. Following administration of the cells and/or therapeutic substance from the pre-filled syringe, the “ SYRINGE RETRACT” button 1102 IS PRESSED to back up the syringe plunger and remove the syringe.

In case of emergency, pressing the “E-STOP” button 1103 stops the motors.

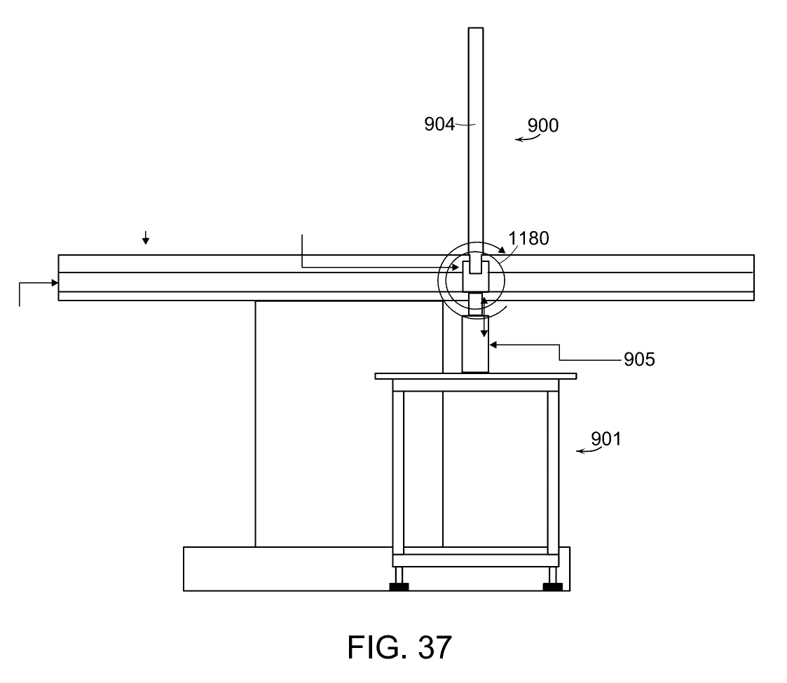

FIG. 37 is a graphical representation of an injection system positioned on a mobile cart and attached to an operating table or a surgical bed. The skilled person will recognize that various height adjustment mechanisms 905 may be utilized to raise the vertical or macro adjustment post 904, including cranks and gearing mechanisms. In some embodiments, such height adjustment mechanism may be motorized. Attachment 1150 may be a clamp, thumbscrew or vise-type mechanism, or equivalent. Cart 901 may be configured and sized to accommodate various surgical beds and operating tables and may have wheels and locking mechanisms (not shown).

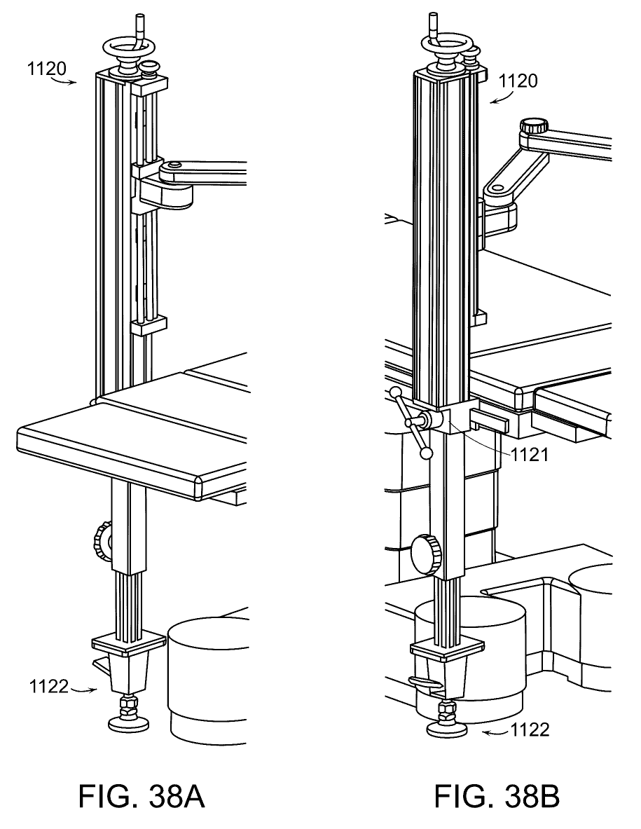

FIG. 38A and FIG. 38B are graphical representations of a monopod support for an embodiment of an injection device attached to an operating table or a surgical bed. In some embodiments the bottom of the monopod may have height adjustment mechanisms 1122 with a pedal to provide additional stability to the monopod vertical adjustment post 1120. The monopod may be attached to a bed rail through vice-like connection 1121.

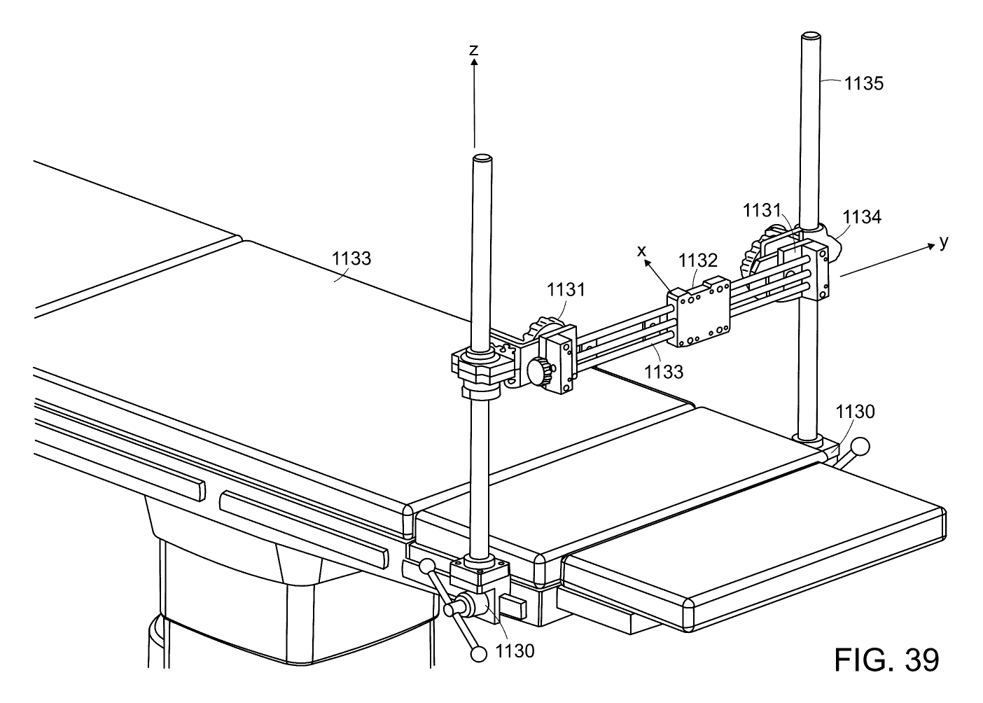

FIG. 39 is a graphical representation of a bridge bed rail for support of an injection device. This embodiment employs horizontal adjustable support 1131 comprising one or more rails 1133. Sliding mounting platform 1132 is capable of moving laterally along rail(s) 1133 to provide adjustment along the y axes. This embodiment shows two vertical height adjustment posts 1135 having sliding and lockable mountings 1134. In this manner, adjustments may be made in along the z axis. Mounted to sliding bracket 1132 is a corresponding mounting affixed to trail injection dispensing device (not shown), which is adapted to comprise an adjustable arm to enable adjustment of the injector along the x axis. The height adjustment posts 1135 are locked to opposite rails of the surgical bed or operating table 1133 with vice-like clamps 1130, or an equivalent attachment.

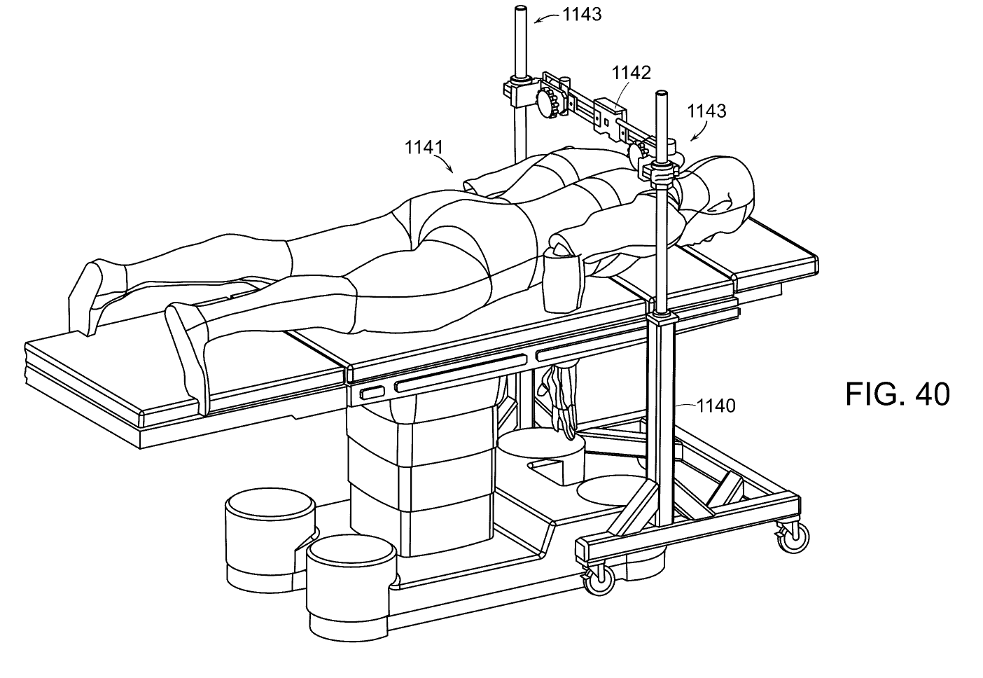

FIG. 40 is a graphical representation of a cart bridge support 1140 for an injection device positioned over a human subject 1141 positioned prone on an operating table or surgical bed. In this embodiment vertical adjustment posts 1143 support a horizontal positioning system as described in FIG. 39.

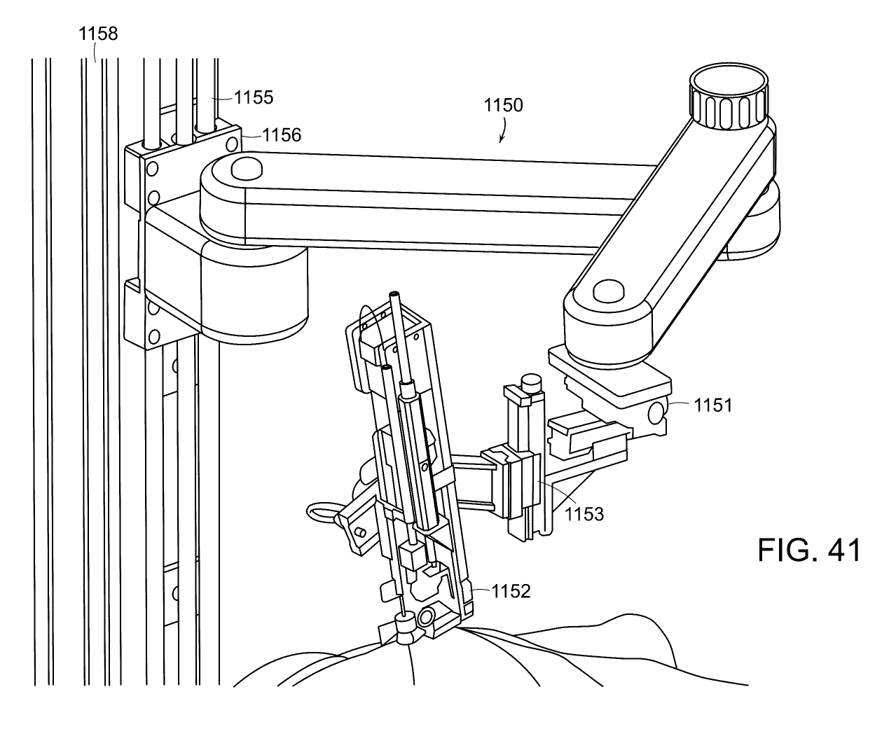

FIG. 41 is a graphical representation of a SCARA positioning arm 1150 of an embodiment. In this embodiment vertical height adjustment post 1158 may contain one or more vertical rails 1155 that slide along rails 1158 and are attached to SCARA positioning arm 1150. This configuration allows for adjustment along the x axis. SCARA arm may be adjusted in use to provide adjustments along the x and y axes in use over a prone human subject. Micro-adjustment 1153 provide for precise micro-adjustments along the z axis in use.

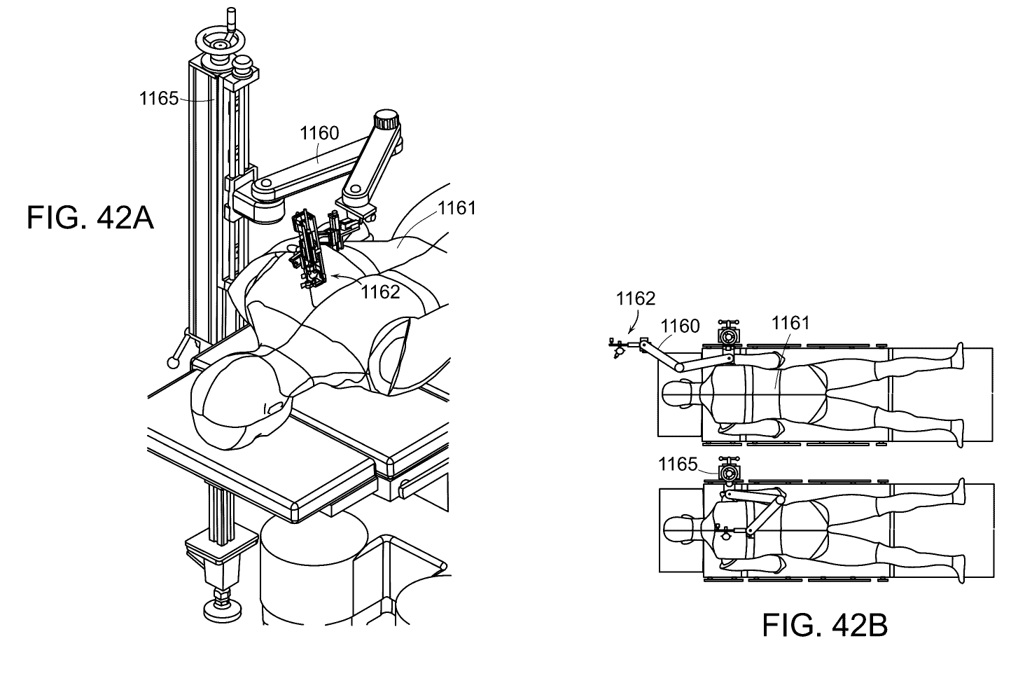

FIG. 42 is a graphical representation of the positioning of the SCARA positioning arm 1160 and injection system 1162 in use positioned over a human subject 1161 in the prone position on an operating table or surgical bed.

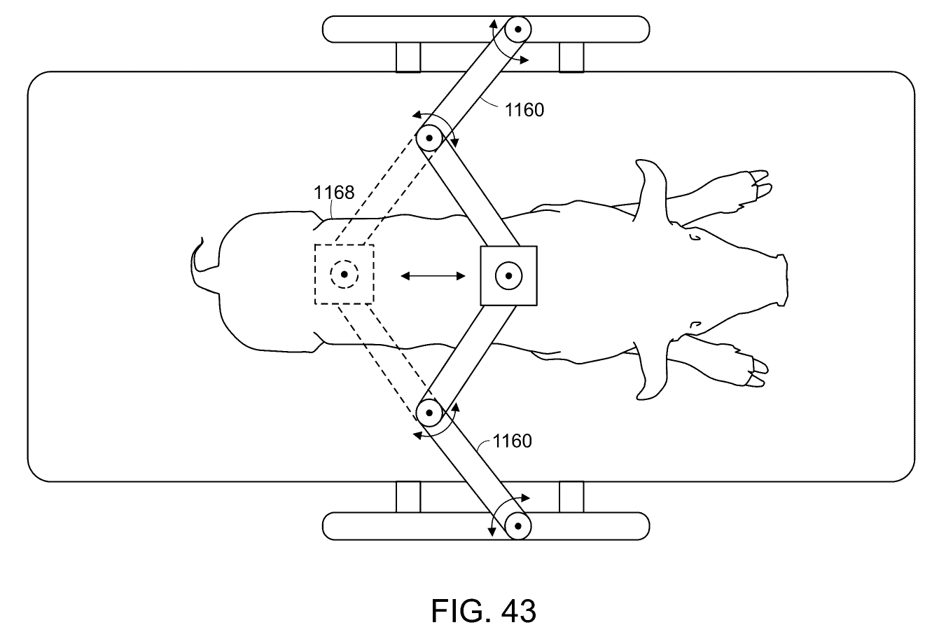

FIG. 43 is an illustration of a dual SCARA positioning arm support 1160 for an injection device positioned over an animal subject 1168.

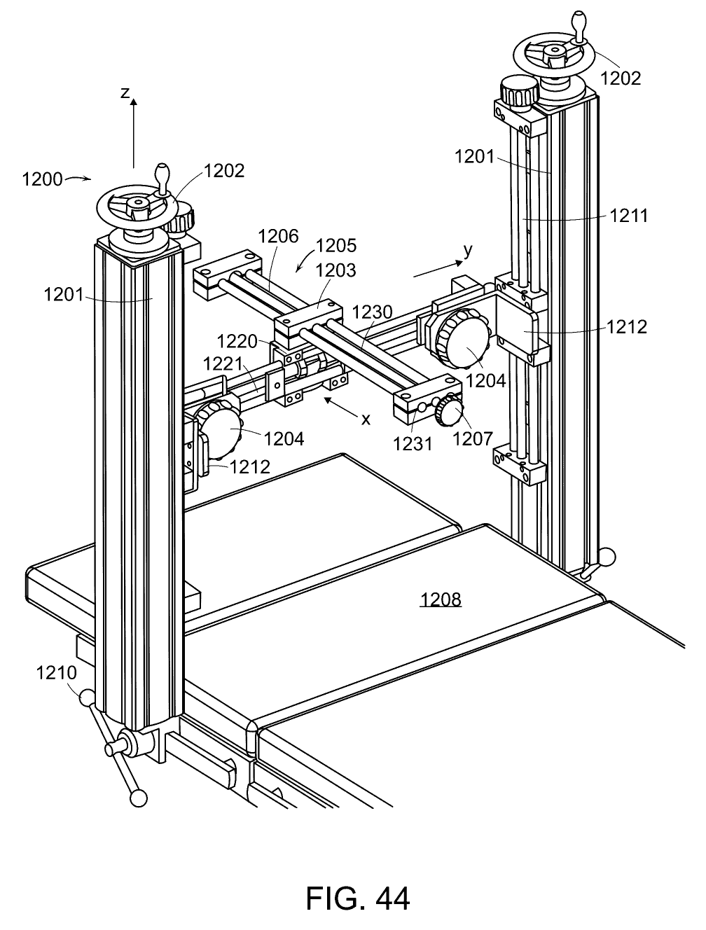

FIG. 44 is a graphical representation of an injection device 1201 mounted along the rails of a surgical bed 1208 with clamps 1210 for positioning the injection device above the surgical bed or operating table. The illustration depicts an embodiment of an XYZ mounting system 1205. Height adjustment is achieved in the vertical direction by slidable brackets 1212 sliding vertically along rails 1211 controlled by vertical height adjustment knobs 1202. This enables adjustment of the injection system along the z axes. Adjustment along the x and y axes is accomplished through sliding platform 1220 laterally along rails 1221 thereby controlling position along the y axis. Adjustment 1207 allows for movement of bracket 1231 along rails 1230 in the x axis which are secured by block 1203. Bracket 1231 may then support an injection system (not shown).

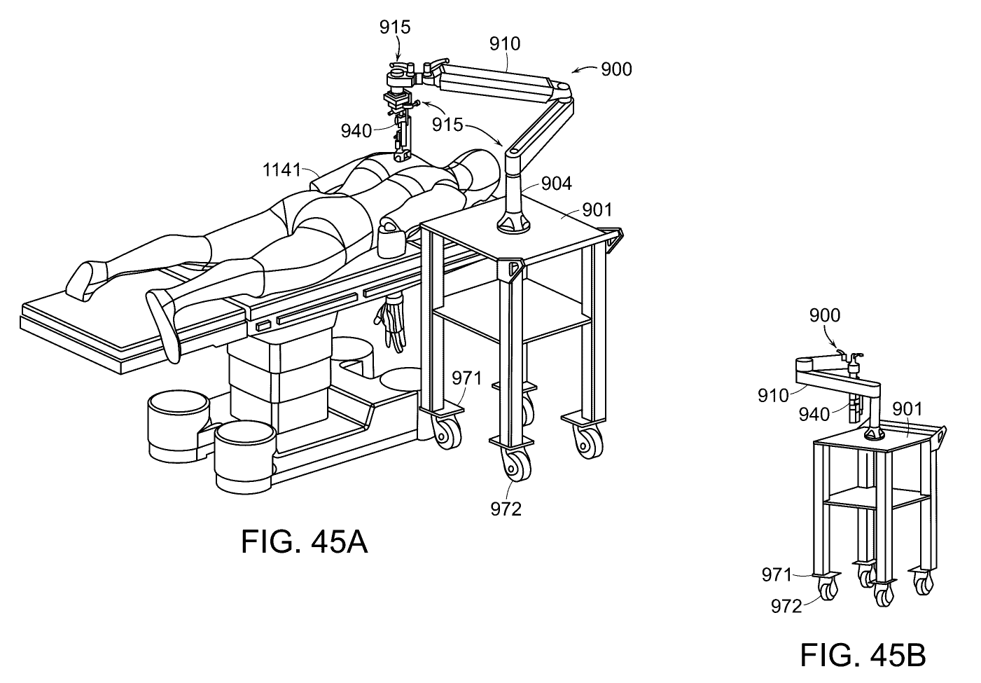

FIG. 45A and 45B are graphic representations showing an embodiment of an XYZ positioning system and a mobile cart, respectively. XYZ positioning system 915 utilizes a vertical height adjustment post 904 and a SCARA positioning arm 910 to support injector dispensing device 940 positioned over a human subject in the prone position 1141. Mobile cart 901, as shown, may have wheels 972 and locking mechanism 971 to position the mobile cart supporting injection device 900 firmly alongside an operating table or surgical bed.

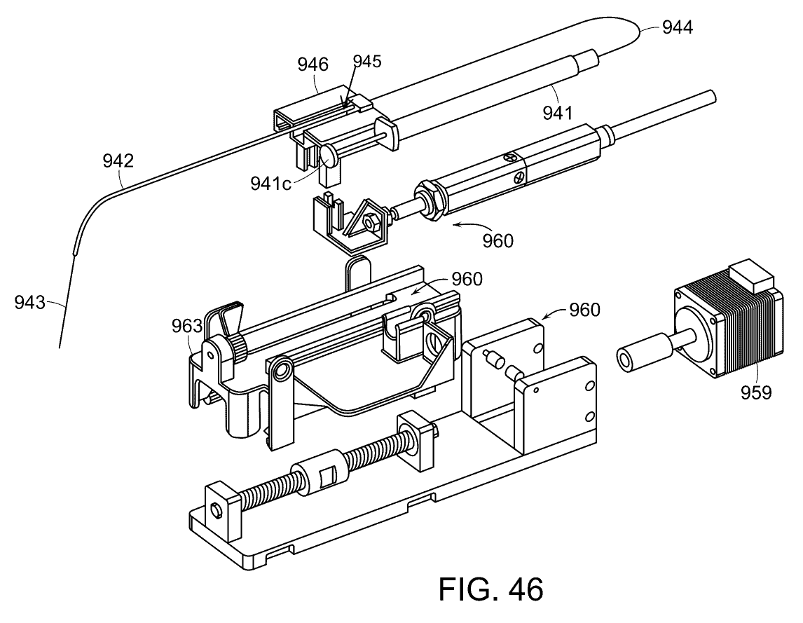

FIG. 46 is a graphical representation of an embodiment of the telescoping cannula/trombone mechanism 942 and the motorized linear actuator syringe mechanism 960 for actuating the plunger rod 941c and delivery catheter/injection needle 943 . An exploded view of a motorized syringe mechanism 960 in communication with linear actuator 959. Plunger driver 963 controls movement of plunger rod 941c of the syringe 941. Also shown is an embodiment of mounting support block 946 firmly attached to telescoping cannulas (trombone mechanism) 945 terminating in guide needle 942. Injection needle 943 is joined at one end to syringe 941 and terminates in a bevel (lancet shape or otherwise) at the end emerging from guide needle 943.

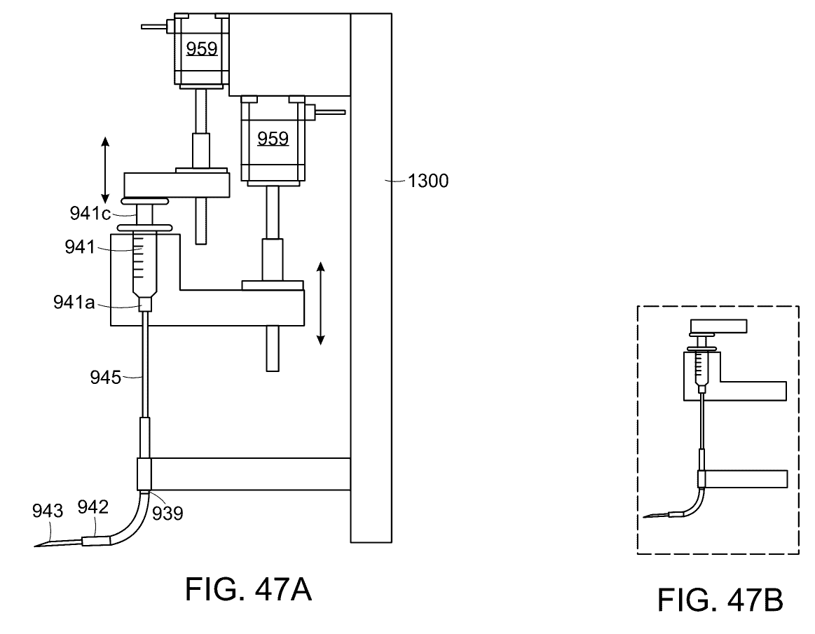

FIGS. 47A and 47B are graphical representations of an embodiment of the delivery catheter/injection needle 943 illustrating the mechanized actuation of the syringe plunger rod 941c and delivery catheter/injection needle 943 through the guide tube or introducer needle 942. Linear actuators 959 control movements of the syringe 941 and plunger rod 941c and delivery catheter/injection needle (not shown).

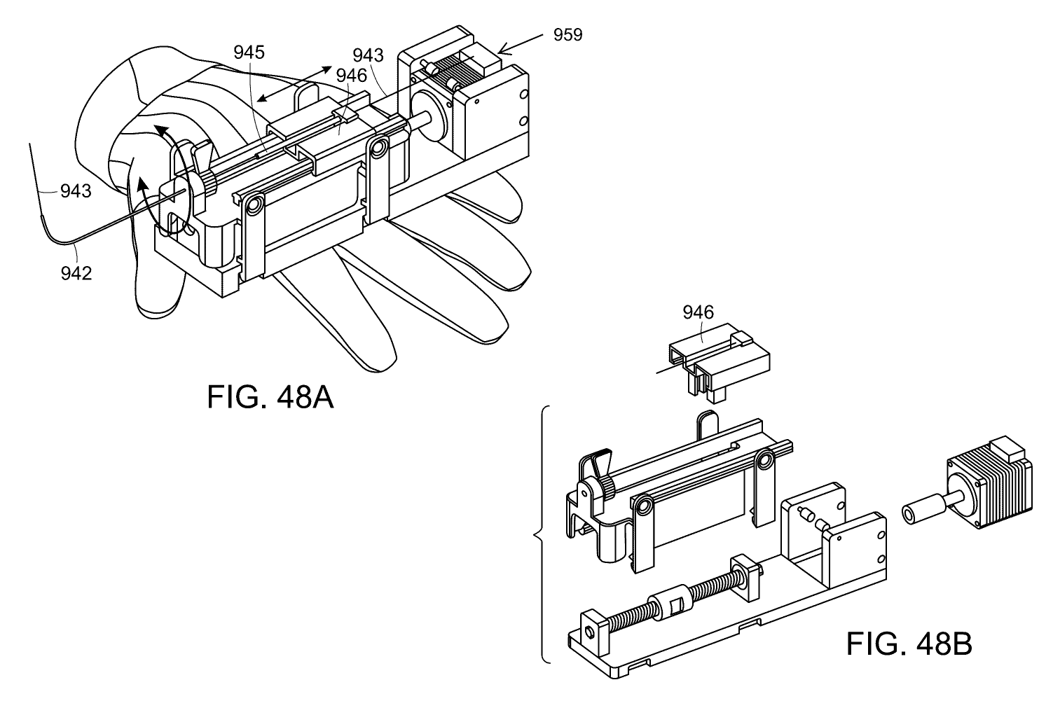

FIG. 48 is a graphical representation of a disposable telescoping guide tube/trombone assembly 945 for attachment to a prefilled syringe (not shown) . The trombone assembly includes two-part telescoping cannulas (trombone mechanism) 945 firmly supported to mounting block 946. Shown also guide tube/needle 942 and delivery catheter/injection needle 943. The assembly is attached to linear actuator 959 (in this embodiment a stepper motor).

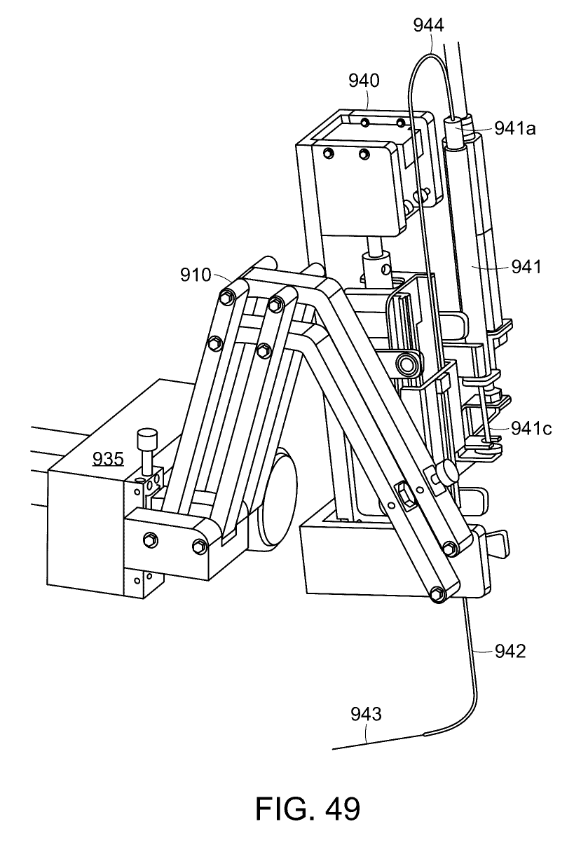

FIG. 49 is a graphical representation of an embodiment of a goniometer-like angle control mechanism. This allows for manipulation of angle around the tip of the guide tube/needle 942 housing delivery catheter/injection needle 943. The angle positioning mechanism 910 supports injector dispensing device 940 bearing syringe 941 and showing syringe connector 941a and plunger rod 941c. Delivery catheter/injection needle service loop is shown as 944. The SCARA positioning arm is attached to adjustable bracket 935.

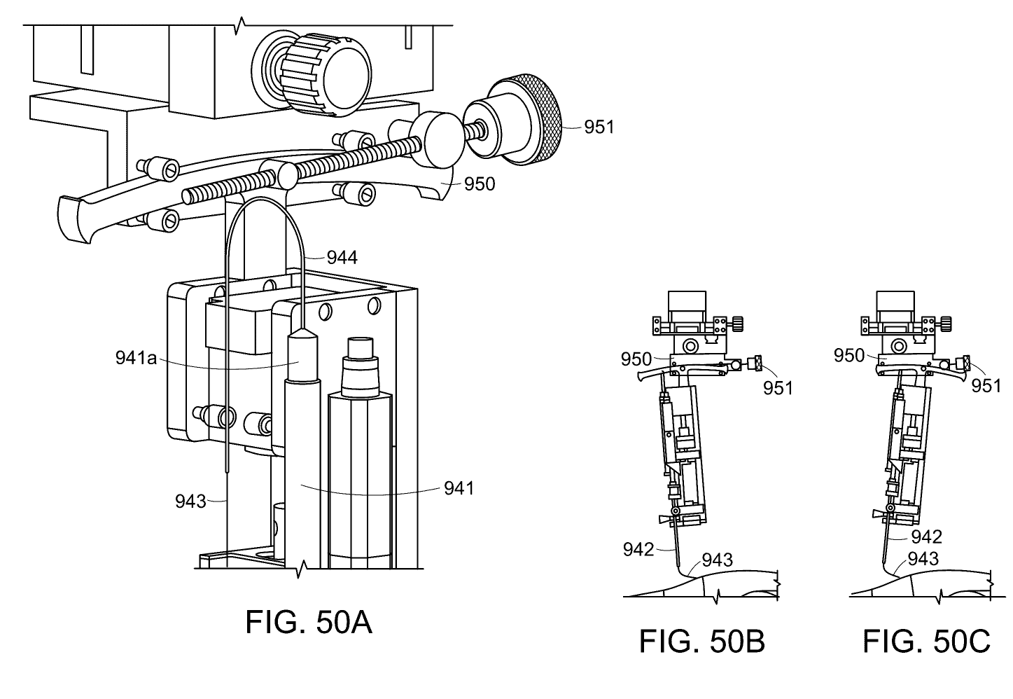

FIGS. 50A, 50B, and 50C are graphical representations of an embodiment of an adjustable goniometer 950 for controlling pitch of the guide tube/needle and delivery catheter/injection needle. Goniometer 950 is adjusted through screw adjustment 951.

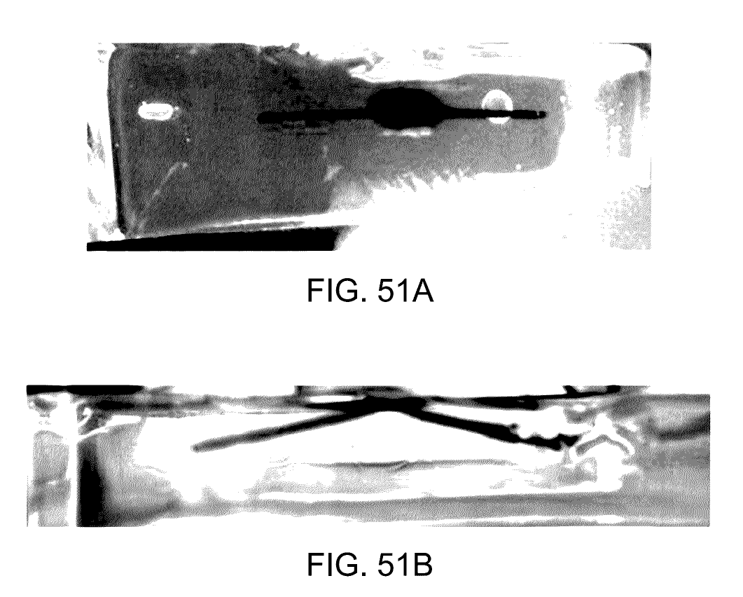

FIG. 51A and 51B are photographs of methylene blue stained hyaluronic acid trails injected at an angle in a “tent” formation (from above in FIG. 51A) and from the side (FIG. 51B) around a prophetic anatomical space injection site.

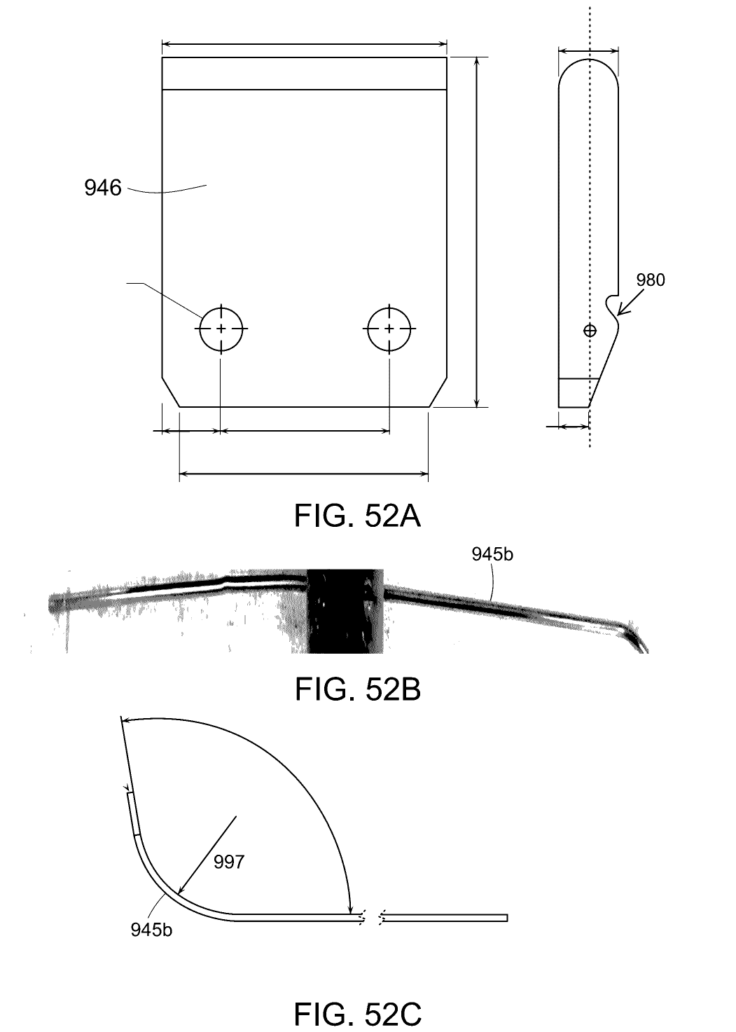

FIG. 52A provides a graphic representation of and attachment block 946 to which a telescoping cannula assembly (trombone assembly) 945a may be attached, in one embodiment, by an epoxy adhesive in slot 980.

FIG. 52B is a photographic showing lower trombone cannula 945b attached by an epoxy adhesive to attachment block 946.

FIG. 52C is a graphic representation of lower trombone cannula 945b showing a 100° bend angle.

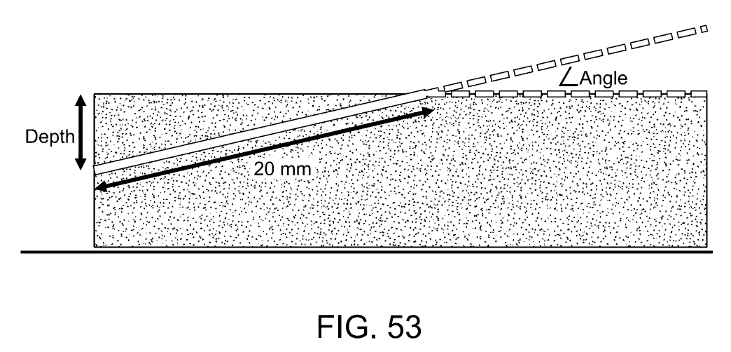

FIG. 53 depicts the angle measurements in accordance with the injection of a 20 mm trail of the liquid composition of HA and methylene blue in accordance with this Example 3.

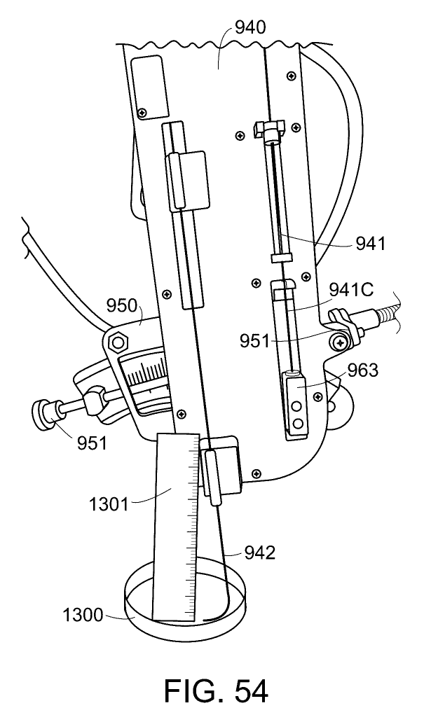

FIG. 54 depicts the testing setup for injection device 900 used in this Example 3. FIG. 54 is an image of injector dispensing device 940 with guide needle 942 positioned over agarose gel slap 1300. As part of injector dispensing device 940, syringe 941 with plunger rod 941c positioned within motorized plunger drive 963 is depicted. Also, depicted is goniometer 950 and microadjustment knobs 951. Ruler 1301 is used to measure the trails of HA and methylene blue (not shown) in agarose gel slab 1300.

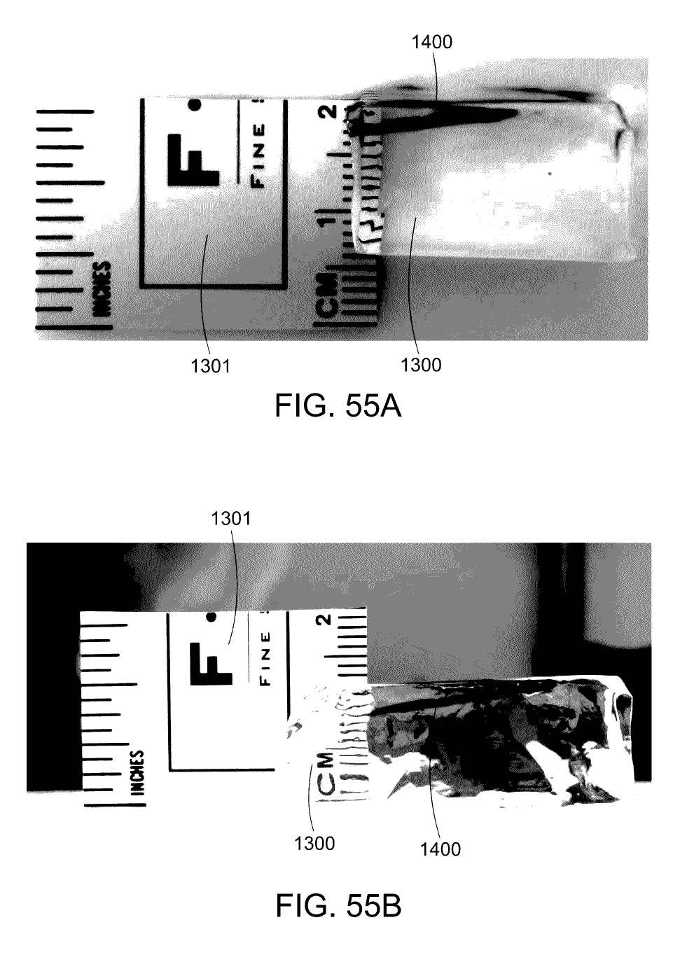

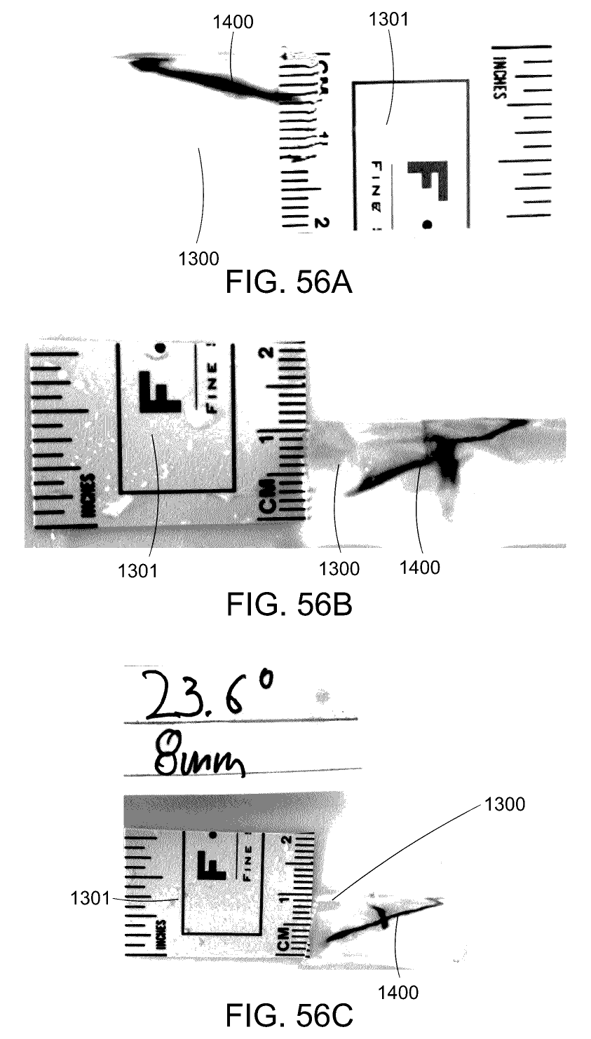

FIGS. 55A and 55B are images of methylene blue trails from a liquid composition comprising HA and methylene blue injected into an agarose slab at a setting of 2 mm and an injection angle of 5.7° in accordance with Example 3. FIGS. 56A, 56B and 55C are images of methylene blue trails from a liquid composition comprising HA and methylene blue injected into an agarose slab at a setting of 4 mm at an injection angle of 11.5°, 6 mm at an injection angle of 17.5° and at 8 mm at an injection angle of 23.6° in accordance with Example 3.

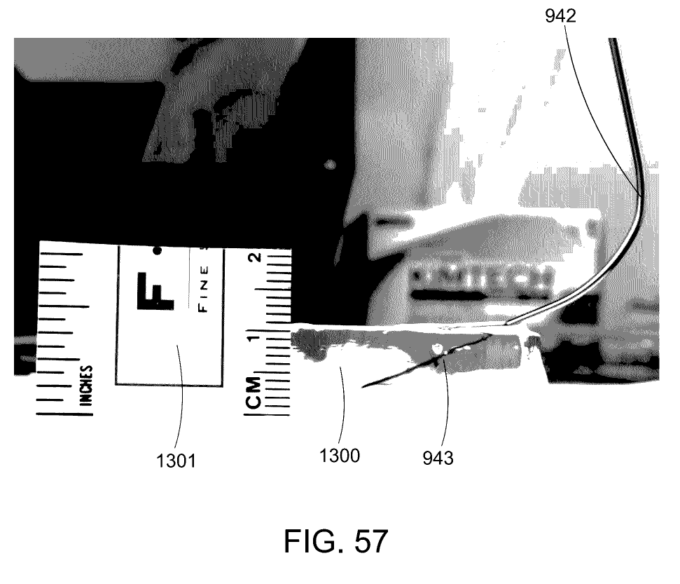

FIG. 57 is an image of a guide tube/needle positioned at the surface of an agarose gel slab and an injection needle 943 penetrating the agarose gel slab 1300 at a setting of 8 mm and an injection angle of 23.6° yielding a trail of 8 mm in accordance with Example 3.

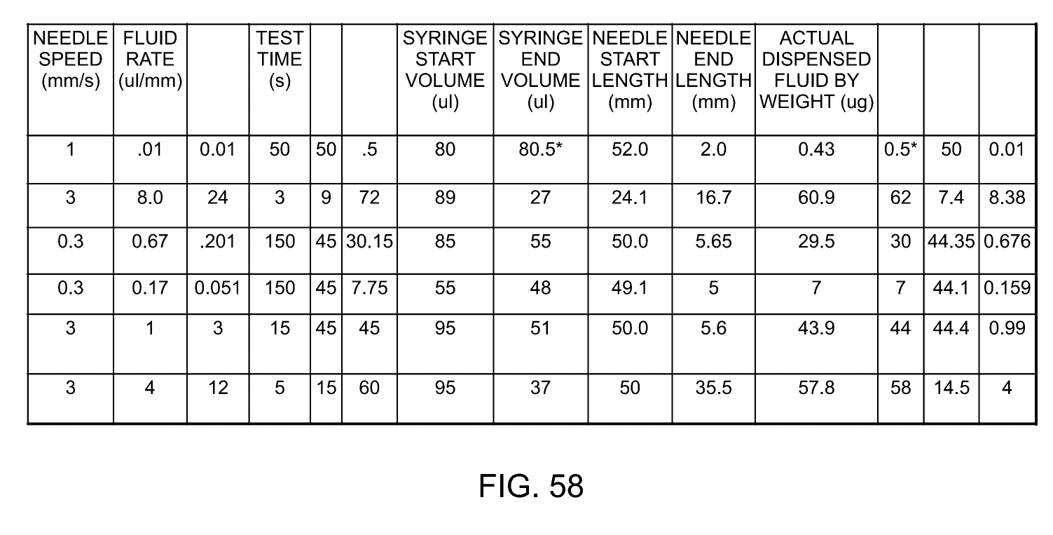

FIG. 58 contains data from Example 5 including actual and expected fluid rates, needle travel and total dispensed volume.

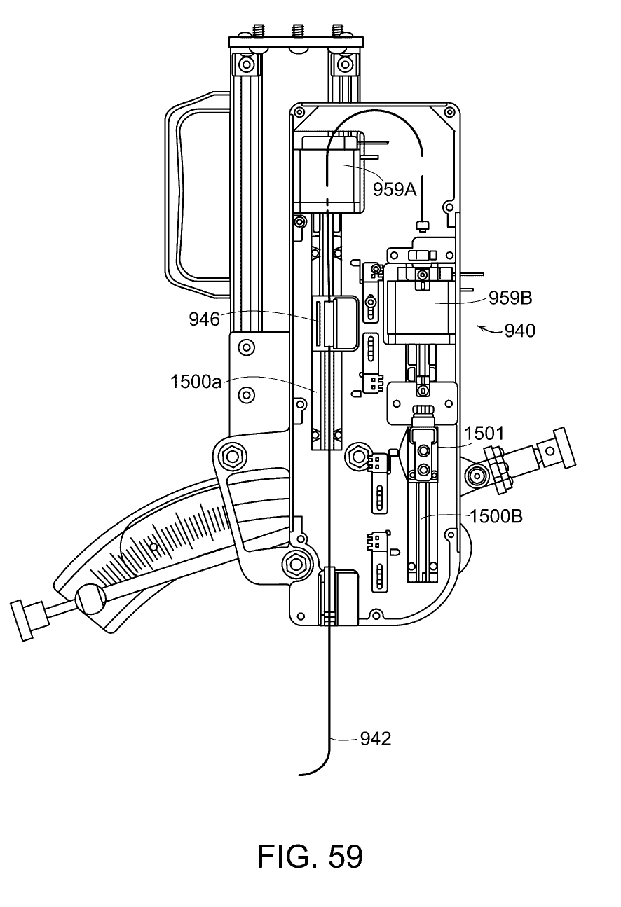

FIG. 59 is a graphic representation of an injector dispensing device 940 and in a preferred embodiment two linear actuators 959a and 959b. Linear actuator 959a (in this embodiment a stepper motor) actuates mounting block 946 secured to upper trombone cannula 945a which is secured to a proximal surface of delivery catheter 942. Actuation of mounting block 946 and secured trombone tube 945a thereby advances and retracts delivery catheter/injection needle 943 through lower trombone tube 945b and the distal end of guide tube 942 depicted with a bend at the distal end.

Linear actuator 959b drives carriage 1501 along one or more rails 1500b to control the movement of the plunger rod of the syringe. Linear actuator 959b may be configured with moveable stage 1501b driven by linear actuator 959b along one or more rails 1500b to actuate the plunger rod to deliver a liquid composition from a prefilled syringe 941 (not shown). A stop switch or equivalent is incorporated (not shown) to prevent over travel of stage 1501 along rails 1500b. It will be appreciated by the skilled person that while two linear actuators are shown in a preferred embodiment of the present invention, a single linear actuator could be configured to perform each function independently under the control of programmable controller 990.

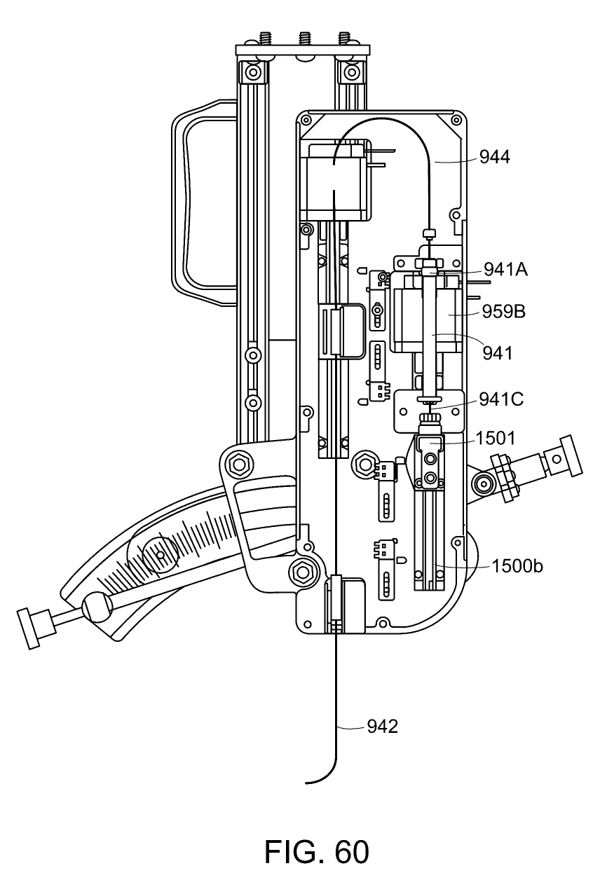

FIG. 60 is a graphic representation of preferred embodiment of an injector dispensing device, as discussed in connection with FIG. 59 above, with a syringe 941 attached. Syringe connector 941a connects to delivery catheter/injection needle configured to provide service loop 944. The plunger rod 941c of syringe 941 is actuated by the moveable stage 1501 by linear actuator 959b by moving stage 1501 along rails 1500b.

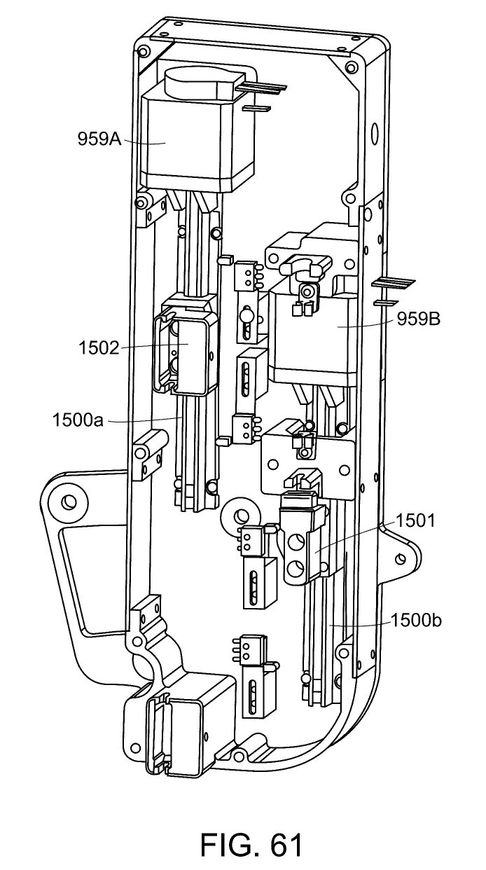

FIG. 61 is a graphic representation of an exploded view of injector dispensing device 940 showing in more detail the positing of linear actuators 959a and 959b in a preferred embodiment

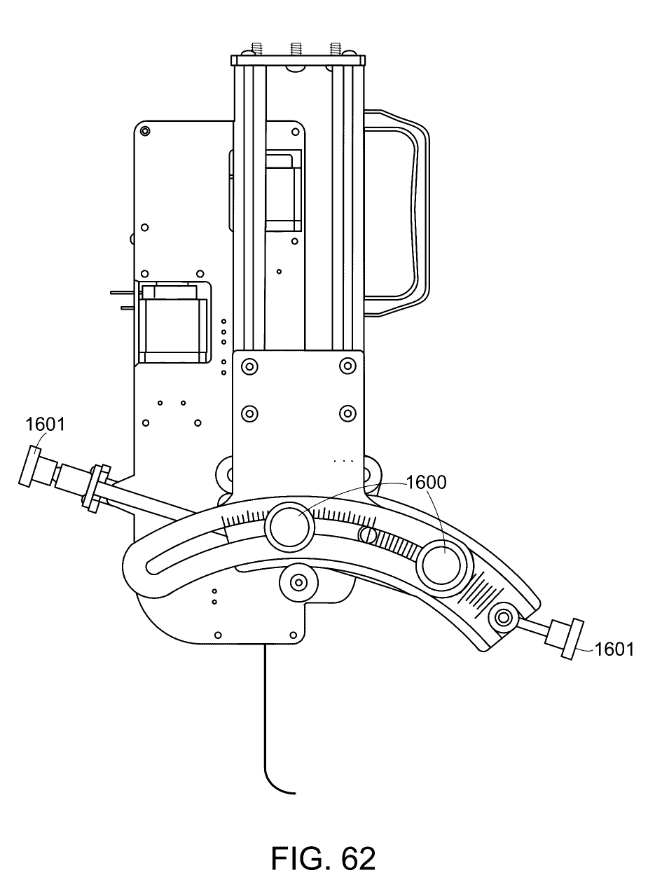

FIG. 62 is a graphic representation of a goniometer 950 used in a preferred embodiment of the injector dispensing device 940. A preferred embodiment of goniometer is shown depicting macro-angle adjustments 1600 and micro-angle adjustments 1601. It will be appreciated by the skilled worker that the macro-angle adjusters 1600 and micro-angle adjusters 1601 may be configured in a number of ways to provide the same function as those depicted in FIG. 62.

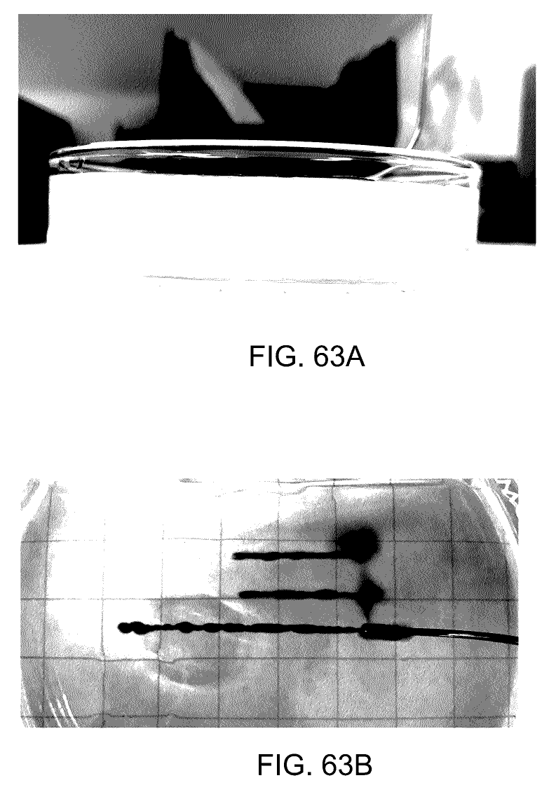

FIGS. 63A and B are photographs depicting three trails of hyaluronic acid-methylene blue in agarose demonstrating consistent trail angles in FIG. 63B.

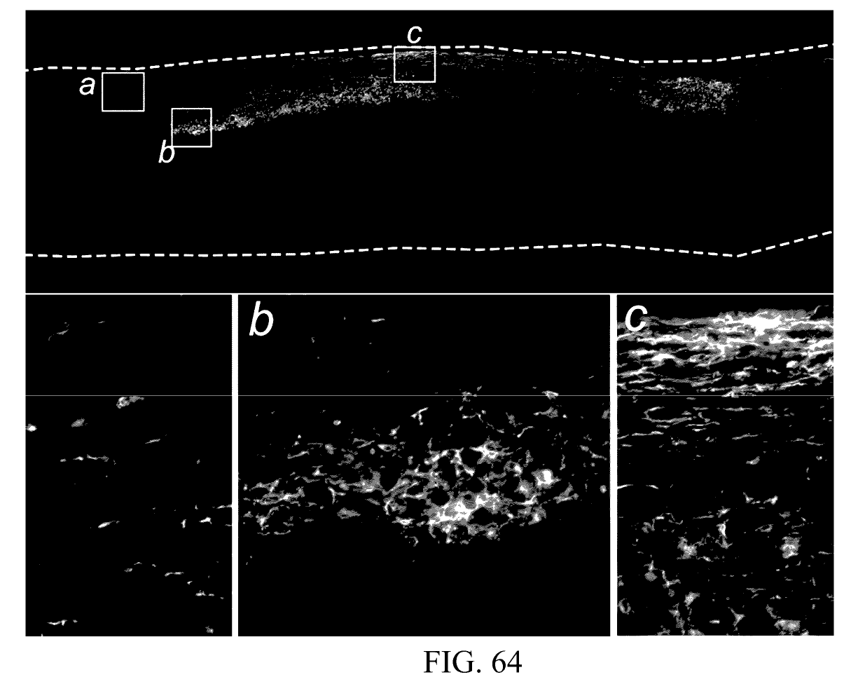

FIG. 64 shows human neural stem cells delivered in a trail into a nude rat spinal cord after one month. The cells were labeled for STEM121 and doublecortin (DCX) markers, showing cell survival and neuronal precursor differentiation.

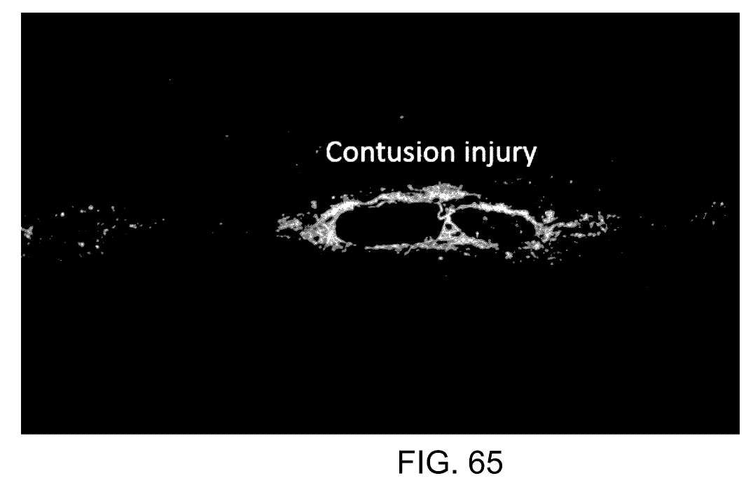

FIG. 65 shows survival of STEM121 labeled human neural stem cells delivered in a trail through a contusion injury in a nude rat. This demonstrates that cell trails can bridge injuries in the spinal cord and survive.

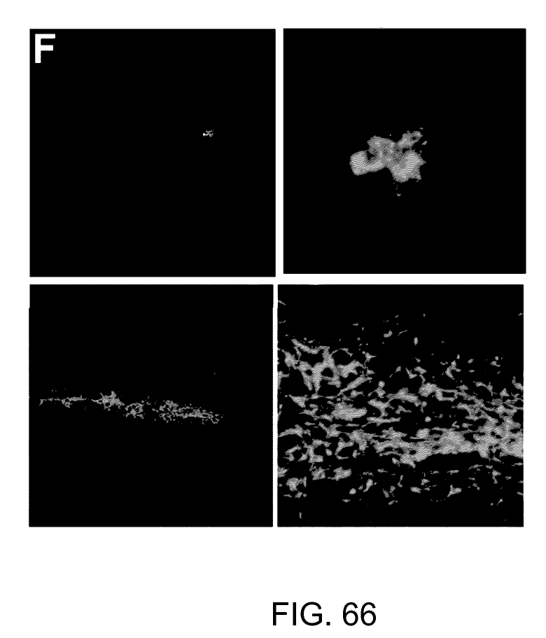

FIG. 66 shows cross-sections and longitudinal sections of STEM121 labeled human neural stem cells after 1 week delivered in a trial in a porcine spinal cord.

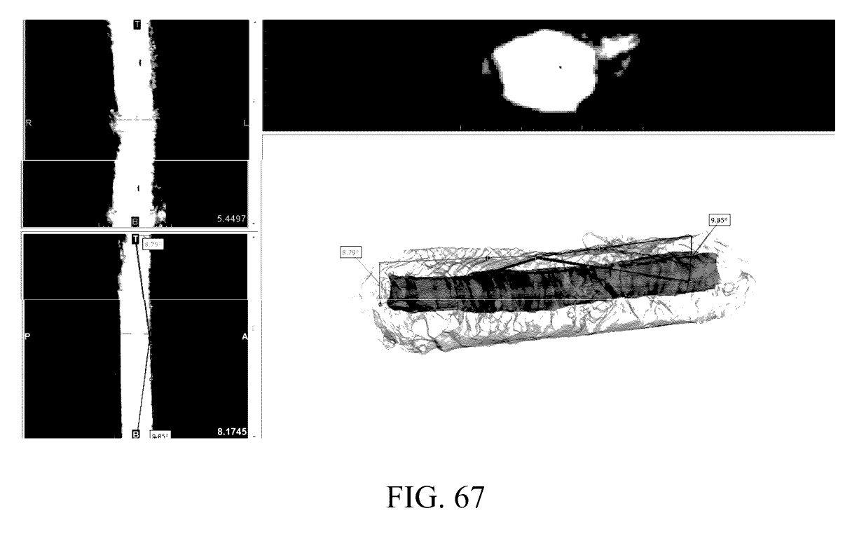

FIG. 67 shows a photograph of a disposable trombone assembly with polymeric polyethylene tubing (PE-5 catheter) extruding from the curved guide needle.

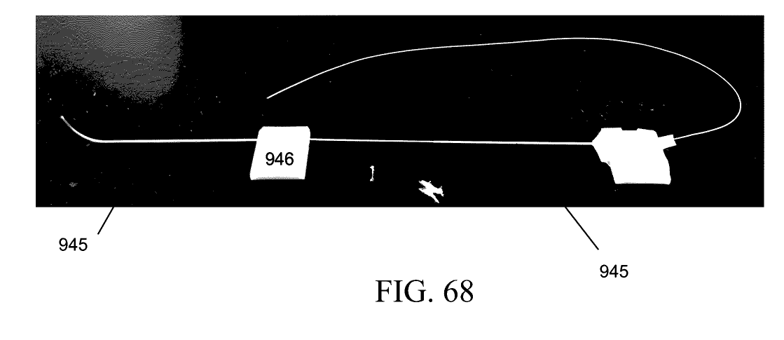

FIG. 68 shows a photograph of a trombone assembled fitted with polyethylene tubing (PE-8 catheter) secured with snap-on connectors to the linear actuator and fixed connector portion of the injection system. Methylene blue solution was loaded into the attached syringe and flowed through the PE-8 tubing.

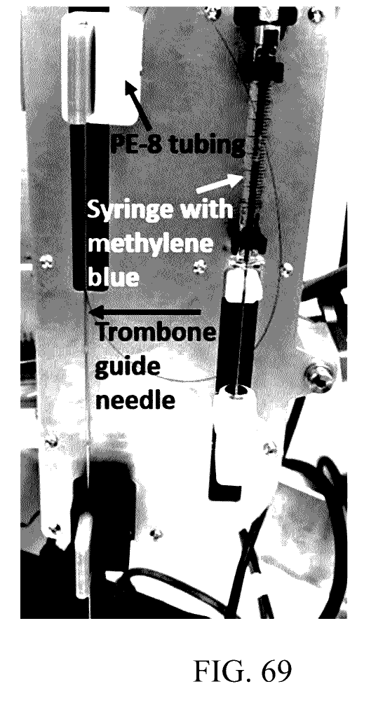

FIG. 69 shows photographs of a polyethylene catheter (PE-8) extruded through the guide tubing and into the subpial space of a rat (not shown) for injection of therapeutics.

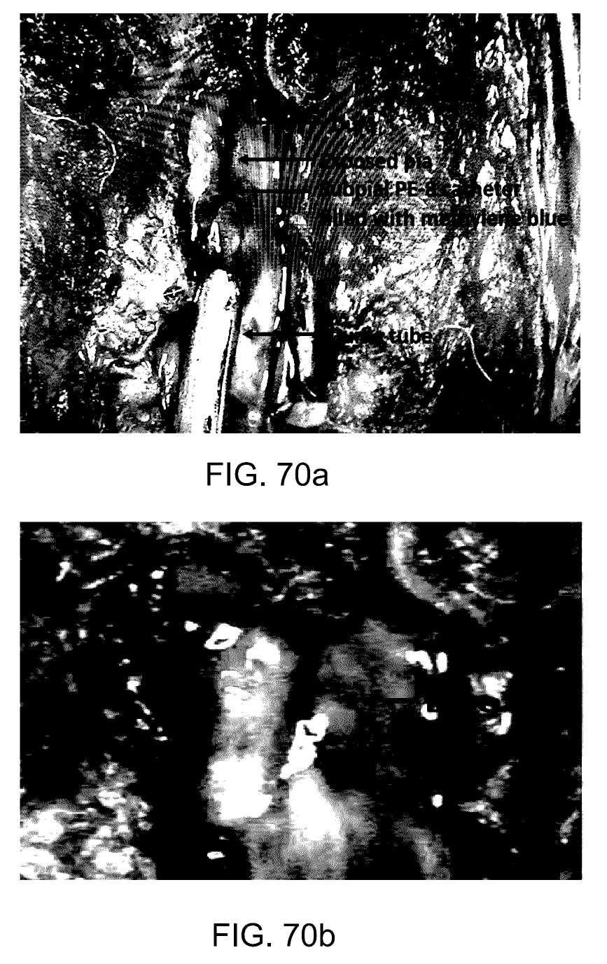

FIG. 70A shows a photograph of the PE-8 catheter in the subpial space and FIG. 70B shows a photograph of methylene blue injected through the catheter into the subpial space.

### EXAMPLES

### Example 1

### Operation of Experimental Injection Device in Surgical Setting

The present invention may is used to perform an experimental injection of neural stem cells into the spinal cord of pigs according to the following protocol. A portable, experimental injection device is constructed in accordance with the specification and figures, set forth herein. Three Yucatan mini-pigs of 20-25 kg are injected using a preferred embodiment of the present invention. Each pig receives a thoracic T10 laminectomy according to procedures well known in the art. No myelotomy is performed. The pia is nicked with a needle at the site of entry of the injection needle of the experimental injection device. The injection needle utilized in the trial is composed of Nitinol® (nickel-titanium alloy), hereinafter referred to as “Nitinol needle.”

The injection utilizes an aqueous composition of hyaluronic acid [0.75% w/v in divalent ion-free phosphate buffered saline] and human neural stem cells [StemPro, ThermoFisher Scientific] at a concentration of 100,000 cells/μL.

A 2 cm trail of cells in the spinal cord of each experimental mini-pig at a concentration of 100,000 cell/μL will be deposited, using the following administration parameters.

TABLE 1
Injection Administration Parameters

| | 2 cm Trail |
|---|---|
| Nitinol insertion & retraction rate | 0.5 |
| (mm/sec) | |
| Insertion - fluid delivery volume (μL/ | 0.07 |
| mm) | |
| Retraction - fluid delivery volume (μL/ | 0.34 |
| mm) | |
| Total injection volume (μL) | 8.2 |
| Total trail length (mm) | 20 |
| Total injection time (seconds) | 40 |

In the first experimental pig, a T10 laminectomy is performed according to conventional surgical procedures known in the art. A cell trail will be administered in the manner outlined in FIG. 34A. Trail 1: Stereotactic placement of a trail 1-2 mm right or left of midline and extending at ˜99 degrees for 2 cm (hypotenuse) in a caudal to rostral direction, ideally beginning (at maximal Nitinol extension) 2 mm above the most ventral aspect of the cord. Trail will begin in dorsal caudal white matter, travel rostrally and end in grey matter.

In the second experimental pig, a T10 laminectomy is performed according to conventional surgical procedures known in the art. A cell trail will be administered in the manner outlined in FIG. 34B. Trail #1 and 2: Stereotactic placement of a trail 1-2 mm right or left of midline and extending at ˜99 degrees for 2 cm (hypotenuse) in a rostral to caudal direction, ideally beginning (at maximal Nitinol extension) 2 mm above the most ventral aspect of the cord. Trail will begin in dorsal caudal white matter, travel caudally and end in grey matter.

In the third experimental pig, a T10 laminectomy is performed according to conventional surgical procedures known in the art. A cell trail will be administered in the manner outlined in FIG. 34C. Trail #1 and 3: Stereotactic placement of a trail 1-2 mm right or left of midline and extending at ˜99 degrees for 2 cm (hypotenuse) in a caudal to rostral direction. Trail will begin in dorsal caudal white matter, travel rostrally and end in grey matter. Trail #2 and 4: Stereotactic placement of a trail ending 2 mm (along the hypotenuse) beneath the dorsal surface of the cord 1-2 mm right of midline and extending at ˜99 degrees for 2 cm (hypotenuse) in a rostral to caudal direction. Trail will begin in dorsal caudal white matter, travel caudally and end in grey matter.

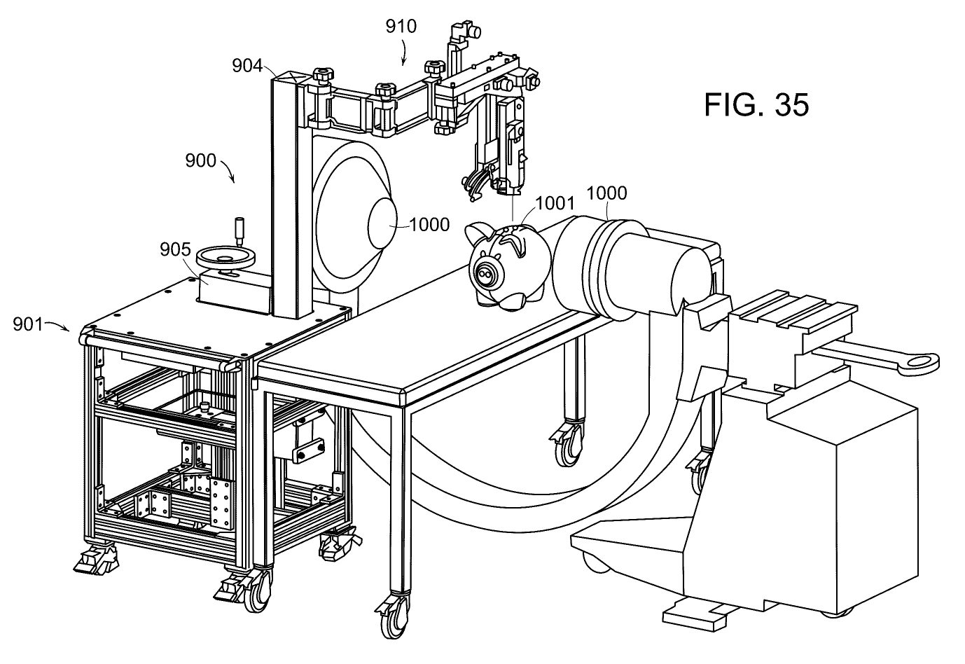

The administration of the human neural stem cells to the three experimental pigs will follow the following general procedure. A C-fluoroscope 1000 is positioned so as to allow lateral imaging of the cord by a radiologist. The experimental trail injection device 900 of the type depicted in FIG. 18 mounted on portable cart 901 is positioned next to operating table and checked for clearance by raising vertical macro height post 904 by manipulating macro height adjustment 905), and to determine the ability of SCARA positioning arm 910 to reach into the surgical\fluoroscope field, as graphically depicted in FIG. 35. Connection of the power source (not shown) and cable connection (not shown) of the motorized injection needle 960 to the motor box (not shown) is confirmed. The previously sterilized nitinol\guide needle assembly is flushed with sterile saline and checked for leaks. The experimental trail injection device 900 is powered-up by running the start-up procedure.

Next, the Nitinol injection needle 943 (not shown)\guide needle 942 assembly is secured to the motor assembly, as generally set forth in FIG. 19. A Hamilton syringe 941 is placed into the pump portion of motor syringe mechanism 960 and connected to the nitinol delivery catheter/injection needle. Infusion parameters are then programmed into control box 990 (See. FIG. 36). The dura of each experimental pig is tacked back by the surgeon according to conventional surgical procedures.

Thereafter, the Nitinol needle 943 is primed with the aqueous composition comprising hyaluronic acid and human neural stem cells. The stem cells may be StemPro® neural stem cells available from ThermoFisher Scientific. StemPro® Neural Stem Cells are derived from human fetal brain from qualified, traceable donors. The cells are isolated, cultured, and expanded under Good Manufacturing Practice (GMP) manufacturing standards in a California-licensed facility using a proprietary Reduced Oxygen Tension manufacturing process. Manufacture of cells in a reduced oxygen tension environment results in higher yields of highly potent immature stem cells compared to cells expanded in normal oxygen culture conditions. The suspension composition may be 0.75 wt. % hyaluronic acid in divalent ion-free PBS. The hyaluronic acid has a molecular weight of 1.1 to 1.9 MDa and may be obtained from LifeCore Biomedical, LLC

With reference generally to FIG. 35, the SCARA positioning arm 910 is used to localize the injection assembly over the pig 1001. The vertical post 904 is positioned so that the guide needle 943 is approximately 4 cm above the spinal cord of pig 1001. Using the micro-adjustment controls 911 and 931 (FIG. 29B), the guide needle 942 is lowered to about 1 mm right of midline and 1 cm above the dorsal aspect of the cord. Next, the nitinol delivery catheter/injection needle 943 is advanced and the macro\micro goniometer 950 is used to align the Nitinol needle 943 with the surface of the cord and parallel to the long axis of the cord. The fluoroscope 1000 is used to confirm alignment. The guide needle rotational (rotational micromanipulator) and angular positions (goniometer) are recorded.

Upon instruction by the Neurosurgeon, the Nitinol needle 943 and guide needle 942 is retracted. The micro goniometer 950 is then used to angle the guide needle 9 degrees into the spinal cord. An Anesthesiologist then hyperoxygenates the pig and then stops ventilation upon command by the Neurosurgeon. Time off the ventilator is recorded. Using the micro-adjustment controls 911 and 931 to lower the guide needle 942 the guide needle 942 is positioned to just slightly depress the pia. A small incision/entry hole (“nick”) may facilitate entry of the Nitinol needle 943 into the cord parenchyma. The Neurosurgeon then asks that flouroscopy begins.

The Neurosurgeon calls for advancement of the Nitinol needle 943 under fluoroscopic guidance 1000. The Nitinol needle 943 is advanced to a fully extended position. A pre-flow of cells during nitinol advancement is set at 0.07 uL/mm. The fluid flow rate is then set to 0.34 uL/mm for retraction flow rate. See Table 1. Upon order of the Neurosurgeon the cell infusion and simultaneous Nitinol needle retraction is started. When the Nitinol needle 943 is fully retracted, the Neurosurgeon is informed, whereupon the Neurosurgeon raises the guide needle 942 away from the cord (at least 1-2 cm).Ventilation is then recommenced and the Neurosurgeon checks for retrograde leakage of the injection composition comprising human neural stem cells. The pia is then stitched to mark the location of the injection trail entrance. FIG. 66 shows cross-sections and longitudinal sections of STEM121 labeled human neural stem cells after 1 week delivered in a trial in a porcine spinal cord, using a similar method to the one described in Example 1.

### Example 2

### In Vitro Therapeutic Trails Injection

FIG. 50 illustrates trails injected at an angle in a “tent” formation around a prophetic injection site. Two opposing 2-cm long trails injected at 10 degree angles into a 0.6 wt. % agarose gel slab. The trails are composed of 0.75 wt. % hyaluronic acid in PBS and methylene blue was added for visualization purposes. FIG. 51A is a top view and 51b is a side view illustrating the angular injections and the described “tent” feature which may be used to inject a trail of cells and/or therapeutic substances proximal to an injury site in the spinal cord. With regard to the injection procedure, reference may be made to Example 1 above.

### Example 3

### In Vitro Injection Angle Testing

An experimental test of the accuracy of injecting trails of cells and/or a therapeutic substance was conducted in an in vitro test model to determine the accuracy and extrusion depth of injections performed with an embodiment of the present invention. A certain embodiment of injection device 900 employing a goniometer 950 was utilized through the test procedure. Thus, a preliminary test of the accuracy of the goniometer angle mechanism was performed. The test was accomplished by measuring the extrusion depth at various goniometer angles.

Materials. Tests were performed utilizing gel slabs composed of 0.6 wt. % agarose in diH₂0. The liquid composition injected was a solution of 0.75 wt. % hyaluronic acid (“HA”) with methylene blue added for color. Trails of methylene blue were measured with a ruler.

Procedure. An injection needle 943 composed of nitinol was extruded 20 mm above the test gel slab. The goniometer on an embodiment of the device substantially similar to Embodiment 8 was used to angle the nitinol injection needle parallel to the surface of the gel. An angle of 8° was recorded. The nitinol delivery catheter/injection needle was then retracted within the guide needle 942. The goniometer was adjusted to the desired angle of approach. With regard to the injection procedure reference may be made to Example 1 above for the general injection protocol. Further, the injection protocol generally followed the procedure outlined in FIG. 36 and the accompanying text in this specification.

A pre-flow of the HA/methylene blue composition was set by the controller 990 at 0.07 μL/mm. The nitinol needle was then extruded 20 mm at 0.5 mm/sec into the agarose gel slab. The liquid composition of HA and methylene blue was flowed at a flow rate of 0.35 μL/mm upon retraction of the nitinol needle by setting the controller 990 to retract the nitinol injection needle. The methylene blue trails were measured with a ruler.

Testing Conditions. The following testing conditions for 20 mm trails were noted.

TABLE 2
Goniometer Angle Settings

| | Angle (°) (with respect | Goniometer angle (~8 degrees as |
|---|---|---|
| Depth (mm) | to gel) | parallel) |
| 2 | 5.7 | 2.3 |
| 4 | 11.5 | −3.5 |
| 6 | 17.5 | −9.5 |
| 8 | 23.6 | −14.5 |

FIG. 53 depicts the angle measurements in accordance with the injection of a 20 mm trail of the liquid composition of HA and methylene blue in accordance with this Example 3.

FIG. 54 depicts the testing setup for injection device 900 used in this Example 3. Reference can be made to FIG. 54 and the accompanying text for a more detailed discussion of the injection procedure.

Results. The results obtained according to the foregoing in vitro test protocol are shown in FIGS. 55A, 55B. FIGS. 55A and 55B show methylene blue trails 1400 in agarose slab 1300 injected at a 2 mm depth and a 5.7° angle. The result shown on ruler 1301 is 2-3 mm. FIG. 56 shows the results of injections set at 4 mm, 6 mm and 8 mm, respectively. Methylene blue trails 1400 in agarose gel slabs 1300 as measured by ruler 1301 yielded the following results: (a) at a 4 mm depth setting and an injection angle of 11.5° the measured result was 4 mm; (b) at a 6 mm depth setting and an injection angle of 17.5° the measured result was 6-7 mm; and (c) at an 8 mm depth setting and an injection angle of 23.6° the measured result was 8 mm.

FIG. 57 is an image of a guide needle positioned at the surface of an agarose gel slab and an injection needle penetrating the agarose gel slab at a setting of 8 mm and an injection angle of 23.6° yielding a trail of 8 mm in accordance with Example 3.

### Example 4

### Testing of Needle Speed Range; Range of 0.1 to 5 mm/sec

The needle speed of an embodiment of the injection device for delivering trails of cells and/or a therapeutic substance was testing according to the following method.

Method. Retract the needle until approximately 2-5 mm is showing beyond the tip of the guide needle. Zero the position readout on the display. Select the speeds 0.1 mm/sec, 1.5 mm/sec and 5 mm/sec one at a time. Advance the needle at the given speed for the specified time. Measure the change in needle protrusion and compare with the theoretical value. Confirm the distance reading on the screen and record. Measuring equipment used was Calipers—Mitutoyo Digital.

### Results.

TABLE 3
Needle Speed Results

| | | | | | | Dis- |
|---|---|---|---|---|---|---|
| | Test | Expected | Start | End | | played |
| Speed | time | length | Length | Length | Distance | Length |
| (mm/s) | (s) | (mm) | (mm) | (mm) | Advanced | (mm) |
| 0.1 | 120 s | 12 mm | 3.79 | 15.07 | (mm) 11.28 | 11.98 |
| 1.5 | 20 s | 30 mm | 2.88 | 33.28 | 30.4 | 30.32 |
| 5 | 10 s | 50 mm | 2.84 | 53.76 | 50.92 | 51.59 |
| 10 | 4 s | 40 mm | 3.93 | 46.16 | 42.23 | 42.23 |

Based on the testing performed, the system display is accurate to within about 0.7 mm. A significant portion of this error is related to the measurement method. The distance advanced is different from the expected value largely due to the reaction time for starting and stopping the system at the appropriate time.

### Example 5

### Relative Fluid Delivery Range; Relative Fluid Delivery Range 0.01-8 μL/mm. Absolute Fluid Delivery Range 0.01-25 μL/sec

Method. Testing of distances traveled by the injection needle and the amount of fluid dispensed were measure according to the following method.

Method. A syringe was filled with water and a needle assembly was attached. The syringe was primed to remove air from the injection system. The needle was then advanced until approximately 50-55 mm of the needle tip was showing beyond the tip of the guide needle. The position was zeroed in the controller display. Next, the needle speed was selected and the indicated fluid rate in FIG. 57 was set. The controller was set to dispense liquid. Thereafter, the needle was retracted at the indicated speed for the time specified in FIG. 57. The dispensed water was collected in a sample tray. The needle protrusion was measured and compared to the theoretical value. The distance reading on displayed on the controller was recorded. The liquid was then weighed and the measurement was recorded in Table 4 below. Equipment utilized included Calipers-Mitutoyo digital and a Mettler-Toledo balance XS-205.

Results. The results are reported in FIG. 57 and below. Calculated values included the following:

Abs. Fluid Rate—The fluid delivery rate in ul/s. Calculated by multiplying Needle speed (mm/s) by Fluid Rate (ul/mm).

Expected Needle Travel—Calculated by multiplying Needle Speed (mm/s) by Test time (s).

Expected Total Dispense—Calculated by multiplying Fluid Rate (ul/mm) by Expected Needle Travel (mm).

Actual Syringe Travel—Calculated by subtracting the Syringe End Volume (ul) from the Syringe Start Volume (ul). Actual Needle Travel—Calculated by subtracting the Needle Start length (mm) from the Needle End length (mm).

Actual Fluid Rate by Distance—Calculated by dividing the Actual Syringe Travel (ul) by the Actual Needle Travel (mm).

The absolute distances travelled and amount of fluid varied from the expected values in the following manner. Faster needle speeds with shorter test times exhibited poorer results. This is largely due to reaction time in starting and stopping the test which has a greater effect over shorter test times.

The weighed dispensed fluid values were generally close to the fluid dispensed by distance (reading syringe graduations), but consistently lower. This can be explained by evaporation, air dissolved in solution and small amounts of water clinging to the needle after dispense. An effort was made to get the water off of the tip of the syringe, but it was difficult to confirm this

Maximum dispense rate error observed is 6.5%. Maximum needle speed error observed was 17.8% (1.6 mm out of 9 mm expected). This was observed on a 3 second test at 3 mm/sec. If reaction time accounted for 0.5 seconds of error, the distance error would have been 1.5 mm. Needle distance measurement error is estimated at about 0.5 mm. Longer tests showed distance errors of 2% maximum. The foregoing results demonstrate that expected needle travel, expected total; fluid dispensed and expected absolute fluid rate are well within expected and acceptable tolerances as demonstrated by the values actually obtained in Example 5 and reported in FIG. 57 Example 6. Nitinol extrusion accuracy in agarose gels.

A nitinol was extruded at an 8 degree angle into 0.6 wt. % agarose gels for 2 or 4 cm. Following extrusion, methylene blue in HA was flowed through the nitinol injection needle at a rate of 0.34 uL/mm while the needle was retracted at 0.5 mm/second. Three trails were made in parallel by moving the guide needle location ˜1 cm using the micro adjustment mechanism. FIGS. 63A and 63B are photographs depicting three trails of hyaluronic acid-methylene blue in agarose demonstrating consistent trail angles in FIG. 63B.

### Example 7

### Delivery of Human Neural Stem Cells Trails in Nude Rat Spinal Cord

A T10 to T11 laminectomy was performed in a nude rat. StemPro® Neural Stem Cells were combined with 0.75 wt. % HA at a concentration of 100,000 cells/uL and loaded into a 100 uL Hamilton syringe. The syringe was secured to the injection apparatus and the 29 G nitinol injection needle was primed with cells. The guide needle was lowered to the exposed surface of the rat spinal cord, the dura was cut with a 26 G needle to facilitate entry of the injection needle, and the nitinol was extruded 12 mm at an angle of 9 degrees into the rat cord. Upon full extension, flow of cell suspension was initiated (10 uL/min) along with needle retraction (0.5 mm/second). Following injection, the overlaying muscle and skin as closed and the animal was allowed to recover. FIG. 64 shows successful creation of a trail and survival of human neural stem cells in the nude rat spinal cord after one month. The cells were labeled for STEM121 and doublecortin (DCX) markers, showing cell survival and neuronal precursor differentiation.

### Example 8

A 200 kDyne contusion was induced in a nude rat at T8. Two weeks later, a T10 to T11 laminectomy was performed to allow positioning of the guide needle. StemPro® Neural Stem Cells were combined with 0.75 wt. % HA at a concentration of 100,000 cells/uL and loaded into a 100 uL Hamilton syringe. The syringe was secured to the injection apparatus and the 29 G nitinol injection needle was primed with cells. The guide needle was lowered to the exposed surface of the rat spinal cord, the dura was cut with a 26 G needle to facilitate entry of the injection needle, and the nitinol was extruded 12 mm at an angle of 9 degrees into the rat cord. Upon full extension, flow of cell suspension was initiated (10 uL/min) along with needle retraction (0.5 mm/second). Following injection, the overlaying muscle and skin as closed and the animal was allowed to recover. The rat was perfused after three months and the spinal cord was explanted for histology. FIG. 65 shows immunohistochemical staining for STEM121 and DAPI showing survival of human neural stem cells delivered in a trail through the contusion injury in a nude rat. This demonstrates that cell trails can bridge injuries in the spinal cord and survive.

### Example 9

### Subpial Delivery of Therapeutics

Subpial delivery may reduce parenchymal spinal cord damage and facilitate the delivery of therapeutics such as viral vectors. Subpial delivery is technically challenging and requires an adequate micropositioning system with appropriate angle control. Furthermore, automation of tubing entry and retraction may improve the reproducibility and ease of subpial therapeutic delivery. In order to deliver therapeutics below the pia, a blunt polyethylene catheter (PE-5 or PE-8) was assembled in the disposable trombone, as shown in FIG. 67. Subpial delivery using the automated system was tested in rats. A trombone assembly with a PE-8 catheter was attached the system's motor drive as shown in FIG. 69. Methylene blue solution (10 mg/mL in water) was loaded into a 100 uL Hamilton syringe, secured to the PE-8 catheter (fitted with a Hamilton RN fitting) and flowed through the PE-8 tubing. A laminectomy was performed in a Sprague Dawley rat to expose the dura and the dura was cut with a 27 G needle. The pia was gently lifted with a 30 G needle and the guide needle was lowered into the pial opening with an extrusion angle parallel to the cord. The PE-8 catheter was extruded using the injection system at a rate of 0.5 mm/second for 1 cm into the pial opening. FIG. 70A shows photographs of a polyethylene catheter (PE-8) extruded through the guide tubing and into the subpial space of a rat. To test the capability of injecting therapeutics subpial, the methylene blue solution was flowed through the tubing at a rate of 0.34 uL/mm while the tubing was retracted at 0.5 mm/second. FIG. 70B shows a photograph of methylene blue injected successfully through the catheter into the subpial space of the rat.

While several exemplary embodiments and features are described here, modifications, adaptations, and other implementations may be possible, without departing from the spirit and scope of the embodiments. Accordingly, unless explicitly stated otherwise, the descriptions relate to one or more embodiments and should not be construed to limit the embodiments as a whole. This is true regardless of whether or not the disclosure states that a feature is related to “a,” “the,” “one,” “one or more,” “some,” or “various” embodiments. Instead, the proper scope of the embodiments is defined by the appended claims. Further, stating that a feature may exist indicates that the feature may exist in one or more embodiments.

While several exemplary embodiments and features are described here, modifications, adaptations, and other implementations may be possible, without departing from the spirit and scope of the embodiments. Accordingly, unless explicitly stated otherwise, the descriptions relate to one or more embodiments and should not be construed to limit the embodiments as a whole. This is true regardless of whether or not the disclosure states that a feature is related to “a,” “the,” “one,” “one or more,” “some,” or “various” embodiments. Instead, the proper scope of the embodiments is defined by the appended claims. Further, stating that a feature may exist indicates that the feature may exist in one or more embodiments.

In this disclosure, the terms “include,” “comprise,” “contain,” and “have,” when used after a set or a system, mean an open inclusion and do not exclude addition of other, non-enumerated, members to the set or to the system. Further, unless stated otherwise or deducted otherwise from the context, the conjunction “or,” if used, is not exclusive, but is instead inclusive to mean and/or. Moreover, if these terms are used, a subset of a set may include one or more than one, including all, members of the set.

All references cited herein are expressly incorporated by reference in their entirety

The foregoing description of the embodiments has been presented for purposes of illustration only. It is not exhaustive and does not limit the embodiments to the precise form disclosed. Those skilled in the art will appreciate from the foregoing description that modifications and variations are possible in light of the above teachings or may be acquired from practicing the embodiments. For example, the described steps need not be performed in the same sequence discussed or with the same degree of separation. Likewise various steps may be omitted, repeated, combined, or performed in parallel, as necessary, to achieve the same or similar objectives. Similarly, the systems described need not necessarily include all parts described in the embodiments, and may also include other parts not described in the embodiments. Accordingly, the embodiments are not limited to the above-described details, but instead are defined by the appended claims in light of their full scope of equivalents.

The invention claimed is:

1. An injection system for delivering an injectable medium into an anatomical space of an animal or human subject, the system comprising:
a first linear actuator;
a syringe comprising a catheter connection at one end and a plunger attached to a plunger rod at a second end, wherein the syringe contains an injectable medium for injection into an anatomical space of an animal or human subject;
a delivery catheter having a proximal and distal end, wherein the distal end is configured to enter the anatomical space of a subject, and wherein the proximal end is attached to the catheter connection of the syringe;
a guide tube having a proximal end and a distal end, wherein the guide tube is configured to house a portion of the distal end of the delivery catheter; further wherein the proximal end of the guide tube is connected to a guide tube holder;
a stereotaxic assembly connected to the guide tube holder, thereby allowing spatial adjustments along the x, y and z-axes; wherein the stereotaxic positioning assembly is configured to move the distal end of the guide tube in spatial alignment with the external surface of the spinal cord of a subject and allows rotation about the x, y, and z axes to control the orientation of the guide tube;
wherein the delivery catheter engages the linear actuator along the length of the catheter;
wherein the distal end of the guide tube is formed in a bend relative to the proximal end of the guide tube; and
wherein the first linear actuator is configured to extend and retract the delivery catheter inside the guide tube.

2. The injection system according to claim 1, wherein the guide tube may comprise a (i) distal guide tube having a distal and proximal end, and (ii) a tubing having a distal and proximal end; wherein the distal end of the distal tube is formed in a bend relative to the proximal end of the distal guide tube; wherein the distal guide tube is joined to the guide tube holder; further wherein the proximal end of the distal guide tube is connected to the distal end of the tubing, and wherein the proximal end of the guide tube is connected to an attachment to the first linear actuator; wherein the (i) distal guide tube and the (ii) tubing house a portion of the distal end of the flexible delivery catheter.

3. The injection system according to claim 2, wherein the proximal end of the delivery catheter is connected to the catheter connection of the syringe by tubing.

4. The injection system according to claim 1, wherein the guide tube comprises a telescoping two-part trombone slide mechanism comprising: (x) an outer cylindrical cannula comprising a first lumen and (y) an inner cannula; wherein the inner cannula has a distal and proximal end, further wherein the proximal end of the inner cannula is dimensioned to slide snugly within the lumen of the outer cannula, and further wherein the distal end of the inner cannula is bent relative to the proximal end of the inner cannula.

5. The injection system according to claim 4, wherein the delivery catheter is secured to the lumen of the second cannula at a location proximal to the path of the inner cannula within the lumen of the outer cannula, and further wherein the second cannula is connected to the first linear actuator.

6. The injection system according to claim 5, wherein the system has a second linear actuator, wherein the first linear actuator is configured to extend and retract the delivery catheter through the guide tube and the second linear actuator is configured to actuate the plunger of the syringe.

7. The injection system according to claim 6, wherein the injection system further comprises a programmable controller capable of controlling (a) the first linear actuator to advance and retract the delivery catheter, and (b) to control the second linear actuator to depress the plunger rod, thereby controlling the volume and flow rate of the liquid composition from the syringe.

8. The injection system according to claim 7, wherein the delivery catheter forms a service loop at the proximal end between the first linear actuator and the syringe, thereby preventing kinking of the proximal end of the delivery catheter when the first linear actuator is actuated.

9. The injection system according to claim 8, wherein the stereotaxic assembly comprises a goniometer comprising a macro-angular adjustment and/or a micro-angular adjustment for defining the angle of entry of the delivery catheter in the x, y and z axes relative to the axis of the spinal cord of the subject positioned adjacent to the delivery catheter.

10. The injection system according to claim 9, wherein the goniometer may define the angle of entry of the delivery catheter at an angle of ±90° relative to the axis of the spinal cord of the subject.

11. The injection system according to claim 9, wherein the goniometer may define the angle of entry of the delivery catheter at an angle of ±30° relative to the axis of the spinal cord of the subject.

12. The injection system according to claim 9, wherein the goniometer may define the angle of entry of the delivery catheter at an angle of ±15° relative to the axis of the spinal cord of the subject.

13. The injection system according to claim 1, wherein the distal end of the delivery catheter is shaped in a needle point.

14. The injection system according to claim 4, wherein the injection system further comprises a vertical height adjustable post.

15. The injection system according to claim 9, wherein the injection system further comprises a vertical height adjustable post.

16. The injection system according to claim 4, wherein the injection system further comprises an adjustable articulated arm.

17. The injection system according to claim 9, wherein the injection system further comprises an adjustable articulated arm.

18. The injection system according to claim 9, wherein the micro-positioning adjustment further comprises: a first horizontal support arm; a second horizontal support arm oriented at right angles to the first horizontal support arm; and a rotatable stage member; wherein the first horizontal support arm comprises one or more adjustable vertical support rail attached to a first vertical support rail micro-adjustor for adjusting the first horizontal support arm along the z axis; further wherein the first horizontal support arm further comprises a first horizontal rail attached to a first horizontal rail micro-adjustor for adjusting the first horizontal rail in the x axis; further wherein the second horizontal support arm comprises one or more second horizontal support arm rail attached to a second horizontal support arm micro-adjustor for adjusting the second horizontal support arm in the y axis; further wherein the rotatable stage has a top surface and a bottom surface, wherein the top surface is attached to the underside of the second horizontal support arm and wherein the rotatable stage has a bottom surface; further wherein the goniometer is mounted on one or more rails attached at the top of the goniometer rail to the bottom surface of the rotatable stage.

19. The injection system according to claim 9, wherein the outer cannula is attached to a first mounting block that connects to the first linear actuator

20. The injection system according to claim 1, wherein the delivery catheter comprises a synthetic polymeric catheter.

21. The injection system according to claim 9, wherein the delivery catheter comprises a synthetic polymeric catheter.

22. The injection system according to claim 20, wherein the polymeric catheter comprises polyethylene.

23. The injection system according to claim 21, wherein the polymeric catheter comprises polyethylene.

24. The injection system according to claim 1, wherein the delivery catheter comprises an elongated tube made of a shape memory and/or superelastic alloy.

25. The injection system according to claim 4, wherein the delivery catheter comprises an elongated tube made of a shape memory and/or superelastic alloy.

26. The injection system according to claim 9, wherein the delivery catheter comprises an elongated tube made of a shape memory and/or superelastic alloy.

27. The injection system according to claim 25, wherein the elongated tube comprises nitinol.

28. The injection system according to claim 26, wherein the elongated tube comprises nitinol.

29. The injection system according to claim 27, wherein the distal end of the delivery catheter is formed into a needle shape.

30. The injection system according to claim 28, wherein the distal end of the delivery catheter is formed into a needle shape.

31. The injection system according to claim 1, wherein the angle is approximately 90 degrees.

32. The injection system according to claim 9, wherein the angle is approximately 90 degrees.

33. The injection system according to claim 1, wherein the angle is an obtuse angle.

34. The injection system according to claim 9, wherein the angle is an obtuse angle.

35. The injection system according to claim 1, wherein the angle is approximately 91 to 180 degrees.

36. The injection system according to claim 9, wherein the angle is approximately 91 to 180 degrees.

37. The injection system according to claim 1, wherein the anatomical space comprises a brain, a spinal cord, a subarachnoid space, a subpial space, a dura matter or a dural lining of the spinal cord, an intrathecal space, a pericardial space, a pleura, a seurosa, an intra-pleural space, a kidney, a renal capsule, a blood vessel or a blood vessel wall, a peritoneal cavity, an intra-abdominal space, an intrathoracic space, or any space in the body bounded by a membrane or membranous entity.

38. The injection system according to claim 1, wherein the medium comprises a pharmaceutically active substance, therapeutic cells, fluids, biological fluids, drugs, gene therapy vectors, irrigation fluids, growth factors, nuclear medicine agents, antibiotics, anti-viral agents, contrast agents, chemotherapies, or other diagnostic substances or therapeutic substances.

39. The injection system according to claim 38, wherein the therapeutic cells are selected from the group consisting of: neural stem cells, pre-differentiated cells in the neuronal lineage, glial cells, glial restricted progenitor cells, Schwann cells, olfactory ensheathing cells, fibroblasts, mesenchymal stem cells, adipose derived stem cells, induced pluripotent stem cells, embryonic stem cells, bone marrow derived stem cells, hematopoietic stem cells, genetically modified cells, and the differentiated progeny of any of the above.

40. The injection system according to claim 39, wherein the neural stem cells are undifferentiated progeny of human neural stem cells.

41. The injection system according to claim 39, wherein the neural stem cells are differentiated progeny of human neural stem cells.

42. The injection system according to claim 38, wherein pharmaceutically active substance is selected from the group consisting of Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, protein kinase inhibitors, small interfering RNAs, analogs, derivatives, and modifications thereof, and combinations thereof or other therapeutic agents.

43. The injection system according to claim 38, wherein the gene therapy vector comprising one or more viral vectors, nucleic acids, polymeric transfection agents.

44. The injection system according to claim 37, wherein the anatomical space is a brain.

45. The injection system according to claim 37, wherein the anatomical space is a spinal cord.

46. The injection system according to claim 37, wherein the anatomical space is a subarachnoid space.

47. The injection system according to claim 37, wherein the anatomical space is a subpial space.

48. The injection system according to claim 37, wherein the anatomical space is a dura.

49. The injection system according to claim 9, wherein the anatomical space comprises a brain, a spinal cord, a subarachnoid space, a subpial space, a dura matter or a dural lining of the spinal cord, an intrathecal space, a pericardial space, a pleura, a seurosa, an intra-pleural space, a kidney, a renal capsule, a blood vessel or a blood vessel wall, a peritoneal cavity, an intra-abdominal space, an intrathoracic space, or any space in the body bounded by a membrane or membranous entity.

50. The injection system according to claim 9, wherein the medium comprises a pharmaceutically active substance, therapeutic cells, fluids, biological fluids, drugs, gene therapy vectors, irrigation fluids, growth factors, nuclear medicine agents, antibiotics, anti-viral agents, contrast agents, chemotherapies, or other diagnostic or therapeutic substances.

51. The injection system according to claim 50, wherein the therapeutic cells are selected from the group consisting of: neural stem cells, pre-differentiated cells in the neuronal lineage, glial cells, glial restricted progenitor cells, Schwann cells, olfactory ensheathing cells, fibroblasts, mesenchymal stem cells, adipose derived stem cells, induced pluripotent stem cells, embryonic stem cells, bone marrow derived stem cells, hematopoietic stem cells, genetically modified cells, and the differentiated progeny of any of the above.

52. The injection system according to claim 51, wherein the neural stem cells are undifferentiated progeny of human neural stem cells.

53. The injection system according to claim 51, wherein the neural stem cells are differentiated progeny of human neural stem cells.

54. The injection system according to claim 50, wherein pharmaceutically active substance is selected from the group consisting of Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, protein kinase inhibitors, small interfering RNAs, analogs, derivatives, and modifications thereof, and combinations thereof or other therapeutic agents.

55. The injection system according to claim 50, wherein the gene therapy vector comprising one or more viral vectors, nucleic acids, polymeric transfection agents.

56. The injection system according to claim 49, wherein the anatomical space is a spinal cord.

57. The injection system according to claim 49, wherein the anatomical space is a subarachnoid space.

58. The injection system according to claim 49, wherein the anatomical space is a subpial space.

59. The injection system according to claim 49, wherein the anatomical space is a dura.

60. The injection system according to claim 1, further comprising a syringe pump for pumping the liquid medium comprising therapeutic cells and/or one or more therapeutic substance from the syringe to the flexible delivery catheter.

61. A method for delivering a trail of therapeutic cells and/or one or more therapeutic substance or diagnostic substance or other injectable medium into an anatomical space of an animal or human subject, the method comprising: introducing the distal end of the delivery catheter into the anatomical space of a subject through the distal end of the guide tube of the injection system according to claim 1; advancing the delivery catheter through actuation of the linear actuator along a trail inside the anatomical space; and retracting the delivery catheter along the trail by reversing the action of the linear actuator while delivering an injectable medium of therapeutic cells and/or one or more therapeutic substance or diagnostic substance or other injectable medium through the delivery catheter along the trail.

62. A method for delivering a trail of therapeutic cells and/or one or more therapeutic substance or diagnostic substance or other injectable medium into an anatomical space of a human or animal subject, the method comprising: introducing the distal end of the delivery catheter into the anatomical space of an animal or human subject through the distal end of the guide tube of the injection system according to claim 9; advancing the delivery catheter through actuation of the linear actuator along a trail inside the anatomical space; and retracting the delivery catheter along the trail by reversing the action of the linear actuator while delivering an injectable medium of therapeutic cells and/or one or more therapeutic substance or diagnostic substance or other injectable medium through the flexible delivery catheter along the trail.

63. The method according to claim 61, wherein therapeutic substance is selected from the group consisting of Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other therapeutic agents.

64. The method according to claim 62, wherein therapeutic substance is selected from the group consisting of Rho inhibitors, enzymes (such as arylsulfatase or Chondroitinase), growth factors (such as: insulin-like growth factor 1, epidermal growth factor, vascular endothelial growth factor, platelet derived growth factor, brain-derived neurotrophic factor, neurotrophin-3, glial cell-line derived neurotrophic factor, hepatocyte growth factor), calpain inhibitors, anti-inflammatory drugs, analgesics, anesthetics, antihistamines, antitussives, decongestants, antibiotics, antifungal medications, calcium channel blockers, beta blockers, other central nervous system acting drugs or agents (magnesium, or other salts), steroids (methyl prednisolone, dexamethasone, or other), hormones, or other therapeutic agents.

65. The method according to claim 61, wherein the delivery of the trail of therapeutic cells and/or one or more therapeutic substance or diagnostic substance or other injectable medium is imaged using magnetic resonance imaging, computed tomography, fluoroscopy, ultrasound, or other radiological modalities.

66. The method according to claim 62, wherein the delivery of the trail of therapeutic cells and/or one or more therapeutic substance or diagnostic substance or other injectable medium is imaged using magnetic resonance imaging, computed tomography, fluoroscopy, ultrasound, or other radiological modalities.

67. The method according to claim 61, wherein the anatomical space is a spinal cord.

68. The method according to claim 62, wherein the anatomical space is a spinal cord.

69. The method according to claim 61, wherein the anatomical space is a brain.

70. The method according to claim 62, wherein the anatomical space is a brain.

71. A method of treating an injury or disease of an anatomical space of an animal or human subject, comprising the step of delivery a trail of therapeutic cells and/or one or more therapeutic substance, or diagnostic substance, or other injectable medium into the anatomical space of a subject according to the method of claim 61.

72. A method of treating an injury or disease of an anatomical space of an animal or human subject, comprising the step of delivery a trail of therapeutic cells and/or one or more therapeutic substance, or diagnostic substance, or other injectable medium into the anatomical space of a subject according to the method of claim 62.

73. A method of defining the delivery of the trail of therapeutic cells and/or one or more therapeutic substances or diagnostic substances or injectable medium into an anatomical space of an animal or human subject, the method comprising: (i) obtaining a magnetic resonance image of the anatomical space; (ii) defining the angle of entry and length of the trail to be delivered; and (iii) applying the angle of entry and length of the trail to be delivered to the surgical approach by aligning the angles with intraoperative fluoroscopy or computed tomography markers.
